(19) United States
(12) Patent Application Publication
(10) Pub. No.: US 2017/0231058 A1
(43) Pub. Date: Aug. 10, 2017

(54) Lighting Systems

(71) Applicant: INNOSYS, INC., Salt Lake City, UT (US)

(72) Inventor: Laurence P. Sadwick, Salt Lake City, UT (US)

(21) Appl. No.: 15/502,172

(22) PCT Filed: Aug. 4, 2015
(86) PCT No.: PCT/US15/43691

§ 371 Date: Feb. 6, 2017

Related U.S. Application Data
(60) Provisional application No. 62/033,043, filed on Aug. 4, 2014.
(60) Provisional application No. 62/033,041, filed on Aug. 4, 2014.

(51) Int. Cl. H05B 33/08; A61M 21/02; A61N 5/06; H05B 37/02; F21K 9/278

(52) U.S. Cl. CPC H05B 33/0896; H05B 33/089; H05B 37/0236; F21K 9/278; H05B 33/0872; H05B 37/0281; H05B 33/0845; H05B 37/0272; A61N 5/0618; A61M 21/02; F21Y 2115/15

(57) ABSTRACT

A solid state lighting system includes a number of light sources with multiple light colors that can be used to replace fluorescent lamps.

### BACKGROUND

A major source of wasted and excessive energy usage is inefficient lighting such as incandescent bulbs and older types of ballasts used for T8 and T12 fluorescent lights. Interest has grown rapidly in replacing incandescent lights with more efficient lighting, such as fluorescent lighting and light emitting diodes (LEDs). However, great room for improvement remains in efficient lighting.

### SUMMARY

Various embodiments of the present invention provide solid state lighting systems, solid state lighting system power supplies, solid state lighting controllers and/or dimmers, etc.

The embodiments shown and discussed are intended to be examples of the present invention and in no way or form should these examples be viewed as being limiting of and for the present invention.

This summary provides only a general outline of some embodiments of the invention. The phrases “in one embodiment,” “according to one embodiment,” “in various embodiments”, “in one or more embodiments”, “in particular embodiments” and the like generally mean the particular feature, structure, or characteristic following the phrase is included in at least one embodiment of the present invention, and may be included in more than one embodiment of the present invention. Importantly, such phrases do not necessarily refer to the same embodiment. Additional embodiments are disclosed in the following detailed description, the appended claims and the accompanying drawings.

### BRIEF DESCRIPTION OF THE FIGURES

A further understanding of the various embodiments of the present invention may be realized by reference to the Figures which are described in remaining portions of the specification. In the Figures, like reference numerals may be used throughout several drawings to refer to similar components.

FIG. 1 depicts an array of solid state lighting panels in accordance with some embodiments of the invention;

FIG. 2 depicts an array combining solid state lighting panels and solid state point light sources in accordance with some embodiments of the invention;

FIG. 3 depicts another array combining solid state lighting panels and solid state point light sources in accordance with some embodiments of the invention;

FIG. 4 depicts groups of arrays of variously colored point light sources in accordance with some embodiments of the invention;

FIG. 5 depicts another array of lights sources in accordance with some embodiments of the invention;

FIG. 6 depicts a linear array of variously colored point light sources in accordance with some embodiments of the invention;

FIG. 7 depicts a block diagram of a lighting system with solid state light replacement of fluorescent lamps in accordance with some embodiments of the invention;

FIG. 8 depicts a block diagram of a lighting system with solid state light replacement of fluorescent lamps with multiple ballasts in accordance with some embodiments of the invention;

FIG. 9 depicts a block diagram of a lighting system with multiple solid state light fluorescent lamp replacements with internal drivers in accordance with some embodiments of the invention;

FIG. 10 depicts a solid state fluorescent lamp replacement in accordance with some embodiments of the invention;

FIG. 11 depicts a solid state fluorescent lamp replacement with external driver/converter/power supply etc. in accordance with some embodiments of the invention;

FIG. 12 depicts a solid state fluorescent lamp replacement positioned to be inserted in a fluorescent fixture in accordance with some embodiments of the invention;

FIG. 13 depicts a solid state fluorescent lamp replacement mounted in a fluorescent fixture in accordance with some embodiments of the invention;

FIGS. 14-22 depict arrays of solid state lights in various configurations in accordance with some embodiments of the invention;

FIGS. 23-26 depict solid state lighting panels mounted in fluorescent lamp fixtures in various configurations in accordance with some embodiments of the invention;

FIGS. 27-28 depict lighting systems with fans in accordance with some embodiments of the invention;

FIGS. 29-31 depict block diagrams of a solid state fluorescent lamp replacement with ballasts in accordance with some embodiments of the invention;

FIGS. 32-38 depict example power supplies for solid state fluorescent lamp replacement lighting systems in accordance with some embodiments of the invention;

FIGS. 39-40 depict a solid state fluorescent lamp replacement with a light emitting panel and clips for attaching to a lamp replacement bar mountable in a fluorescent fixture in accordance with some embodiments of the invention;

FIGS. 41-46 depict a solid state light emitting panel 4100 with bi-pin connector mounts to tombstones in a fluorescent lamp fixture to replace two fluorescent lamps in accordance with some embodiments of the invention;

FIGS. 47-55 depict a solid state light emitting panel 4100 with bi-pin connector mounts to tombstones in a fluorescent lamp fixture to replace one fluorescent lamp in accordance with some embodiments of the invention;

FIG. 56 depicts a fluorescent replacement power buss in accordance with some embodiments of the invention;

FIG. 57 depicts a fluorescent replacement power buss with connected solid state spot lights in accordance with some embodiments of the invention;

FIG. 58 depicts a fluorescent fixture with a fluorescent replacement power buss and solid state spot lights in accordance with some embodiments of the invention;

FIG. 59 depicts a pair of fluorescent replacement power busses linked by a cross-bar in accordance with some embodiments of the invention;

FIGS. 60-62 depicts a cross-bar for linking a pair of fluorescent replacement power busses in accordance with some embodiments of the invention;

FIGS. 63-64 depict an edge lit light bar/panel in accordance with some embodiments of the invention;

FIGS. 65-67 are block diagrams of embodiments of a solid state fluorescent replacement lighting system is depicted in accordance with some embodiments of the invention; and

FIG. 68 depicts a block diagram of a home automation system incorporating a lighting system in accordance with some embodiments of the invention.

### DETAILED DESCRIPTION OF THE INVENTION

Various embodiments of the present invention provide solid state lighting systems that can be used to replace fluorescent lamps or in new installations or other applications, and can include solid state lighting panels, solid state point light sources such as, but not limited to, light emitting diodes (LEDs), organic light emitting diodes (OLEDs), quantum dots (QDs), etc., and/or combinations of solid state lighting panels and solid state point light sources. In some embodiments, the solid state lighting systems can be implemented in various types and designs of lighting fixtures, such as, but not limited to, troffers, task lamps, bed lamps, table lamps, under counter, over counter, vanity, wall, ceiling, sconce, luminaires, etc., with or without additional devices such as fans, sensors, cameras, etc.

Some embodiments of the present invention can be implemented as a fluorescent tube replacement of any length and any diameter that contains multiple color light sources with or without a white light source or with more than one white light source including white light sources having different color temperatures (CCTs), which can be controlled (i.e., turned on, dimmed, pulsed, flashed, etc.), for example, but not limited to, in ways to produce shorter visible wavelength which could, for example, but not limited to, be dominant or entire/totally containing light in some implementations for waking up and waking hours and produce longer visible wavelength containing light with the absence of or greatly reduced shorter wavelength content light for sleeping and resting as well as other types of lights including but not limited to A lamps (including E26 and E27 socket lamps), PAR lamps (including PAR30 and PAR38), R lamps (including R30, R40), flood lamps, PL 2 or 4 pin lamps, MR lamps (including MR16), GU lamps (including GU10), T12, T10, T9, T8, T5, T4, etc., 1 ft, 2 ft, 3 ft, 4 ft, 5 ft, 6 ft, 8 ft, less than 1 ft, greater than 4 ft, greater than 8 ft, 18 inches, etc., high intensity discharge lamps (HID) of any type and form, 2×2 ft, 4×4 ft, 2×4 ft, 4×2 ft, 1×1 ft, etc. low voltage lamps, low voltage magnetic lighting, track lighting, etc., virtually any type of light form factor light source, combinations of these, etc.

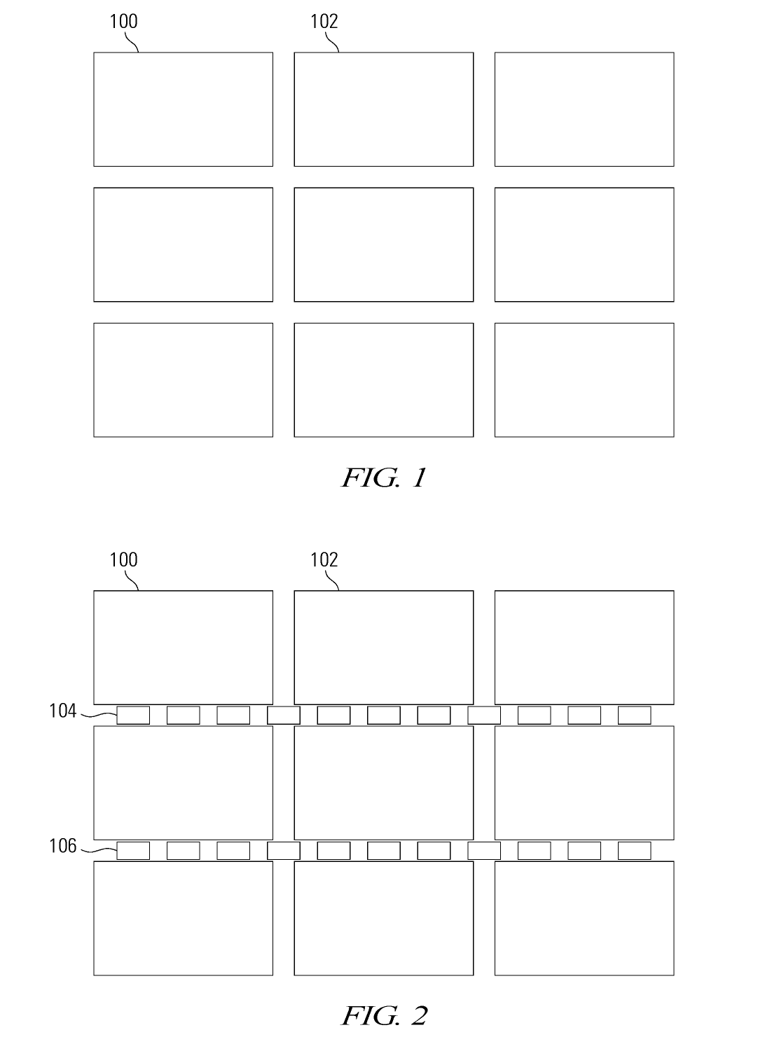

Turning to FIG. 1, in some embodiments arrays of solid state lighting panels (e.g., 100, 102) can be controlled independently and/or collectively to provide desired colors and illumination levels, either in static configurations or dynamically changing in response to control signals, programmed patterns, external stimuli, time, location, information gathered locally and/or remotely, etc. The patterns shown in FIG. 1 and other Figures are intended to represent implementations and embodiments of the present invention in terms of lighting and can be of any color or colors including white light and in particular may be of more than one color including more than one white color temperature and/or more than one color with each element of the block representing either a single color or multiple colors of light. The lighting may be controlled, dimmed, selected, monitored by wireless (including but not limited to Bluetooth, WiFi, ISM, IEEE 801, 2.4 GHz, etc.) or wired (DMX, DALI, RS 232, RS 485, serial, SPI, U2C, USB, etc.) means.

All of the lighting for the present invention may be dimmed either locally or remotely with the dimming based on Triac, forward or reverse dimming, wired or wireless, powerline, etc. dimming, control, monitoring, logging, etc. including the wired and wireless methods, technologies, approaches, interfaces, protocols, etc. discussed in the previous paragraph and throughout herein.

The present invention can be synchronized, set, programmed, sequenced, etc. to work with internal and external stimuli, signals and input to provide lighting to improve health care, function, operation, well-being and also provide other features including warnings, alerts, alarms, etc. that can be, for example, but not limited to, audible, vision, audio, visual, lighting, sound, light, e-mail, text messages, phone calls, web content, web alerts, e-mail alerts, text alerts, other electrical, communication, mechanical, sound, visual, audio, invisible, beyond the audio frequency range, etc.

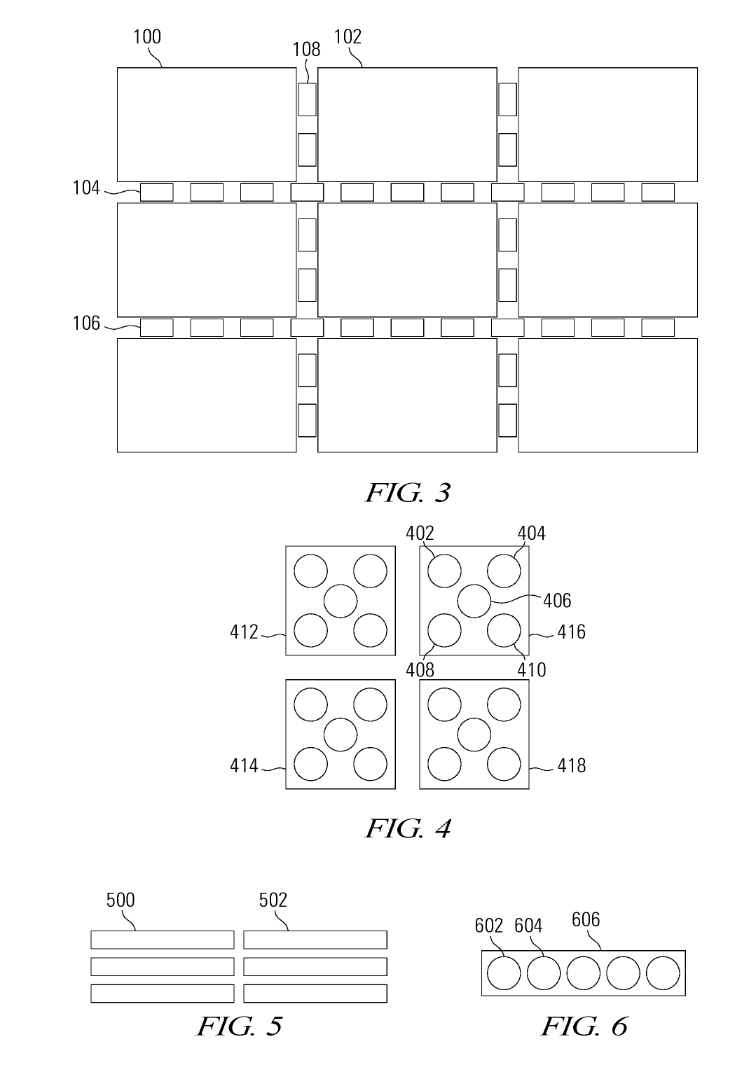

Turning to FIG. 2, one or more solid state point light sources (e.g., 104, 106) or smaller light sources such as, but not limited to, LEDs and QDs can be included with panel or larger light sources (e.g., 100, 102) to provide highly controllable, configurable and adaptable combinations of ambient lighting, mood lighting, task lighting, lighting of variable colors/wavelengths and illumination levels. As shown in FIG. 3, point light sources (e.g., 104, 106, 108) can be included with panel light sources (e.g., 100, 102) in any configuration, such as, but not limited to, between or around light panels.

The term “point light source” is used herein to refer to one or more solid state light sources, such as, but not limited to, LEDs and QDs, or even to OLEDs that are smaller relative to larger lighting panels used in a system. Turning to FIG. 4, point light sources 402, 404, 406, 408, 410 of different colors or characteristics (e.g., red, green, blue, amber, white, etc.) can be combined or grouped to form a group point light source (e.g., 416) of controllable overall color), and grouped point light sources (e.g., 412, 414, 416, 418) can be combined in groups or arrays to provide the desired lighting capabilities, with or without panel light sources in a lighting system.

Turning to FIG. 3, panel light sources (e.g., 500, 502) of any shape or size can also be combined in groups or arrays to provide desired lighting capabilities, with or without point light sources in a lighting system. As shown in FIG. 4, point (e.g., 602, 604) or panel light sources of any color (e.g., red, green, blue, amber, white) can be combined in any topology or layout to form grouped light sources 606 of any desired shape, such as linear shapes that can be positioned between larger panel light sources. Other arrangements, colors, ordering of point and panel light sources can be used in various embodiments of the present invention to provide lighting systems for any application or environment.

Again, lighting systems in various embodiments of the invention can be provided and combined as desired in any form factor or application, such as, but not limited to, smart T4, T5, T8, T95, T10, T12, CFL, PL, PL-C, other linear, U-shape, other shapes, etc. fluorescent lamps types, etc., E26, E27, A-lamp, MR-16, GU-10, PAR 30, PAR 38, R 30, 2×2, 2×4, 2 ft.×2 ft., 2 ft.×4 ft., 1 ft.×3 ft., 3 ft.×1 ft., ½ ft.×2 ft., ½ ft. by 4 ft., other lighting types, forms, models, etc. panels, tiles, etc., smaller, larger custom, other sizes, sizes to fit into existing luminaires and fixtures, etc., down light, can light, under cabinet, over cabinet, sconce, troffer, pendant fixtures, chandelier fixtures, under cabinet, over cabinet, track lighting, etc., lighting panels, tiles, bars, etc. The light sources used or powered in the invention can include waveguided, edge emitting, edge lit, back lit, direct lit, directly lit, surface lit, surface emitter, and edge emitter, combinations of these, etc. LED lighting and lighting panels, tiles, etc. and combinations of these. The lighting panels, tiles, etc. can be white, multiple white colors, RGB, RGBW, RGBA, RGBAW, RGBAWW, RGBAWWW, etc., combinations of these, etc.

In some embodiments of the present invention, if the power is too high for the heat sink(s), the embodiments of the present invention can limit the power supplied to the LEDs then cut back the power to certain LEDs as specified, desired, automatically selected, combinations of these, etc. To determine/set/evaluate limit, implementations of the present invention can calculate or use one or more temperature sensor(s), thermistors thermocouples (TCs), positive coefficient thermistors, negative coefficient thermistors, IC temperature measurement, semiconductor temperature measurement, etc.

The present invention works with all types of ballasts including instant start, rapid start, programmed start, dimmable ballasts, etc. Embodiments of the present invention can have internal or external power supplies/drivers, including power supplies and drivers designed to replace the ballast and provide constant current output or in some embodiments constant voltage or constant power or selectable/settable between the various types of constant output.

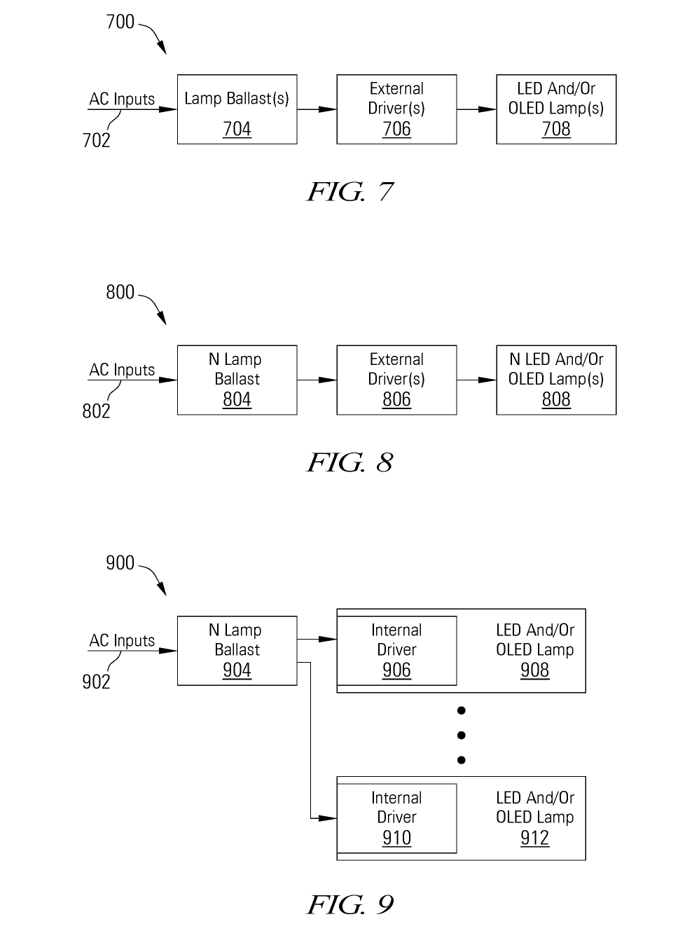

For example, as shown in FIG. 7, LED and/or OLED lamps 708 or other types or combinations of light sources in a lighting system 700 can be controlled and/or powered by external driver(s) 706, drawing power through lamp ballast(s) 704, if any, from AC inputs 702 or any other power source. As shown in FIG. 8, a lighting system 800 can include multiple or N lamp ballast(s) 804 drawing power from AC inputs 802 or any other power source, with external driver(s) 806 powering N lamps 808. As shown in FIG. 9, in some embodiments, a lighting system 900 can include multiple or N lamp ballast(s) 904 drawing power from AC inputs 902 or any other power source, with lights 908, 912 in the system 900 including LED and/or OLED or other types of light sources 908, 912 being powered by internal drivers 906, 910.

Should the ballast at some future time fail to work properly, fail to operate, stop working, etc., the present invention allows the ballast to be disconnected, removed, etc. and, for example, a new ballast or a new power supply, power source, to be used with the present invention such that the new power source could be connected to the input of the external driver or to directly to the LED and/or OLED lights, lamps, lighting, etc. Embodiments and implementations of the external driver can have the capability to run off/be powered by AC line voltage in addition to being powered by a ballast. Embodiments and implementations of the present invention can automatically select between ballast and AC line voltage or manually, including a switch, or remote control to select whether to receive power from an AC line or a ballast (including an emergency power ballast), by the type of connections, including the connectors, combinations of these, etc. In some embodiments, power status can be remotely accessible, for example indicating when a ballast is not providing suitable voltage and/or current, or when the voltage and/or current level from a ballast has changed, allowing an operator to either remove or change the ballast, or to remotely select AC line input rather than ballast input. Many of these embodiments can also be powered directly from DC power including relatively low DC voltages on the order of less than 10 V DC and, for certain implementations, as low as 3 V DC and in some implementations up to 500 VDC or higher.

In other embodiments of the present invention an input socket can be used to power the LED and/or OLED lights, lamps, lighting, etc. In other embodiments of the present invention an input and output socket can be used to power the LED and/or OLED lights, lamps, lighting, etc. such that unless power/current is applied to the input, the LED and/or OLED or QD lights will not turn on. Such sensing of input power levels and control of output power to load(s) can be performed locally and automatically by sensing and control circuits in the lighting system, or by reporting conditions to allow remote control of the system, or a combination of these or other control systems.

The present invention can use a ballast as a power supply including but not limited to fluorescent lamp ballasts, high intensity discharge (HID) lamp ballasts, sodium lamp ballasts, other gaseous and/or filament lamps, etc. of any type, form, power level, power output, power rating, lumen rating, etc., in which the power from the output of the ballast(s) can be used as a power source such as an AC or DC power source including where the power from multiple outputs of a single ballast or plurality of ballasts are combined. Embodiments of the present invention can use power combining with or without isolation of any type or form including but not limited to capacitors, transformers, inductors, diodes, resistors, transistors including but not limited to other components and devices and active devices including switches, transistors, triacs, thyristors, silicon controlled rectifiers (SCRs), synchronized transistors, integrated circuits (ICs), application specific integrated circuits (ASICs) of any type, any material, any material compositions including but not limited to heterojunctions, heteromaterials, etc. to provide and perform power combining of one or more ballast outputs. The power combined outputs can be single stage, two stage, multiple stage, etc. including, but not limited to, push-pull, forward converters, flyback, buck, buck-boost, boost-buck, boost, Cuk, SEPIC, half-bridge, full-bridge, voltage mode, current mode, current fed, voltage fed, etc.

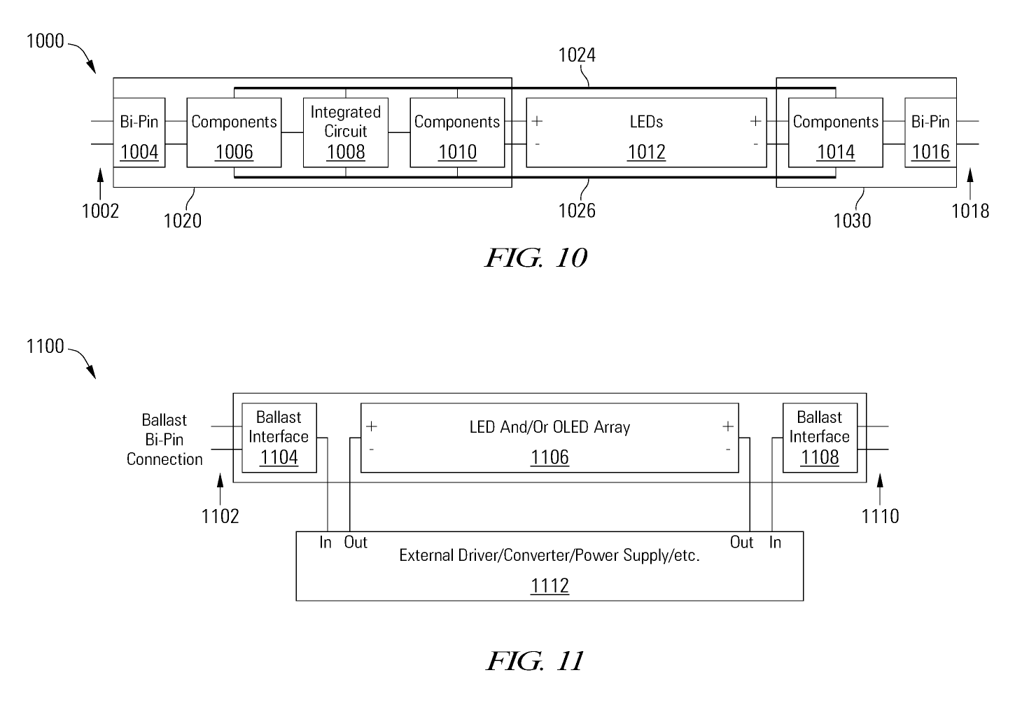

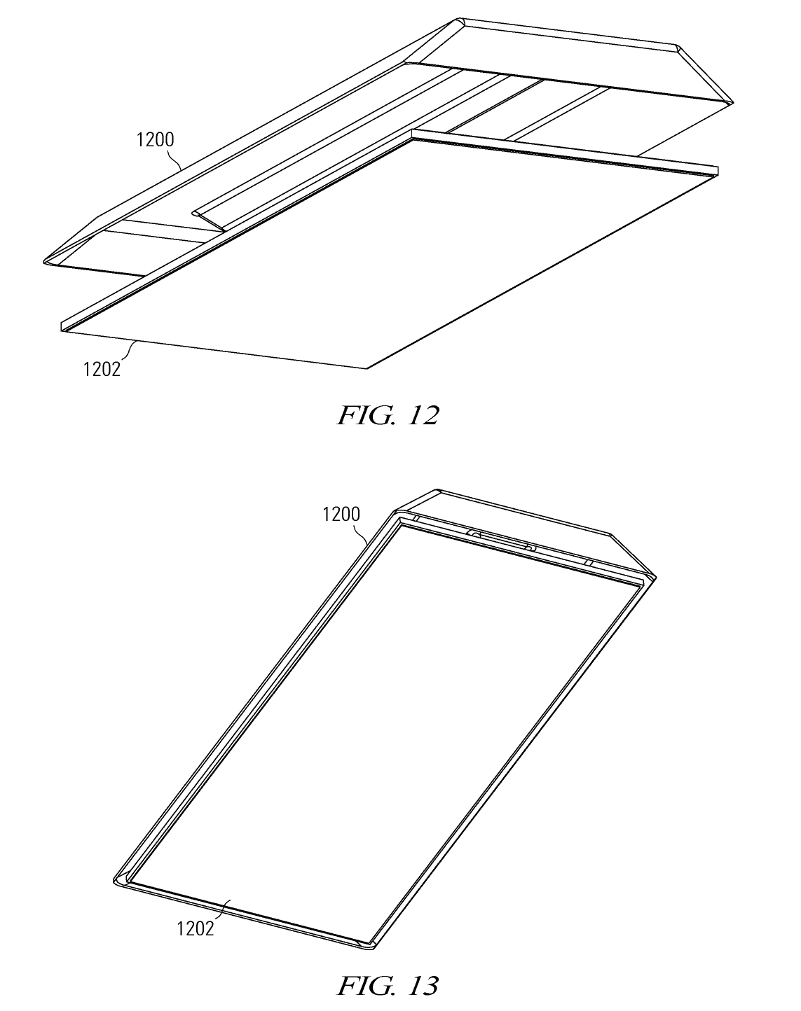

In some embodiments of the present invention, the current/power of one or more lamp outputs may be combined in any number of ways including multiple ways of providing power to individual direct fluorescent lamp replacements including the example embodiments of the present invention shown in FIGS. 10-11, power combiners, power combining, etc.

Turning to FIG. 10, a solid state fluorescent lamp replacement 1000 is depicted in block diagram form in accordance with some embodiments of the invention. The block diagram is not drawn to scale. End caps 1020, 1030 contain bi-pin connectors 1004, 1016 and components to draw power from pins 1002, 1018 which extend into tombstone connectors in the fluorescent light fixture both to provide an electrical connection between the solid state fluorescent lamp replacement 1000 and to physically support the solid state fluorescent lamp replacement 1000. One or more printed circuit boards in the solid state fluorescent lamp replacement 1000 supports and provides electrical connections 1024, 1026 between any included active or passive components 1006, 1010, 1014, one or more integrated circuits (ICs) 1008, and one or more light sources such as, but not limited to, LEDs 1012, OLEDs, QDs, combinations of these, etc. Lights in the solid state fluorescent replacement can include light sources of any type and color, such as, for example, full spectrum, white/red/green/blue (WRGB), RGB, red/green/blue/amber (RGBA), WRGBA, WWRGB, WWRGBA, WWA, WWW, WWWA, WWWRGB, WWWRGBA, etc., and can include one or more white colors (CCTs) with or without other colors, etc.

The present invention is instant start, programmed start, and/or rapid start, dimmable ballast electronic and magnetic ballast compatible as well as, in some implementations, AC line compatible including, but not limited to, less than 80 VAC to greater than 480 VAC and DC compatible from less than 50 VDC (and down to around 3 VDC) to greater than 600 VDC or higher. The IC 1008 can be or can include, contain, be part of, etc., a microcontroller, a microprocessor, a field programmable gate array (FPGA), an ASIC, multiple chips including being assembled and packaged together or separately that perform these functions that may also include one or more wireless and/or wired interfaces to communicate and control, monitor, dim, etc. the present devices. In some embodiments of the present invention, for example, the fluorescent lamps comprise one or more, for example, panel or tile lights that can fit into, be interfaced with, be connected to, be retrofitted, etc. using the existing ballast, connections, fixtures, etc.

The present invention can be used with different fixtures and can allow additional features not currently possible including having colors such as RGB, RGBA, other color combinations, one or more colors, white plus colors, full spectrum, form factor change other than T5, T8, T12, other fluorescent lamp shapes, U shapes, curved shapes, multiple shape, etc. including changing to, for example but not limited to, approximately 2 ft.×2 ft., 3 ft.×2 ft., 3 ft.×3 ft., 2 ft.×4 ft., 3 ft.×4 ft., etc. In some embodiments, for example, light sources and/or panels of various colors can be combined, such as, but not limited to, a panel with orange, amber, etc., another panel with blue, green, etc. 480 nm or in the range of 455 nm to 490 to 500 nm wavelengths or at specific wavelengths in these ranges, etc., another panel with full spectrum RGB, RGBA, WRGBA, WWRGB, WWRGBA, WWA, WWW, WWWA, WWWRGB, WWWRGBA, RGBW, etc., and another panel with other colors including but not limited to one or more white colors and/or other colors.

Embodiments of the present invention can provide emulation/simulation/etc. of the sun's (e.g., solar) spectrum of light including from pre-dawn to post-dusk and can optionally augment/supplement/synchronize/set the spectrum especially the full visible spectrum. Embodiments of the present invention can, among other things, track the Sun or time/phase shift the Sun's spectrum and provide such spectrum lighting at any time including offset times for, for example, shift workers. Embodiments of the present invention can provide exact replicas of the Sun's spectrum or add or subtract from the Sun's spectrum. Embodiments of the present invention can provide exact timing to match the local time or other time zones so as to train and entrain, sync, etc. a person's or persons' circadian rhythm to a local or other time zone for work, travel, vacation, etc. uses and purposes. The present invention can take and gather information from numerous sources including but not limited to the web/internet, the local and global environment, and the user, etc. time, day, date, weather, etc., combinations of these, other information, calculations, formulations, equations, data, results, etc., from any source or sources of any type and form, etc. and, for example, precisely or approximately put out a spectrum that emulates that of the Sun or deviates from that of the Sun at certain times and moments and locations which can be programmed by the user or by others to, for example, account for cloudy days, rainy days, and other weather and Sun/solar related, etc. conditions, matters, issues, considerations, etc. The present invention can adjust, adapt, modify, etc., and be programmed to respond to various human factors and environmental conditions. The present invention can also be used to provide a smart, intelligent and interactive light source to treat seasonal affective disorder (SAD) among other light/phototherapy treatments/applications/needs/etc. including receiving signals from one or more sensors and detectors including, but not limited to wired and wireless signals, feedback, information, etc. from one or more devices including with wearable devices and other sensors that can detect, for example, but not limited to, heart rate, blood pressure, phase of the circadian rhythm cycle, other information about circadian rhythm, ambient light, pressure, movement, electroencephalogram/electroencephalography (EEG), electrocardiography/electrocardiogram (EKG or ECG), brain waves, oxygen level, brain waves, muscle movement, body temperature, pulse rate, actimetry, sleep actigraphs, temperature, polysomnography (PSG), mood, emotional state, etc. Wearable devices can include, but are not limited to, wrist devices, or watch-shaped devices worn on the wrist of the non-dominant arm, detectors and sensors, sleep management and monitoring sensors, systems, etc. including for awake, REM, deep sleep, various other states of sleep and wake, etc., delayed sleep phase disorder, perspiration, orientation, location, vertical or horizontal sensing, etc., speech, speech patterns, voice, weather, etc., combinations of these, etc. Such signals, input, feedback, information, etc. can be used to, for example, to set the level, spectrum and intensity, emulated sunlight spectrum, white temperature, color temperature, duration and intensity of treatment, etc. In addition, sensors can include light sensors, photosensors, spectrum analyzers including optical spectrum analyzers, light sensors with or without one or more notch filters, motion sensors, proximity sensors, radio frequency identification (RFID), sonar, radar, ultrasonic, ultrasound, voice, noise, microphones, vibrations, mechanical, acoustic, cell phones, smart phones, tablets, etc. Smart phones, tablets, laptops, computers, dedicated control and/or interface units, etc. may be used to, for example, but not limited to, transmit and/or process the information via applications or apps or can use apps to display, store, log, analyze, etc. data, results, performance, control, provide feedback, etc. The present invention can incorporate and use open platforms including but not limited to Google Fit, Apple HealthKit, Apple Watch, wearable devices, technologies, watches, monitor straps or bands, etc., and can interface not only with biometric monitor devices but also with health tracking/reporting applications, websites, medical providers, etc.

Embodiments of the present invention allows for scheduling/programming of events remotely including for persons who are unable to do so themselves which can also include remote scheduling, programming, monitoring, control, etc. The present invention can also be used to treat and/or assist in the treatment of dementia and related conditions and other diseases, illnesses, chronic health conditions, impairments, disorders including sleep disorders, etc. The present invention can also provide power for other uses, functions including but not limited to fans, motors, heaters, blowers, fan blades, security cameras, surveillance cameras, monitors, monitoring systems, web-based cameras, motorized cameras, etc., universal serial bus (USB) and other charging, auxiliary power, etc., battery backup, emergency batteries, microphones, speakers, earphones, headphones, etc., sensors, WiFi, wireless power, combinations of these, etc. In some embodiments of the present invention, the USB or other communications protocols can also be used for one or two way communications including, but not limited to communicating sensitive information such as but not limited to passwords, updates, program updates, etc. In some embodiments of the present invention, various wireless approaches can be used that for example, but are not limited to, involve WiFi and Bluetooth of any type or form to communicate with devices including but not limited to smart phones, iPod, iPad, tablets, computers, laptops, etc. along with direct communication including, but not limited to, wireless remote controls, voice control, voice recognition, etc. via Bluetooth, ISM, Zwave, Zigbee, Wink, other wireless frequencies, interfaces, protocols, approaches, methods, etc. For example, a microphone that can communicate via Bluetooth and/or ISM or other wireless frequencies can be used to communicate with the present invention to provide (for example but not limited to) commands including on/off, dimming, color changes including but not limited to white color changes, other color changes including but not limited to red, blue, green, amber, etc. In some embodiments of the present invention, a buck, buck-boost, boost-buck, and/or boost switching topology is used to provide power for the present invention. As an example, a buck circuit can be used to provide AC to DC (as well as DC to DC) regulated power to the present invention. An example of an efficient way of providing such power is to for example have the buck circuit be controlled based on the lowest and strictest required regulation voltage that typically is used for the control circuits such as, for example, the integrated circuits which could, for example, consist of but is not limited to a microcontroller, microprocessor, FPGA, DSP, CLD, etc., one or more of these or each of these, wireless or wired ICs, interfaces, devices, protocols, etc. including but not limited to, WiFi, Bluetooth, ZigBee, Zwave, PLC, IEEE 801, IEEE 802, ISM frequencies, other bands and frequencies, I2C, RS232, RS485, DMX, DALI, SPI, USB, serial, etc., combinations of these including one or more of the same or different ones, etc. that is used with one or more windings (as discussed in U.S. patent application Ser. No. 13/674,072, filed Jun. 2, 2013 for a “Dimmable LED Driver with Multiple Power Sources” which is incorporated herein by reference for all purposes) on the buck inductor to provide multiple outputs including, for example, but not limited to, typically 3 V to 5 V for the control electronics, 5 V to 15 V to 20 V for the power devices including the gate drive for the power transistors including FETs and in some embodiments bipolar junction transistors (BJTs) and Darlingtons and IGBTs. In addition to these windings, a winding or windings for, for example, can also be used to provide power to the LEDs and/or OLEDs as well as power for other needs and applications including fans, motors, USB, battery chargers, etc. Linear regulation, linear regulators, switching regulators, voltage regulators, current regulation, current regulators, shunt, regulation, shunt regulators, combinations of these, etc. may be used.

Ballasts can be used as power sources and supplies with multiple uses, applications, voltages, power, current and voltage control, etc.

The present invention can be used for providing power, controlling, dimming, turning on/off, and monitoring, logging, decision making, etc. related to providing power. Power can be derived from any source, such as, but not limited to, wall power including in a single or dual wall plug or higher count in a single gang, two gang, multiple gang box size or as a plug-in extender, etc., DC power from batteries, solar cells, wind generators, solar power in general, geo power, thermal power, fuel cells, other off grid power sources, power converters, power inverters, other types of power including but not limited to those discussed herein. The present invention can be wired, wireless, PLC, etc., combinations of these, etc. The present invention can be mounted/installed in, for example but not limited to, in a standard wall outlet box, a wall dimmer, an on/off switch, a light socket, including but not limited to an A-lamp socket, an E26 socket, etc. The present invention can monitor, store, log, etc., electrical parameters including, but not limited to, current, voltage, power, power factor, apparent power, real power, AC current, DC current, AC voltage, DC voltage, etc. The present invention can select between dimming, dimming with on/off and on/off only by automatic, manual including switch(es), remote control, detection and analysis, etc. and store, select, retrieve, recreate, etc. such settings and when to invoke, activate such settings including in some implementations depending on which person or persons or animals or groups of people or otherwise, etc. are present. The present invention can, for example, measure the AC input voltage and produce a scaled version of the AC input voltage, measure the AC input current and produce a scaled version of the AC input current, measure any DC offsets to the input current, voltage, power, etc. measure the output current, voltage, power, etc.

An example embodiment for measuring the AC input voltage involves the use of high resistance resistors and one or more op amps to measure the voltage. Such embodiments can involve level shifting if needed. Measuring either the input current or voltage or both can be accomplished by the use of op amps; for example, the current can be measured by measuring the voltage across a relatively low value resistance and then applied, and voltage shifted if needed, using an op amp or op amps and other components as needed. In some embodiments of the present invention, various wireless approaches can be used that for example, but not limited to, involve WiFi and Bluetooth for devices including but not limited to smart phones, iPods, iPads, tablets, computers, laptops, etc. along with direct communication including, but not limited to, wireless remote controls, voice control, voice recognition, etc. via Bluetooth, ISM, WiFi, other wireless frequencies, etc. For example, a microphone that can communicate via Bluetooth and/or ISM or other wireless frequencies can be used to communicate with the present invention for example, including but not limited to, voice recognition, voice commands, audio commands, audible responses, voice directions, voice suggestions, voice recommendations, etc., other methods, approaches, techniques, technologies, etc. discussed herein, combinations of these, etc.

The present invention can be used to provide assisted care or monitoring in general including using voice commands, voice recognition, wearable device(s) information, wired and wireless panic buttons, proximity sensors, motion sensors, sound sensors, voice recognition, voice commands, etc. The present invention can take, use, analyze, make decisions, etc. based on data, signals, information, etc., from one or more sensors and detectors including, but not limited to wired and wireless signals, feedback, information, etc. from one or more devices including with wearable devices and other sensors that can detect, for example, but not limited to, heart rate, blood pressure, phase of the circadian rhythm cycle, EEG, EKG, oxygen level, brain waves, muscle movement, body temperature, pulse rate, mood, emotional state, location, GPS, elevation, sound, mechanical, movement, time duration, vibration, sound, pressure, accelerometer(s), sound spectrum, ultrasound, sonar, light spectrum, light environment, light quality, etc. Such signals, input, feedback, information, etc. can be used to, for example, to set the level, spectrum and intensity, emulated sunlight spectrum, white temperature, blue lighting, green lighting, amber lighting, orange lighting, lighting sensors, duration and intensity of treatment, etc. In addition, infrared detectors and sensors, motion sensors, proximity sensors, RFID, RFID sensors, cell phones, smart phones, tablets, etc. In some embodiments of the present invention, such sensors and/or cell phones, smart phones, tablets, PDAs, apps, firmware, software, etc., can be used to detect, determine, decides, etc. whether human(s), animal(s), etc, are present or absent, etc. In some other embodiments the health status, safe status of humans and/or animals can also be detected, for example but not limited to by detecting movement, lack of movement, sound, temperature using IR scanners or other means, etc. Smart phones, tablets, laptops, computers, dedicated control and/or interface units, etc. may be used to, for example, but not limited to, transmit and/or process the information via apps or can use apps to display, store, log, analyze, etc. data, results, performance, control, provide feedback, etc. The present invention can incorporate and use open platforms including but not limited to Google Fit, Apple HealthKit, FitBit, Apple Watch etc. Telephone-based, web-based, cloud-based, etc., cell phone based, combinations of these, etc. can be used to transmit, receive, communicate, recognize, alert, warn, contact, control, monitor, etc. In some embodiments of the present invention, various wireless approaches can be used that for example, but are not limited to, involve WiFi and Bluetooth such that devices including but not limited to smart phones, iPods, iPads, tablets, computers, laptops, etc. along with direct communication including, but not limited to, wireless remote controls, voice control, voice recognition, voice or other audio commands, commands in general, etc. via Bluetooth, ISM, other wireless frequencies, etc. For example, one or more a microphones that can communicate via wires, wired interfaces, with, for example but not limited to, preamplifiers, amplifiers, analog to digital converters (ADCs), digital to analog converters (DACs), etc., other methods, approaches, etc.; the amplifiers and other parts, components, subsystems, systems, etc. including but not limited to those discussed herein can be coupled with Bluetooth and/or ISM or other wireless frequencies can be used to communicate with the present invention. The present invention can take a number of actions including flashing lights, sounding alarms and alerts including but not limited to physical, electronic, wired, wireless, Web, text, e-mail, texting, placing automatically generated phone calls, contacting specified people, agencies, groups, services, departments, entities, individuals, etc. via web, mobile, smart, etc., cellular phones, tablets, other mobile devices, sending text message(s), etc., land line, conventional phones, e-mails, text messages, cellular services, etc., combinations of these, etc. In embodiments of the present invention, the absence of a signal, information, and/or response including but not limited to physiological including but not limited to blood pressure, heart rate, oxygen levels, insulin levels, temperature, other physiological monitors, sensors, etc., motion, proximity, temperature, humidity, room occupancy, room temperature, electrical power usage, lack of electrical power usage, water flow, water usage, gas usage, carbon monoxide and other gas sensing, lights and other appliances turned off or turned on (state of usage, time of usage, duration of usage), voice recognition, voice commands, sounds, movements, breakage, noise(s), patterns, etc., can trigger various responses including but not limited to turning off, dimming, sequencing, etc., lights, reducing temperature (i.e., turning off or setting to a lower temperature, heaters and furnaces), for example in the hot months and increasing temperature (i.e., turning off or setting the temperature of fans and air conditioners to a higher value), locking doors, etc.; or if someone is detected when no one is home turning on lights, flashing lights, flashing one or more colors of lights, setting certain lights to certain colors, sounding alarms, texting alarms, e-mailing alarms, sending alarm information to cell phones, smart phones, land lines, other devices and entities, including but not limited to friends, family, neighbors, first responders, medical personnel, fire department, police department, etc. Likewise, should a person be in trouble or should the present invention decide/believe/determine/etc. that a trouble condition exists, implementations of the present invention can turn on lights, flash lights, flash one or more colors of lights including, for example but not limited to red, set certain lights to certain colors, sound alarms, text alarms, e-mail alarms, send alarm information to cell phones, smart phones, land lines, other devices and entities, including but not limited to friends, family, neighbors, first responders, medical personnel, fire department, police department, etc. as well if a gas leak, water flow, flood condition, smoke, fire, etc. is detected.

For fluorescent tube direct replacements such as T8 linear fluorescent tubes, some embodiments may use an output lighting current in the range of approximately 130 mA or lower to, for example but not limited to, achieve constant current and output lumen performance over a large range of ballasts including, for example, 1, 2, 3 and 4 lamp ballasts that respectfully support 1, 2, 3 and 4 lamp outputs. Another example embodiment may use a output lighting current in the range of approximately 120 mA or 115 mA or lower for North American T8 32 watt and lower fluorescent lamps. In other implementations to be used elsewhere such as European versions other constant currents can be used as well as other current values for North American versions.

Turning to FIG. 11, a solid state fluorescent lamp replacement 1100 with external driver/converter/power supply 1112 etc. in accordance with some embodiments of the invention. Ballast interfaces 1104, 1108 connected to ballast bi-pin connections 1102, 1110, and can include circuitry for emulating fluorescent lamp heaters enabling fluorescent ballasts to function properly, switching/control circuitry to control output power based on presence, correct functioning, etc. of ballasts, etc. One or more external drivers/converters/power supplies 1112, etc. can receive power from the ballast interfaces 1104, 1108 to power an LED and/or OLED array 1106 or other lights in the solid state fluorescent lamp replacement 1100 as well as to optionally provide power to other external lights or devices that may be attached to the solid state fluorescent lamp replacement 1100 or positioned remotely. The ballast interfaces 1104, 1108 in some embodiments are rapid start ballast interfaces, although the solid state fluorescent lamp replacement 1100 is not limited to use with any particular type of ballast or to use with any ballast at all. The present invention can use linear regulation, switching regulation including but not limited to buck, buck-boost, boost-buck, boost, etc., transformer(s) with one or more secondaries, flyback(s) with one or more secondaries, switched capacitors, etc., combinations of these, etc. The rapid start ballast interface 1104, 1108 provides an appropriate emulation circuit or circuits for the heater/cathode connections of, for example, rapid start ballasts which could include but is not limited to resistors and capacitors and other passive and/or active components and parts.

The present invention provides a direct replacement for fluorescent tubes used in ballasts and permits dimming even/including if the ballast is not designed to support dimming Both wireless and wired control, dimming and monitoring can be accomplished with the present invention. For example the lighting system can use wired dimming using 0 to 10 V can be used or ISM, WiFi, Bluetooth, etc. The lighting system can use 0 to 10V or other analog, (e.g., 0 to 3V), 0 to 3V to 0 to 10V converters 0 to 10V to 0 to 3V converters, etc., DMX, DALI, RS232, RS422, RS485, USB, and other serial and/or parallel interfaces for communication, reporting, control, etc., and can use any suitable wireless, wired through connector or other means, connection. Some embodiments use an isolated interface.

Some embodiments of the present invention use, for example, but not limited to, a buck or boost or flyback or forward converter circuit that can be powered by AC lines (including universal voltage 80 to 305 VAC, 100 VAC, 120 VAC, 200 VAC, 220 VAC, 240 VAC, 277 VAC, 347 VAC, 480 VAC, etc. at, for example but not limited to, nominally 50/60 Hz) via, for example, but not limited to an EMI line filter that contains, for example, but not limited to inductors and which also can be powered by an electronic ballast that contains capacitors which limit/block/attenuate/etc. the 50/60 Hz line voltage and bypass (or put in parallel with, etc.) the EMI filter. For example, some embodiments of the present invention use one set of the two bi-pins to input AC line (i.e., 50 or 60 Hz, 47 to 63 Hz, etc.) power to implementations of the present invention while using at least one pin of both sets of the bi-pins to power the implementations of the present invention including the same implementations that are powered by AC from one set of the bi-pins from a ballast. In these type of embodiments, the AC input would appear across one set of bi-pins that would either be used for the heater in, for example but not limited to, electronic rapid start, prestart, programmed start, program start, dimmable, etc. or can be shorted out for, for example, but not limited to electronic instant start ballasts, etc. In some embodiments of the present invention, the tombstones can be adjusted to disconnect the connection between the bi-pins in the tombstone. In some embodiments of the present invention, the fluorescent lamp replacement (FLR) can be very different in form factor and even function from the fluorescent tube that is being replaced.

For example, turning to FIG. 12, in some embodiments a fluorescent fixture 1200 designed for four fluorescent tubes can receive a solid state fluorescent lamp replacement 1202 comprising a solid state light panel, such as an OLED panel, or a combination of one or more solid state light panels, an array of one or more solid state point light sources, or combinations of these. In some embodiments, light can be emitted from any surface or edge of the solid state fluorescent lamp replacement 1202, including the front face, rear face, edges, or any combinations of these. In some embodiments, multiple solid state FLR light sources are mounted in the fluorescent fixture 1200, including the illustrated solid state fluorescent lamp replacement 1202 and other light sources such as, but not limited to, solid state point or panel light sources mounted in the interior or exterior of the fluorescent fixture 1200. For example, the solid state fluorescent lamp replacement 1202 can produce direct or diffused light of one controllable color directly from the fluorescent fixture 1200, while another color or colors can be directed up or to the sides or in another direction from one or more other solid state point or panel light sources mounted in the interior or exterior of the fluorescent fixture 1200.

The diagram of FIG. 12 illustrates the solid state fluorescent lamp replacement 1202 as it is positioned to be inserted into an example illustrative fluorescent lamp fixture 1200. FIG. 13 illustrates the solid state fluorescent lamp replacement 1202 connected to the fluorescent fixture 1200. Again, in various embodiments any number of solid state fluorescent lamp replacement configurations can be used to replace any type or number of fluorescent lamps, in any type or configuration of fluorescent lamp fixture(s). Although a variety of example embodiments are shown in the Figures and described herein, the invention is not limited to any of these example embodiments.

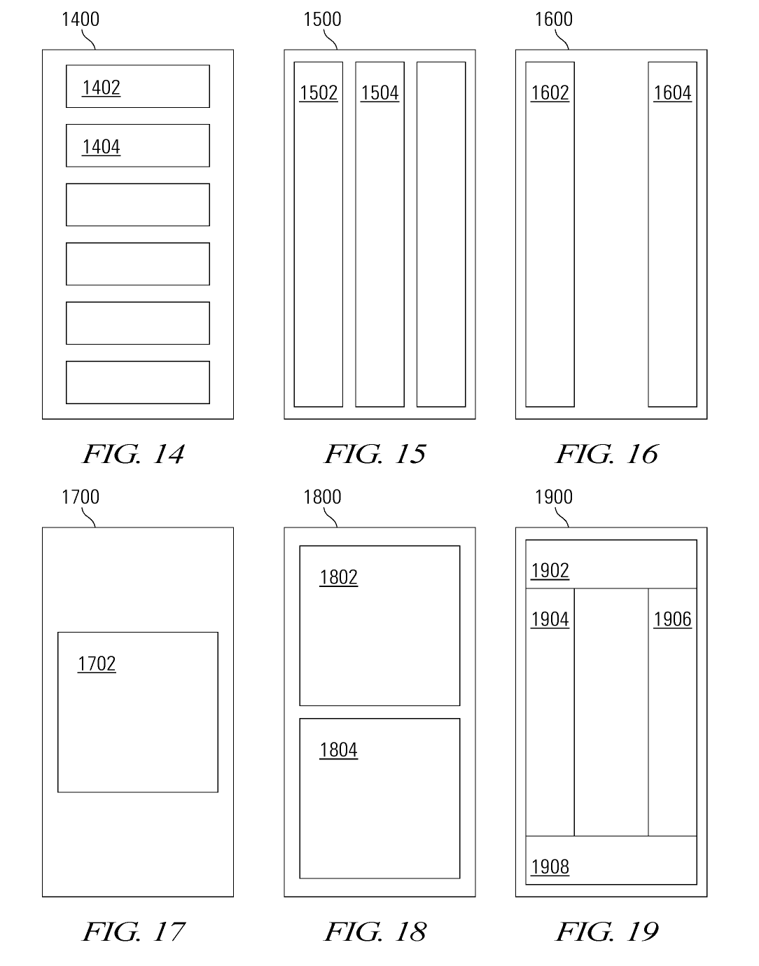

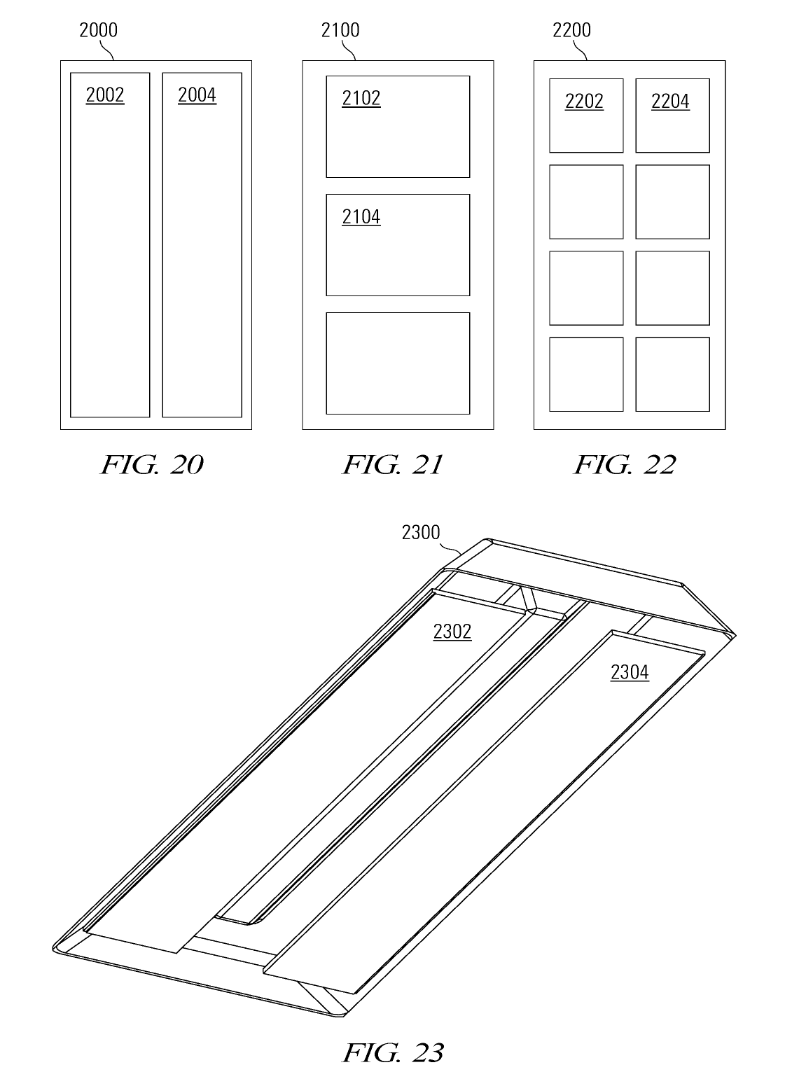

FIGS. 14-22 depict a variety of different configurations and arrangements of solid state fluorescent lamp replacements that can be used on fluorescent fixture, or of arrangements of solid state lights that can be mounted to any surface, stand, or other mounting arrangement and can be powered and controlled as disclosed herein. As shown in FIG. 14, some embodiments of a solid state lighting system 1400 include an array of linear solid state panel lights (e.g., 1402, 1404) arranged side to side across a short axis of the light. As shown in FIG. 15, some embodiments of a solid state lighting system 1500 include an array of linear solid state panel lights (e.g., 1502, 1504) arranged side to side across a long axis of the light. As shown in FIG. 16, in some embodiments of a solid state lighting system 1600 solid state panel lights (e.g., 1602, 1604) are spaced apart (with optional filler trim as desired for aesthetic purposes) or with smaller solid state light sources including LEDs, QDs, smaller OLED panels, etc. As shown in FIG. 17, in some embodiments of a solid state lighting system 1700 one solid state panel light (e.g., 1702) is provided (with optional filler trim as desired for aesthetic purposes) or with smaller solid state light sources including LEDs, QDs, smaller OLED panels, etc. As shown in FIG. 18, some embodiments of a solid state lighting system 1800 include an array of substantially square solid state panel lights (e.g., 1802, 1804). As shown in FIG. 19, some embodiments of a solid state lighting system 1900 include linear solid state panel lights (e.g., 1902, 1904, 1906, 1908) arranged in various directions or orientations (with optional filler trim as desired for aesthetic purposes). As shown in FIG. 20, some embodiments of a solid state lighting system 2000 include a pair of linear solid state panel lights (e.g., 2002, 2004) arranged side to side across a long axis of the light. As shown in FIG. 21, some embodiments of a solid state lighting system 2100 include three linear solid state panel lights (e.g., 2102, 2104). As shown in FIG. 22, some embodiments of a solid state lighting system 2200 include a two-dimensional array of solid state panel lights (e.g., 2202, 2204). Although shown and depicted as squares in many of the figures, implementations of the present invention can take many forms and form factors and essentially can be virtually any form factor or shape from simple to complex, from triangle to parallel piped, to circular, to spherical, to elliptical, to parabolic, etc.

The present invention can be dimmable powered on the AC lines or the ballast. The present invention can work with dimmable ballasts of any type including but not limited to 0 to 10 V, DALI, TRIAC, and powerline control (PLC), etc., instant-start ballasts, rapid start ballasts, programmed start ballasts, programmable start ballasts, pre-start ballasts, magnetic ballasts, and essentially any type of ballast. The present invention can use a switch, including a momentary switch, or other types of mechanical, electrical, electromechanical, etc. plug in, connections, including plug-in safety connectors for shock hazard protection.

For example a momentary switch can be depressed to complete a circuit that allows the ballast to power the present invention once the momentary switch is released. Should a potential shock hazard exist the circuit would not latch and until the shock hazard is eliminated, pushing the momentary button would not latch and activate the circuit. The present invention can also use remote enabling to provide protection including protection from shock hazard by essentially keeping the ballast turned off and in a high impedance state until remote commanded (i.e., by remote control, smart phone, tablet, computer, other device, user input, controls/buttons/etc. on the implementations, etc.) to disable the protection/shock hazard. In some embodiments, the user will need to request to disable the protection/shock hazard and then verify/confirm that request to actually disable.

The present invention can use wireless control to control the dimming level of the lighting, etc. The present invention allows for full spectrum, including full visible spectrum lighting and control, dimming and/or monitoring including red, green, blue (RGB); red, green, blue, amber, (RGBA); red, green, blue, white (RGBW), red, green, blue, amber, white (RGBAW), more than one white color temperature (RGBWW, RGBAWW, RGBAWWW, GBAWW, etc.), additional or fewer colors/wavelengths, etc., combinations of these, etc.

The present invention can use small cards, memories, etc. that can consist of any type of semiconductor memory, magnetic memory, ferromagnetic memory, optical memory, or other memory, RFID, etc., including but not limited to FLASH memory, non-volatile memory, EEPROM, EPROM, PROM, AND memory, OR memory, etc. Such memory can be used to provide programmable information including, for example, but not limited to, name to be used for the present invention, address, individual address, group address, location, properties, behavior, pre-programmed features, data logging, storage of audio and or video information, etc., communications, encryption, type, security, etc.

The present invention, in addition to providing analog and/or digital interfaces for control (including dimming and monitoring, logging, etc.) can also provide isolated (or non-isolated) power derived from, for example, but not limited to, the ballast itself. An example would be to take current/power from the ballast by rectifying the AC output from the ballast and filtering as desired. Example embodiments which are not intended to be limiting in any way or form include using forward converters or flyback converters for isolated power, using buck, boost, buck-boost, boost-buck, of any type, for, topology, parts, components, including one or more of these switching/storage circuits, elements, topologies, etc., linear regulators including current regulators, etc., extra windings including but not limited to one or more of those discussed herein—etc. In some embodiments of the present invention, a keep-alive circuit is used when the present invention is dimmed to very low levels or off. Non-isolated supplies can use isolated galvanic to provide isolation for example with buck-boost, buck, boost-buck, boost etc. topologies including one or more tagalong inductors such as those disclosed in U.S. patent application Ser. No. 13/674,072, filed Nov. 11, 2012 by Sadwick et al. for a “Dimmable LED Driver with Multiple Power Sources”, which is incorporated herein for all purposes, and which may be used and incorporated into embodiments of the present invention.

The present invention can work with all types of sensors and controls including ones that sense movement, proximity, light, solar light, solar energy, daylight, light spectrum(s), temperature, time of day, location, presence, mechanical, electronic, electrical, sound, vibration, words, voice, voice commands, voice recognition, cell phones, smart phones, tablets, computers, servers, WiFi, Bluetooth, IEEE 802, ISM, USB, serial and/or parallel communications, RFID, entry cards, access cards, signal strength, etc. The present invention can also be used in simple and/or autonomous control and associated modes. Some implementations may require no external controller or a very simple, easy to use, intuitive one, etc. In some embodiments, the lighting system supports multiple levels of user control and sophistication, complexity, for example selectably hiding or making available interfaces that provide for simply turning on or off lights, dimming, controlling color, programming these functions, providing remote monitoring of input power conditions, ballast functioning, output power to lights, ambient conditions, as well as all other sensing/monitoring/reporting functions disclosed herein. Such multiple levels of user control and complexity can be configured with multiple operating modes allowing for simple control only, for full control and monitoring of the system, or for a number of configurable modes in between to allow access to only select control and monitoring functions.

Various embodiments of the lighting systems can include one or more types of sensors, allowing for control of the light output as well as for reporting ambient conditions to a user. An example of an optical spectrum analyzer can consist of optical sensors and detectors that are wavelength/frequency/color specific and can be stacked either vertically (i.e., layered/stacked on top of each other) or horizontally (stacked side by side, etc.). Such detectors/sensors could be measured using current or voltage sensitive circuits that are fed or multiplexed to one or more analog to digital converters (ADCs) that can also be used to provide either analog or digital (including but not limited to digital to analog converters ADCs) (or both) feedback/control/readout/etc. to/for the present invention or amplifiers including logarithmic amplifiers, current amplifiers, charge amplifiers, voltage amplifiers, operation amplifiers or any type or form, combinations of these, etc. Such sensors/detectors can be arrayed or act separately/independently including but not limited to being separately addressed and read to control/feedback the intensity/color/wavelength/frequency levels, etc.

The present invention can also use wired and/or wireless interfaces including but not limited to serial interfaces including but not limited to those discussed herein to, for example, program/set/assign/etc., the address, name, identification, identifier, grouping, group, etc. Such setting/assignments/etc., can be also done/accomplished/performed by the user and be user-programmed, etc. The example serial port, for example, USB port can be used for other purposes including direct communications with the present device, reprogramming the parts (or all) of the firmware/software of the present invention, charging other devices using the example USB port, etc. Such other devices could include but are not limited to cell phones, smart phones, tablets, computers, batteries, other energy storage devices, other personal assistant devices, sensors and detectors, portable lighting, etc.

The present invention can be powered by a ballast in a number of ways including both magnetic and electronic ballasts including electronic ballasts that are instant-start, rapid start, programmable start, dimmable, etc. The ballast output(s) can be combined/connected as needed to achieve the needed/desired performance.

Some embodiments of the present invention can also be used to detect the presence (or absence) of a persons or persons including whether a person or persons are spending too much time or too little time in a particular location and, in some embodiments, automatically alert and provide alerts via, for example, but not limited to, e-mail, phone calls, web messages, text messages, etc.

The present invention can have current and/or voltage control or both including with automatic switchover or automatic crossover from voltage to current control or current to voltage control and/over power control. The setpoints, parameters, conditional statements, etc. be manually set, factory set, user set, remote control set using, for example, wired or wireless control, monitoring, communications, etc. The control can be local, fixed or remotely programmed and set. Wired control can include but is not limited to 0 to 10V, 1 to 8 V, 0 to 5 V, 0 to 3V, 0 to 10 V, etc., SPI, USB, powerline control, I2C, serial, SPC., etc. Wireless control can include but is not limited to ZigBee, Bluetooth, IEEE 802, WiFi, ISM, RF, IR, infrared, IrDA, infrared modulated control (i., 30 to 56 kHz), RFID, ZWave, etc.

Solid state lighting panels can be mounted in fluorescent lamp fixtures either in fixed immovable fashion or can be mounted on movable mounts allowing panels or light sources to be directed, aimed, tilted, pivoted, rotated, etc. Such angling/tilting/etc. can be fixed, manually adjustable, automatically adjustable, program adjustable, remotely adjustable, etc. Such tilted/angled panels, tiles, etc. can use motors, levers, hinges, flaps, wires, etc. and can be set, programmed, moved, adjusted, etc. by virtually any type of stimuli and/or input, including but not limited to time of day, light level, motion, occupancy, location, weather, Sun phase and cycle, circadian rhythm phase, direction, temperature, day of the week, date, etc., weather, personal information, health conditions and status including illnesses, diseases, chronic health conditions and problems, medical treatments, wearable devices, health status, etc., combinations of these, sequencing of these, etc.

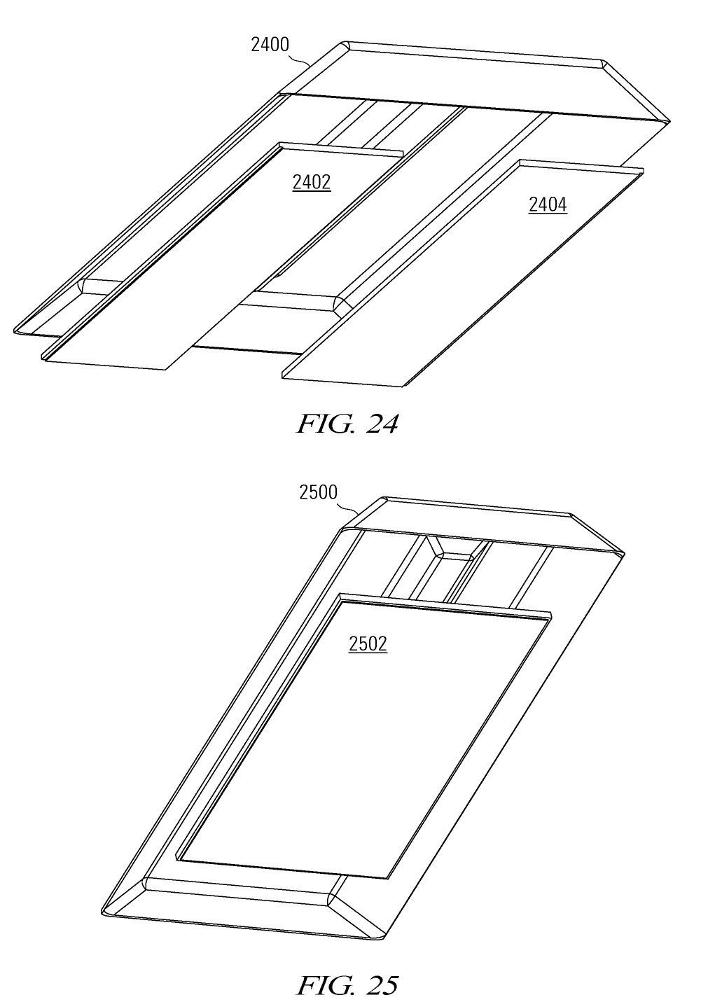

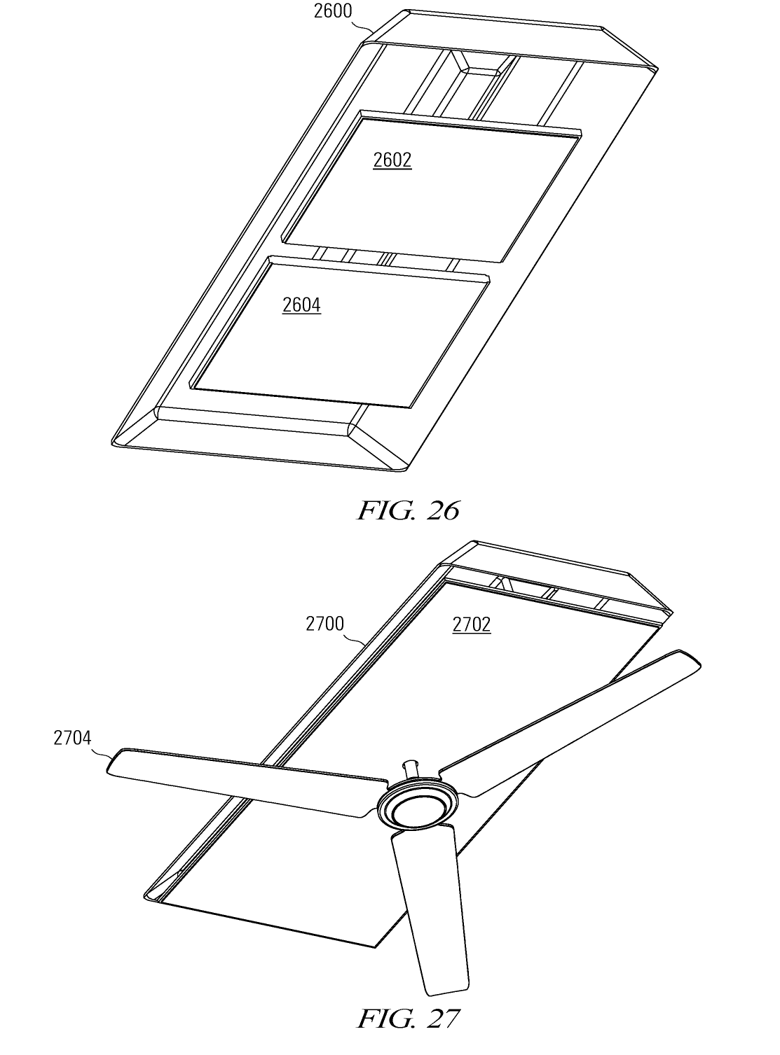

Some example embodiments of solid state lighting panels mounted in fluorescent lamp fixtures are depicted in FIGS. 23-26. Turning to FIG. 23, two solid state lighting panels 2302, 2304 are shown mounted in a fluorescent lamp fixture 2300. Turning to FIG. 24, two solid state lighting panels 2402, 2404 are shown as they appear mounted in a fluorescent lamp fixture 2400 on a mounting system which, for example, but not limited to, allows them to extend somewhat from the fluorescent lamp fixture 2400, for example on pivoting mounts such that the solid state lighting panels 2402, 2404 can be angled or moved, either automatically in motorized fashion or manually. The two solid state lighting panels 2402, 2404 are also shown as they would appear adjacent a fluorescent lamp fixture 2400 immediately prior to mounting in fixed fashion. Turning to FIG. 25, a solid state lighting panel 2502 is shown mounted in a fluorescent lamp fixture 2500. Turning to FIG. 26, two solid state lighting panels 2602, 2604 are shown mounted in a fluorescent lamp fixture 2600. FIG. 24 and associated figures are intended to be examples only of the present invention and in no way or form limiting.

The present invention can have current and/or voltage control or both including with automatic switchover or automatic crossover from voltage to current control or current to voltage control and/over power control. The setpoints, parameters, conditional statements, etc. be manually set, factory set, user set, remote control set using, for example, wired or wireless control, monitoring, communications, etc. The control can be local, fixed or remotely programmed and set. Wired control can include but is not limited to 0 to 10V, 1 to 8V, 0 to 5V, 0 to 3V, 0 to 10V, etc., SPI, USB, powerline control, I2C, serial, SPC., etc. Wireless control can include but is not limited to ZigBee, Bluetooth, IEEE 802, WiFi, ISM, RF, IR, infrared, IrDA, infrared modulated control (i.e., 30 to 56 kHz), RFID, ZWave, etc.

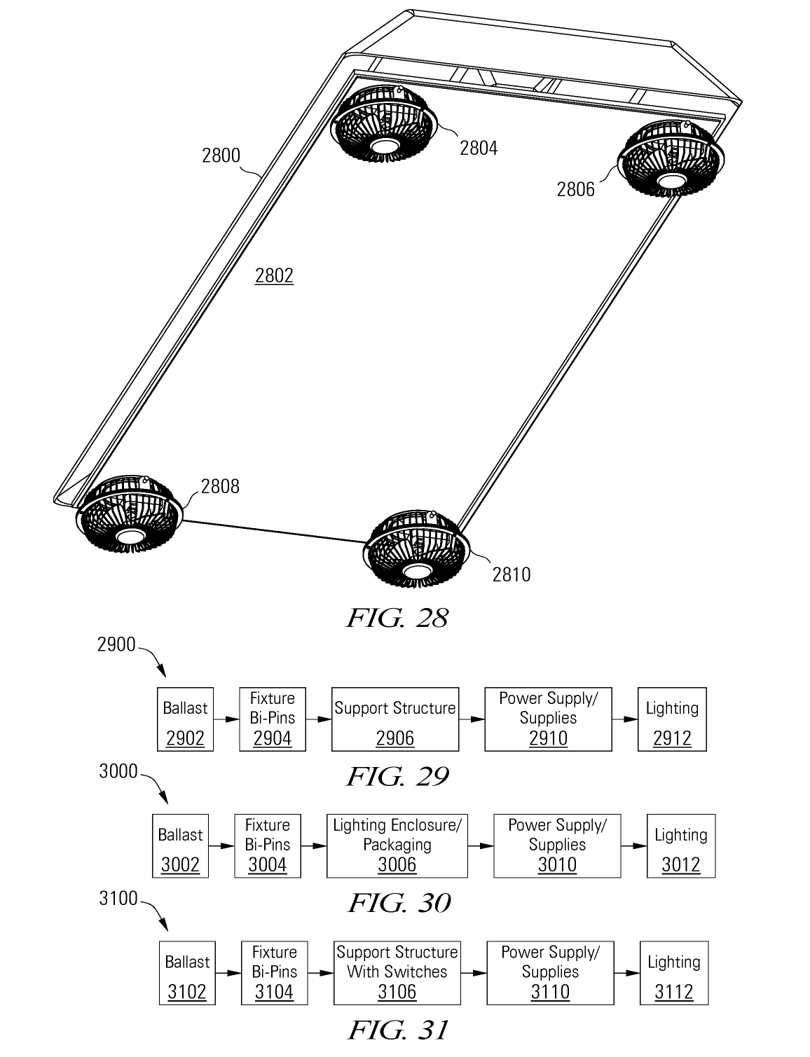

Turning to FIGS. 27-28, additional devices such as fans can also be included in the lighting systems disclosed herein, and can be powered through ballasts if any are present. Any types of fans or other devices can be included in and powered by lighting systems. As shown in FIG. 27, a ceiling fan 2704 is included with a solid state lighting panel 2702 in a fluorescent lamp fixture 2700. As shown in FIG. 28, multiple fans 2804, 2806, 2808, 2810 can be included with a solid state lighting panel 2802 in a fluorescent lamp fixture 2800. The number and types of fans or other devices that can be included in the lighting systems disclosed herein is not limited to the example embodiments illustrated in the Figures. The fans can also contain/include SSL/LED lighting.

In addition to the fans depicted in the figures and discussed herein, motorized track lighting and other lighting including but not limited to PAR, MR16, GU10, track lighting, magnetic low voltage lighting, lighting discussed herein, etc. may be used such that the motor(s) can be controlled locally and/or remotely. Such motors and related devices and components, etc. can also be used to tilt, move, extend, articulate, direct, swing out, etc. the lighting and, for example, the fans and other accessories, etc. Some embodiments of the present invention can also have DC to AC inverters that, for example, provide 50 or 60 Hz AC voltage (e.g., 120 V AC or 220 V AC) that can also be selected.

The Figures show some examples of implementations of the present invention in which the fluorescent lamps have been replaced by the present invention that, for example, consists of a power supply or supplies that are powered by the ballast and provide conventional and new lighting designs, capabilities, form factors, etc. which can be both retrofitted into existing fixtures and luminaires as well, for example, into new construction, etc. The Figures are intended to be examples and in no way or form should be considered to be limiting of the present invention. The present invention can be supported in a number of ways including being supported by the electrical bi-pin connectors of the fixture or luminaire, by inserting other supports and structures, by using magnets, by using screws, tape, double sided tape, etc. The structures may also be illuminated by lighting including, but not limited to, OLEDs and/or LEDs, QDs, other types of solid state lighting, etc. which can be, for example, white, RGB, RGBW, RGBAW, RGBAWW, etc. along with and, for example, various other types of functions and applications including those that provide/require electrical energy, mechanical strength, energy harvesting, solar detection, solar energy, daylighting harvesting, motion sensing, infrared sensing, spectrum sensing/detection, proximity detection/sensing, other sensors and detectors including but not limited to those discussed herein, etc. Notably, in some embodiments of the present invention the ballast bi-pins may be connected to the support structure so as to provide a path including an electrically safe path that allows electrical connection between the ballast and the power supply and the lighting that is powered by the power supply. This permits a number of advantageous features and functions including replacement/interchangeability of the power supply and/or the lighting, safe connections, shock hazard protection, etc. Other implementations of the present invention can also use the fixture via screws, clamps, wiring, sticky tape, double-sided tape, 3M ‘Contact’, etc., to support the power supplies and solid state lighting including but not limited to FLRs and other power consuming/using devices, electronics, etc.

Some example embodiments of the present invention using a ballast are depicted in FIGS. 29-31. Turning to FIG. 29, one example of a lighting system 2900 includes a ballast 2902 connected to fixture bi-pins 2904 which are mounted in a support structure 2906. A power supply or supplies 2910 draw power from the bi-pins 2904 to power the lighting 2912 and, optionally, other devices. Turning to FIG. 30, another example of a lighting system 3000 includes a ballast 3002 connected to fixture bi-pins 3004 which are mounted in a lighting enclosure or other packaging or housing 3006. A power supply or supplies 3010 draw power from the bi-pins 3004 to power the lighting 3012 and, optionally, other devices. Turning to FIG. 31, another example of a lighting system 3100 includes a ballast 3102 connected to fixture bi-pins 3104 which are connected through a support structure with switches 3106, providing shock hazard protection particularly during installation or to control output power. A power supply or supplies 3110 draw power from the bi-pins 3104 to power the lighting 3112 and, optionally, other devices. Again, these Figures are intended to be example embodiments of the present invention and in no way or form limiting.

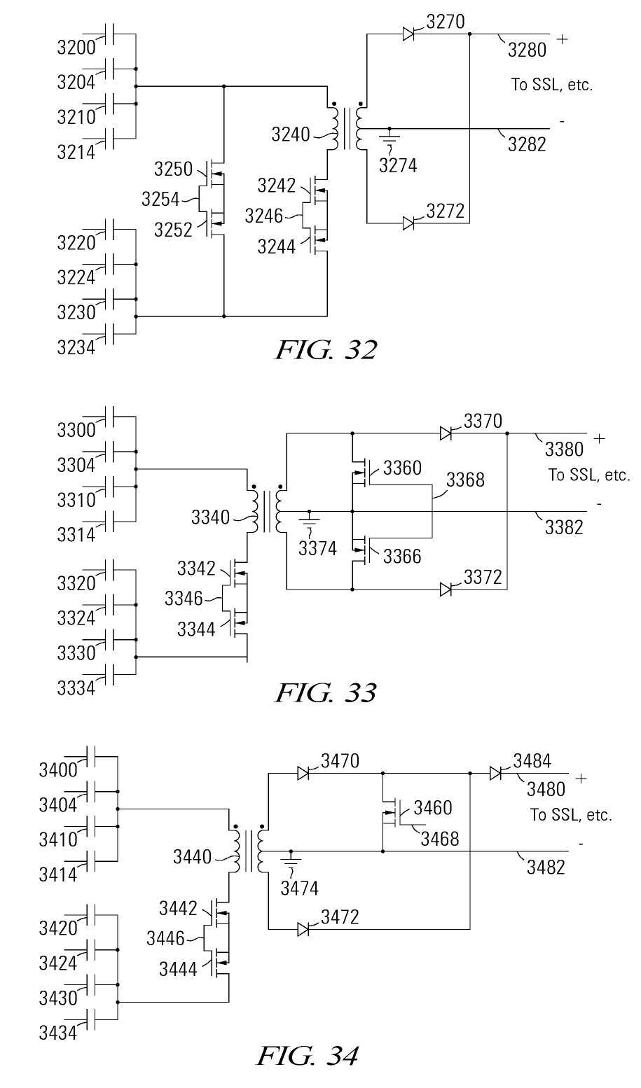

Turning now to FIGS. 32-38, example power supplies for solid state fluorescent lamp replacement lighting systems are depicted in accordance with some embodiments of the present invention. A ballast (not shown) provides electrical power at the left side of each of the power supplies of FIGS. 32-38. Each of the ballast lamp outputs are respectively connected to capacitor pairs. For example, in an embodiment with four ballast lamp outputs as in a fluorescent lamp fixture configured to hold four fluorescent tubes, as shown in FIG. 32, one ballast lamp output is connected across capacitors 3200 and 3220, a second ballast lamp output is connected across capacitors 3204 and 3224, a third ballast lamp output is connected across capacitors 3210 and 3230, and a fourth ballast lamp output is connected across capacitors 3214 and 3234. The input capacitors are in turn connected to, for example, one or more transformers 3240 (in many applications and implementations, only one transformer is needed/used; in some embodiments of the present invention, capacitors may be optional). Although shown as a center tapped transformer 3240 in FIGS. 32-38, non-center tapped transformers and, in general, any type of transformer may be used including ones that require full bridge rectifiers, synchronous rectifiers, silicon controlled rectifiers, etc. The center tap can be grounded 3274 or can be floating as a local ground node or reference node.

In other embodiments, flyback transformer(s) may be used. Additional primary, secondary and other windings may be added/included/used/etc. in and with the present invention. In FIGS. 32-38, the rectified secondary output 3280, 3282, for example from diodes 3270, 3272, for example but not limited to, is then fed to the lighting or additional circuitry, including for example, current control and/or voltage control, etc. The present invention can have one or more (or all) of shock protection, over current protection, over voltage protection, over temperature protection, short circuit protection, etc., combinations of these, etc. The present invention can use/have more than one color or more colors than just white or any color temperature of white including but not limited to, red green blue, red green blue amber, red green blue amber white, more than one white color temperature, etc., combinations of these, etc. Switches or transistors 3250, 3252 are used to provide current control (or, in some embodiments, voltage control or both) by shunting excess current from reaching the primary of the transformer 3240, with any suitable control circuit controlling the gates 3254. Switches or transistors 3242, 3244 provide shock hazard protection by effectively opening up the primary of the transformer 3240 thus not permitting current to flow. In these embodiments the AC output of the ballast provides the input for the transformer. In many embodiments, switches 3250 and 3252 are also open to not allow current flow and, as with switches 3242, 3244 provide shock hazard protection. As the electronic ballast typically puts out high frequencies often higher than 30 kHz to 40 kHz, the transformer(s) can be compact in size, weight, form factor, etc. Transistors 3242, 3244 are configured in a back to back configuration with common gates and sources. Shock hazard protection can be implemented by mechanical, electrical, vibration, other, etc., combinations of these, etc. means. The gates 3246 of protection transistors 3242, 3244 can thus be controlled in any suitable manner, for example by sensing circuits, manually operated switches, automatic switches, or any other means for insuring, detecting, permitting, etc. when the solid state fluorescent lamp replacement has been correctly installed in a fluorescent lamp fixture, such as, but not limited to, the means disclosed in PCT patent application PCT/US14/63596, filed Oct. 31, 2014 for “Fluorescent Lamp Replacement LED Protection”, which is hereby incorporated by reference for all purposes. In some embodiments of the present invention, the wired or wireless remote connections may be used to ‘unlock’ and turn off the protection and enable the turn on of the light source.

Turning now to FIG. 33, another example power supply 3300 for solid state fluorescent lamp replacement lighting systems is depicted in accordance with some embodiments of the present invention. In this embodiment, the current (or voltage) control is accomplished by using switches represented as two transistors 3360, 3366 with common gates 3368 which each could consist of, for example, but not limited to, two or more back-to-back transistors including but not limited to MOSFETs connected such that the gates are connected together and sources are connected together, respectively, or BJTs with the bases connected together and emitters connected together, respectively, to shunt/short the excess current on the secondary or secondaries of the transformer 3340 before the excess current reaches (or voltage builds up at) either diode 3370 or 3372. Each of the ballast lamp outputs are respectively connected to capacitor pairs. For example, in an embodiment with four ballast lamp outputs as in a fluorescent lamp fixture configured to hold (up to) four fluorescent tubes, as shown in FIG. 33, one ballast lamp output is connected across capacitors 3300 and 3320, a second ballast lamp output is connected across capacitors 3304 and 3324, a third ballast lamp output is connected across capacitors 3310 and 3330, and a fourth ballast lamp output is connected across capacitors 3314 and 3334. The input capacitors are in turn connected to, for example, one or more transformers 3340 (in many applications and implementations, only one transformer is needed/used, however more than one transformer can be used; in some embodiments of the present invention, capacitors may be optional). Again, type of transformer may be used including ones that require full bridge rectifiers, synchronous rectifiers, silicon controlled rectifiers, etc. The center tap can be grounded 3374 or can be floating as a local ground node or reference node. In some embodiments, the number of outputs connected to implementations of the present invention may be less than the total number of outputs and possible total number of lamps that the ballast can support.

The rectified secondary output 3380, 3382, for example from diodes 3370, 3372, for example but not limited to, is then fed to the lighting or additional circuitry, including for example, current control and/or voltage control, etc. or to power other types of devices including but not limited to fans, blowers, heaters, other types of lights, laptops, USB connectors/interfaces/cameras, etc., combinations of these as well as other items, devices, things, etc. discussed herein. Switches or transistors 3342, 3344 provide shock hazard protection by effectively opening up the primary of the transformer 3340 thus not permitting current to flow, controlled through common gates 3346 in any suitable manner as disclosed above.

Turning now to FIG. 34, another example power supply 3400 for solid state fluorescent lamp replacement lighting systems is depicted in accordance with some embodiments of the present invention. In this embodiment, the current from the secondary or secondaries of the transformer 3440 passes through diodes 3470 or 3472 and then, for example, but not limited to, the excess current is shunted/shorted via switch/transistor 3460, controlled via gate 3468 in any suitable manner, such than when transistor 3460 is turned on the current is shunted/shorted through transistor 3460 and does not pass through diode 3484. Diode 3484 also prevents the load or further circuits/electronics on the cathode side of diode 3484 from being shorted when transistor 3460 is turned on.

Each of the ballast lamp outputs are respectively connected to capacitor pairs. For example, in an embodiment with four ballast lamp outputs as in a fluorescent lamp fixture configured to hold four fluorescent tubes, as shown in FIG. 34, one ballast lamp output is connected across capacitors 3400 and 3420, a second ballast lamp output is connected across capacitors 3404 and 3424, a third ballast lamp output is connected across capacitors 3410 and 3430, and a fourth ballast lamp output is connected across capacitors 3414 and 3434. The input capacitors are in turn connected to, for example, one or more transformers 3440 (in many applications and implementations, only one transformer is needed/used, however more than one and more transformers than the number of lamp outputs of the ballast may be used; in some embodiments of the present invention, capacitors may be optional). Again, type of transformer may be used including ones that require full bridge rectifiers, synchronous rectifiers, silicon controlled rectifiers, etc. The center tap can be grounded 3474 or can be floating as a local ground node or reference node. Again, this and the other figures are intended to be examples and not limiting in any way or form.

The rectified secondary output 3480, 3482, for example from diodes 3470, 3472, for example but not limited to, is then fed to the lighting or additional circuitry, including for example, current control and/or voltage control, etc. Switches or transistors 3442, 3444 provide shock hazard protection by effectively opening up the primary of the transformer 3440 thus not permitting current to flow through the transformer and to the rest of the present invention, controlled through common gates 3446 in any suitable manner as disclosed above.

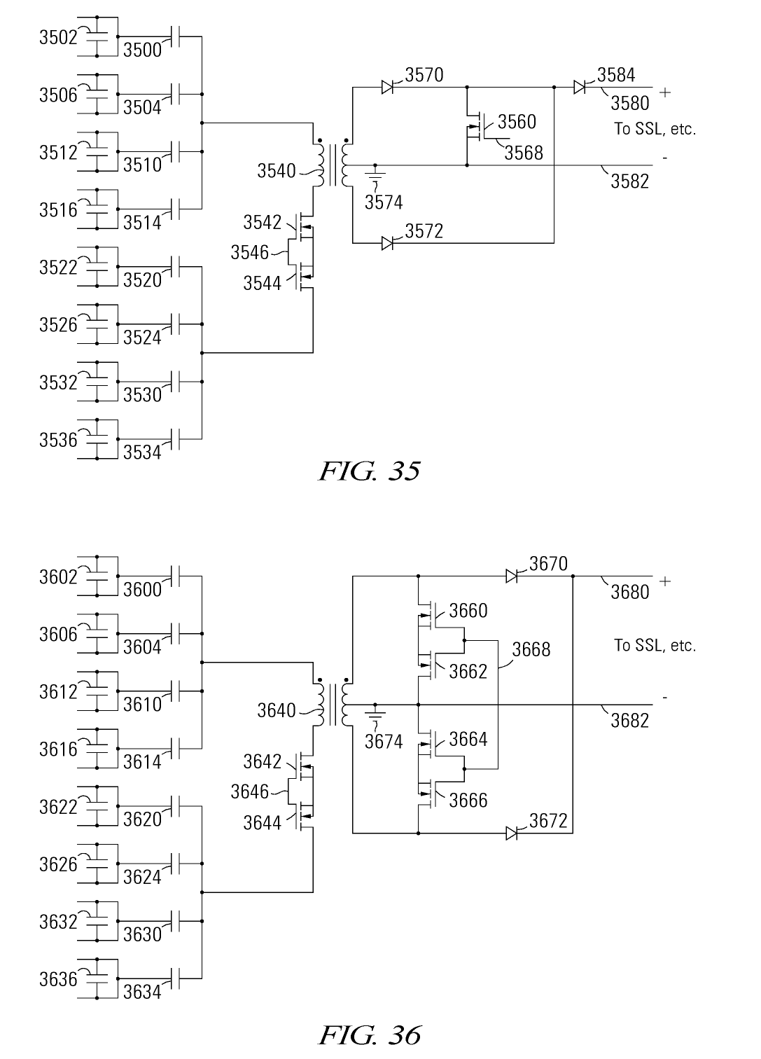

Turning now to FIG. 35, another example power supply 3500 for solid state fluorescent lamp replacement lighting systems is depicted in accordance with some embodiments of the present invention. In this embodiment, the current from the secondary or secondaries of the transformer 3540 passes through diodes 3570 or 3572 and then the excess current is shunted/shorted via switch/transistor 3560, controlled via gate 3568 in any suitable manner, such than when transistor 3560 is turned on the current is shunted/shorted through transistor 3560 and does not pass through diode 3584. Diode 3584 also prevents the load or further circuits/electronics on the cathode side of diode 3584 from being shorted when transistor 3560 is turned on.

In the embodiment of FIG. 35, capacitor pairs 3502 and 3522, 3506 and 3526, 3512 and 3532, 3516 and 3536 represent or comprise circuits capable of emulating the heater/cathode of a fluorescent lamp and are intended to be illustrative and not limiting in any way or form and not representative of other embodiments and/or implementations of the present invention) thus permitting rapid start, programmable start, programmed start, dimmable ballasts, etc. ballasts to work and be compatible with such ballasts as well as instant-start ballasts. For instant start ballasts, the capacitors 3502 and 3522, 3506 and 3526, 3512 and 3532, 3516 and 3536 may be sufficient without additional circuits, components, parts, sub-circuits, etc. with passives and/or active elements, however additional passive and active components including but not limited to additional capacitors, resistors, inductors, transistors, ICs, etc., combinations of these, etc. may be incorporated and included in the heater emulation or elsewhere in the present invention. Each of the capacitors 3502 and 3522, 3506 and 3526, 3512 and 3532, 3516 and 3536 is connected across a pair of bi-pin outputs from one of the eight ballast connections provided for the two ends of four fluorescent lamps being replaced in this example embodiment being depicted. Of course if the ballast had less than 4 outputs for four fluorescent lamps, then there would be fewer sets of capacitors and other related components, etc.; if there were more than four sets of outputs to power more than four fluorescent tubes then there could be more than 4 sets of capacitors, etc.

Each of the emulation circuit outputs are respectively connected to capacitor pairs. For example, in an embodiment with four ballast lamp outputs as in a fluorescent lamp fixture configured to hold four fluorescent tubes, as shown in FIG. 35, one ballast lamp output is connected across capacitors 3500 and 3520, a second ballast lamp output is connected across capacitors 3504 and 3524, a third ballast lamp output is connected across capacitors 3510 and 3530, and a fourth ballast lamp output is connected across capacitors 3514 and 3534. The input capacitors are in turn connected to, for example, one or more transformers 3540 (in many applications and implementations, only one transformer is needed/used, however more than one and more transformers than the number of lamp outputs of the ballast may be used; in some embodiments of the present invention, capacitors may be optional). Again, essentially any type, topology, design, etc. of transformer may be used including ones that require full bridge rectifiers, synchronous rectifiers, silicon controlled rectifiers, etc. The center tap can be grounded 3574 or can be floating as a local ground node or reference node.

The rectified secondary output 3580, 3582, for example from diodes 3570, 3572, for example but not limited to, is then fed to the lighting or additional circuitry, including for example, current control and/or voltage control, etc. Switches or transistors 3542, 3544 provide shock hazard protection by effectively opening up the primary of the transformer 3540 thus not permitting current to flow, controlled through common gates 3546 in any suitable manner as disclosed above.

Turning now to FIG. 36, another example power supply 3600 for solid state fluorescent lamp replacement lighting systems is depicted in accordance with some embodiments of the present invention. In this embodiment, a back-to-back set of MOSFET switches configured in pairs (transistors 3660 and 3662, 3664 and 3666) provide a controllable shunt/shorting path for certain embodiments of the present invention, controlled in any suitable manner via common gates 3668.

The current from the secondary or secondaries of the transformer 3640 passes through diodes 3670 or 3672 and then the excess current is shunted/shorted via switch/transistor 3660, controlled via gate 3668 in any suitable manner, such than when transistor 3660 is turned on the current is shunted/shorted through transistor 3660 and does not pass through diode 3684. Diode 3684 also prevents the load or further circuits/electronics on the cathode side of diode 3684 from being shorted when transistor 3660 is turned on.

Capacitor pairs 3602 and 3622, 3606 and 3626, 3612 and 3632, 3616 and 3636 represent or comprise circuits capable of emulating the heater/cathode of a fluorescent lamp thus permitting rapid start, programmable start, programmed start, dimmable ballasts, etc. ballasts to work and be compatible with such ballasts as well as instant-start ballasts. For instant start ballasts, the capacitors 3602 and 3622, 3606 and 3626, 3612 and 3632, 3616 and 3636 may be sufficient without additional circuits, components, parts, sub-circuits, etc. with passives and/or active elements. Each of the capacitors 3602 and 3622, 3606 and 3626, 3612 and 3632, 3616 and 3636 is connected across a pair of bi-pin outputs from one of the eight ballast connections provided for the two ends of four fluorescent lamps being replaced.

Each of the emulation circuit outputs are respectively connected to capacitor pairs. For example, in an embodiment with four ballast lamp outputs as in a fluorescent lamp fixture configured to hold four fluorescent tubes, as shown in FIG. 36, one ballast lamp output is connected across capacitors 3600 and 3620, a second ballast lamp output is connected across capacitors 3604 and 3624, a third ballast lamp output is connected across capacitors 3610 and 3630, and a fourth ballast lamp output is connected across capacitors 3614 and 3634. The input capacitors are in turn connected to, for example, one or more transformers 3640 (in many applications and implementations, only one transformer is needed/used; in some embodiments of the present invention; however more than one transformer may be used, capacitors may be optional). Again, the type or types of transformer or transformers may be used including ones that can use or require full bridge rectifiers, synchronous rectifiers, silicon controlled rectifiers, etc. The center tap can be grounded 3674 or can be floating as a local ground node or reference node.

The rectified secondary output 3680, 3682, for example from diodes 3670, 3672, for example but not limited to, is then fed to the lighting or additional circuitry, including for example, current control and/or voltage control, etc. and to other types of power consuming or power converting, inverting, etc. devices, circuits, units, systems, etc. including without limitation or limit, ones discussed herein. Switches or transistors 3642, 3644 provide shock hazard protection by effectively opening up the primary of the transformer 3640 thus not permitting current to flow, controlled through common gates 3646 in any suitable manner as disclosed above.

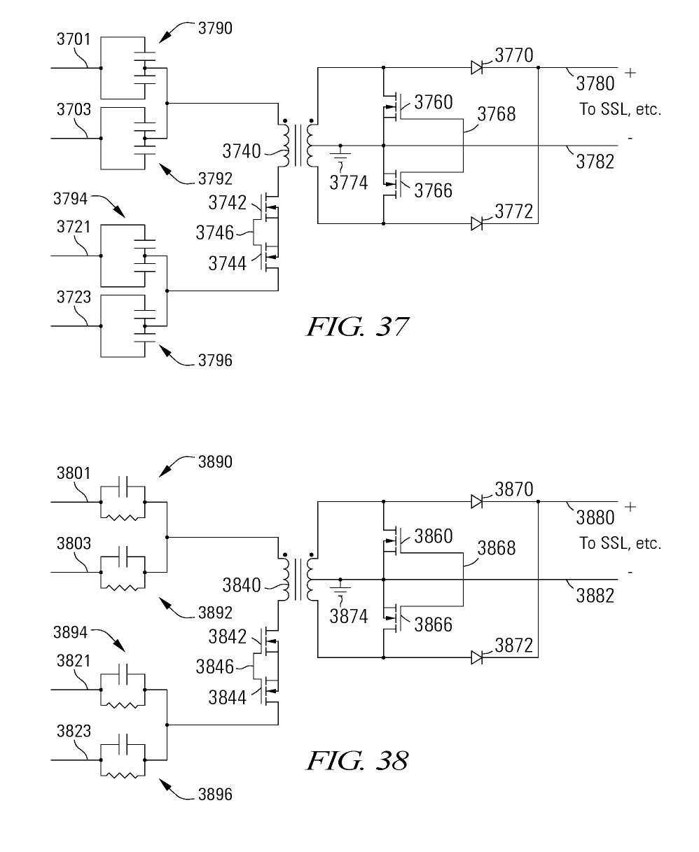

FIGS. 37-38 depict other embodiments of heater emulation circuits, showing the bi-pin ballast output connections 3701, 3703, 3721, 3723 for only one fluorescent lamp for clarity. In the example embodiment of FIG. 37, heater emulation circuits 3790, 3792, 3794, 3796 comprise parallel sets of capacitors. In the example embodiment of FIG. 38, heater emulation circuits 3890, 3892, 3894, 3896 connected to bi-pin ballast output connections 3801, 3803, 3821, 3823 comprise parallel connected capacitor and resistor pairs. Such illustrative depictions are to be construed as examples and in no way or form are to be construed as limiting in any of the heater emulation circuits and other associated elements, parts, etc. of embodiments of the present invention. Other embodiments of the present invention may incorporate/use/contain/etc. other passive and active components including switches as part of the heater emulation and other parts of the present invention.

Turning to FIG. 37, the current (or voltage) control is accomplished by using switches represented by in the figure below as two transistors 3760, 3766 with common gates 3768 which each could consist of, for example, but not limited to, two or more back-to-back transistors including but not limited to MOSFETs connected such that the gates are connected together and sources are connected together, respectively, or BJTs with the bases connected together and emitters connected together, respectively, to shunt/short the excess current on the secondary or secondaries of the transformer 3740 before the excess current reaches (or voltage builds up at) either diode 3770 or 3772. Each of the ballast lamp outputs are respectively connected to capacitor pairs. For example, in an embodiment with four ballast lamp outputs as in a fluorescent lamp fixture configured to hold four fluorescent tubes, as shown in FIG. 37, one ballast lamp output is connected across capacitors 3700 and 3720, a second ballast lamp output is connected across capacitors 3704 and 3724, a third ballast lamp output is connected across capacitors 3710 and 3730, and a fourth ballast lamp output is connected across capacitors 3714 and 3734. The input capacitors are in turn connected to, for example, one or more transformers 3740 (in many applications and implementations, only one transformer is needed/used; although more can be used if desired, needed, etc.; in some embodiments of the present invention, capacitors may be optional). Again, type of transformer may be used including ones that require full bridge rectifiers, synchronous rectifiers, silicon controlled rectifiers, etc. The center tap can be grounded 3774 or can be floating as a local ground node or reference node.

The rectified secondary output 3780, 3782, for example from diodes 3770, 3772, for example but not limited to, is then fed to the lighting or additional circuitry, including for example, current control and/or voltage control, etc., other power consuming, power converting, power inverting and power generating elements, units, pieces, etc. including but not limited to those discussed herein. Switches or transistors 3742, 3744 provide shock hazard protection by effectively opening up the primary of the transformer 3740 thus not permitting current to flow, controlled through common gates 3746 in any suitable manner as disclosed above.

Turning to FIG. 38, the current (or voltage) control is accomplished by using switches represented by in the figure below as two transistors 3860, 3866 with common gates 3868 which each could consist of, for example, but not limited to, two or more back-to-back transistors including but not limited to MOSFETs connected such that the gates are connected together and sources are connected together, respectively, or BJTs with the bases connected together and emitters connected together, respectively or other types of compound, stacked, combined, etc., combinations of these switches, to shunt/short the excess current on the secondary or secondaries of the transformer 3840 before the excess current reaches (or voltage builds up at) either diode 3870 or 3872. Each of the ballast lamp outputs are respectively connected to capacitor pairs. For example, in an embodiment with four ballast lamp outputs as in a fluorescent lamp fixture configured to hold four fluorescent tubes, as shown in FIG. 38, one ballast lamp output is connected across capacitors 3800 and 3820, a second ballast lamp output is connected across capacitors 3804 and 3824, a third ballast lamp output is connected across capacitors 3810 and 3830, and a fourth ballast lamp output is connected across capacitors 3814 and 3834. The input capacitors are in turn connected to, for example, one or more transformers 3840 (in many applications and implementations, only one transformer is needed/used; however more than one can and may be used, etc.; in some embodiments of the present invention, capacitors may be optional). Again, type of transformer may be used including ones that require full bridge rectifiers, synchronous rectifiers, silicon controlled rectifiers, etc. The center tap can be grounded 3874 or can be floating as a local ground node or reference node.

The rectified secondary output 3880, 3882, for example from diodes 3870, 3872, for example but not limited to, is then fed to the lighting or additional circuitry, including for example, current control and/or voltage control, etc. Switches or transistors 3842, 3844 provide shock hazard protection by effectively opening up the primary of the transformer 3840 thus not permitting current to flow, controlled through common gates 3846 in any suitable manner as disclosed above.

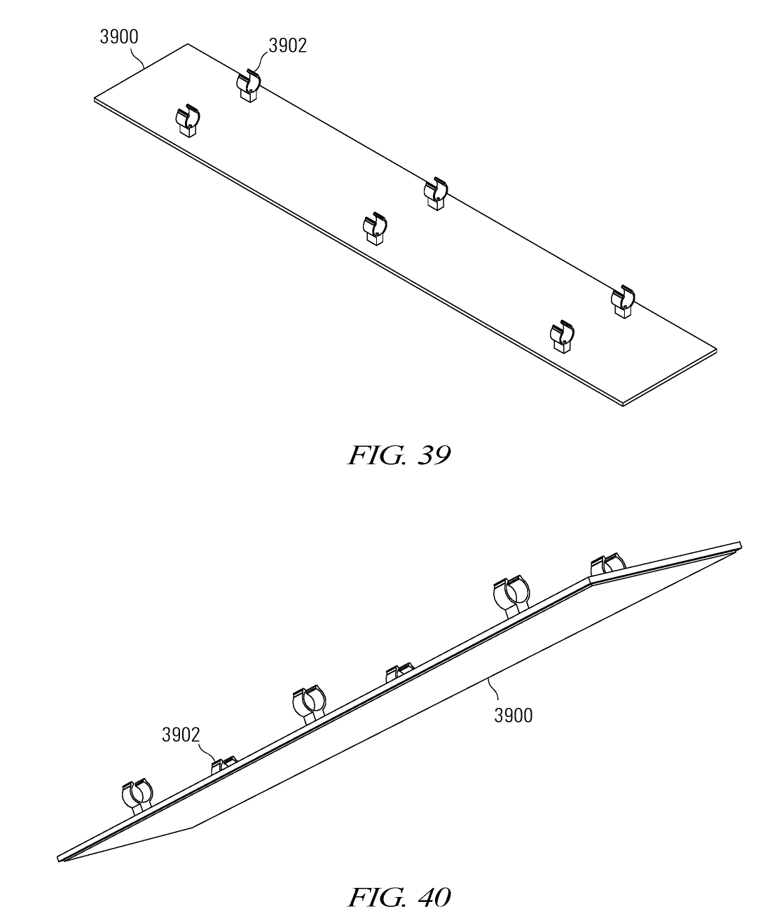

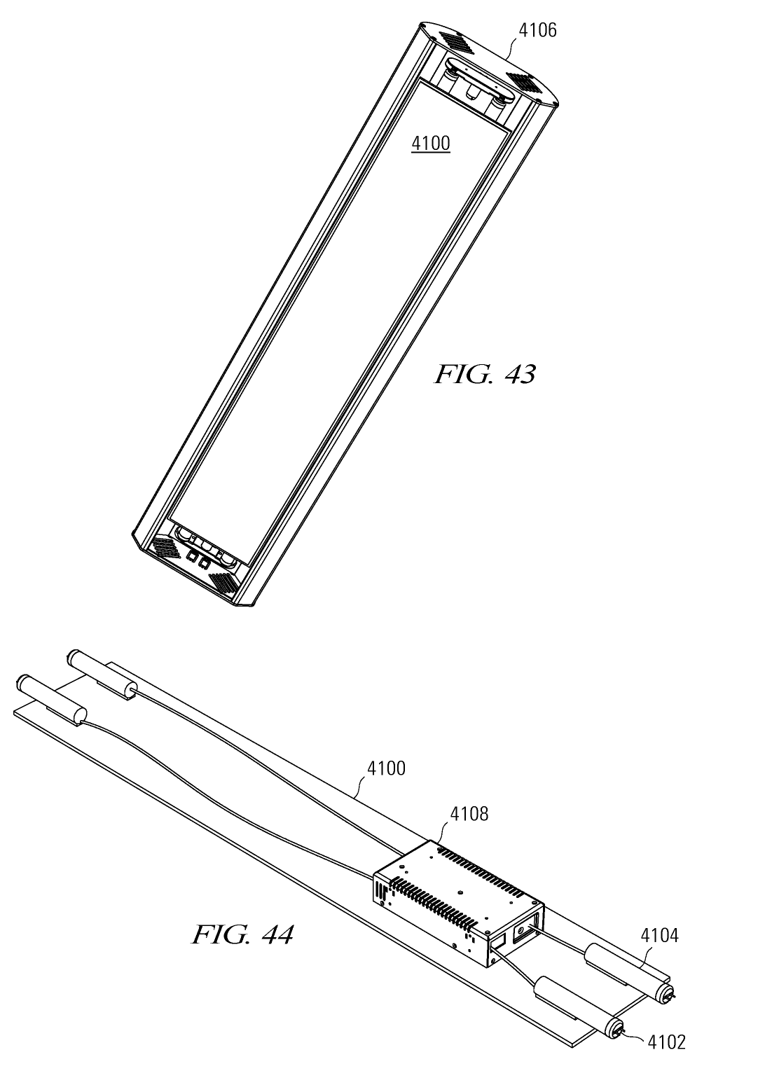

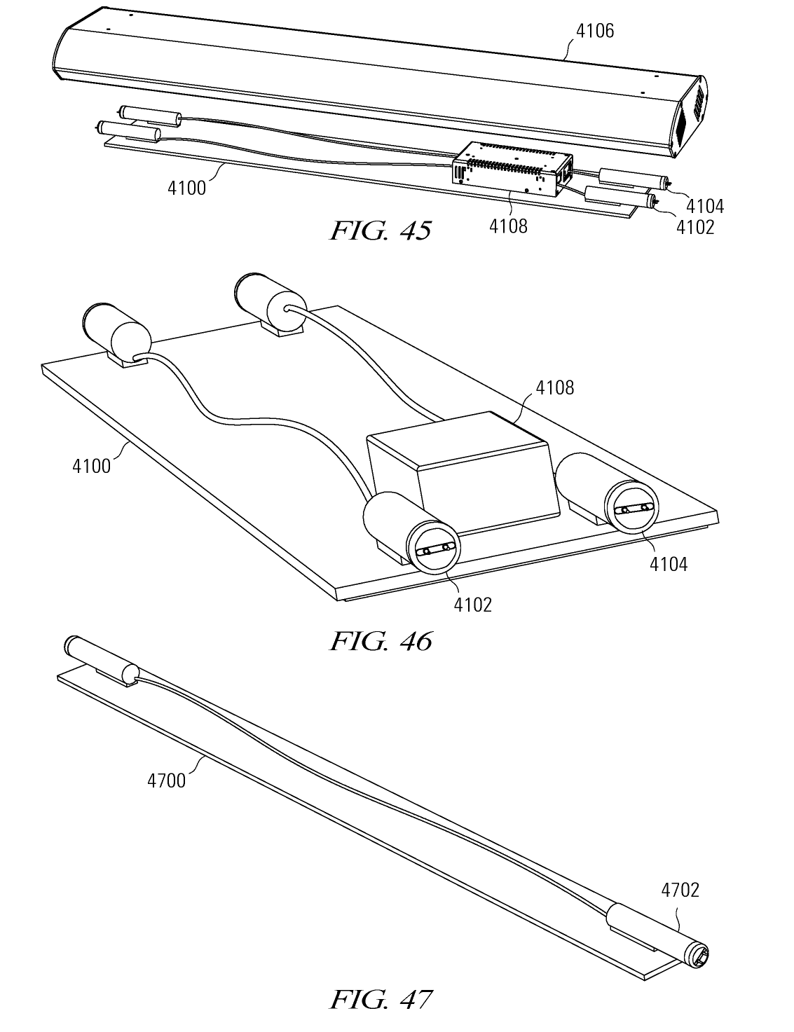

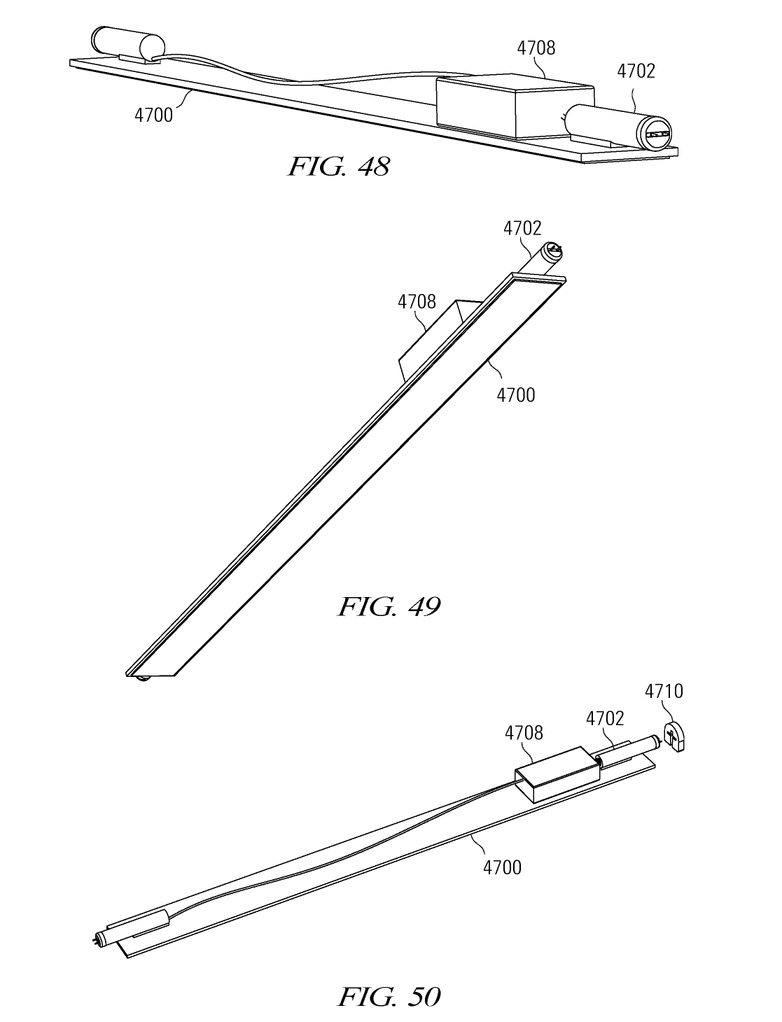

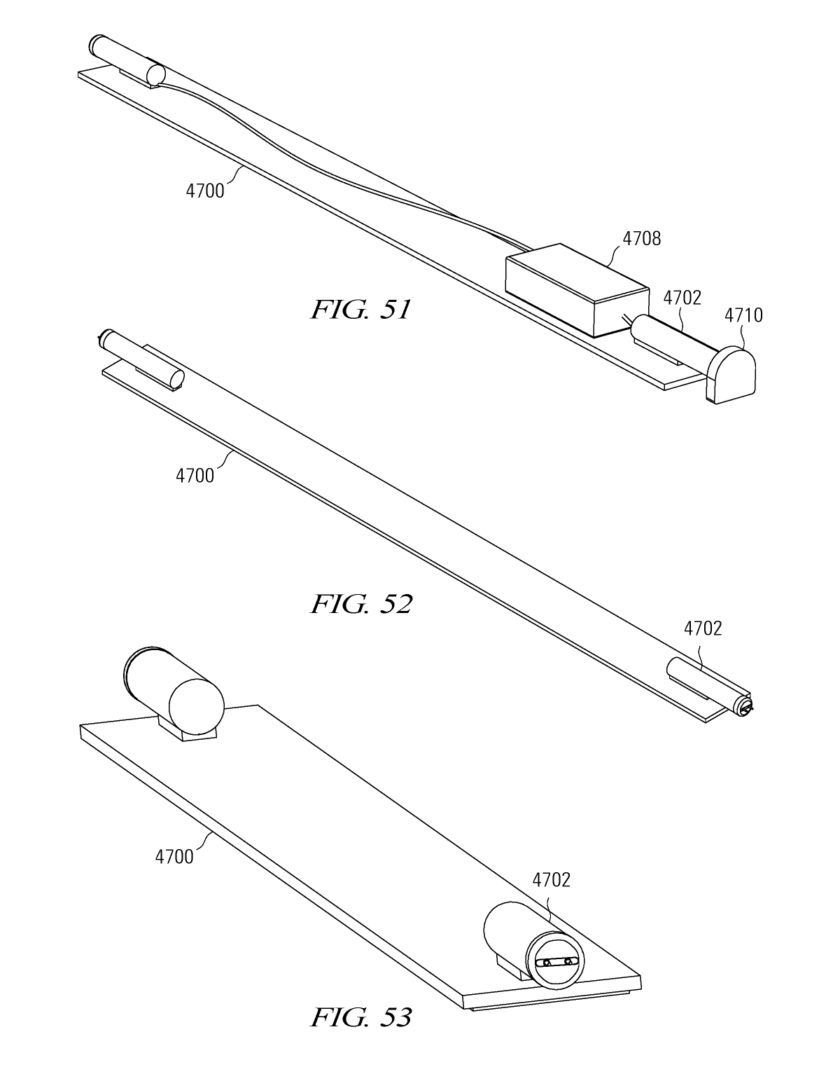

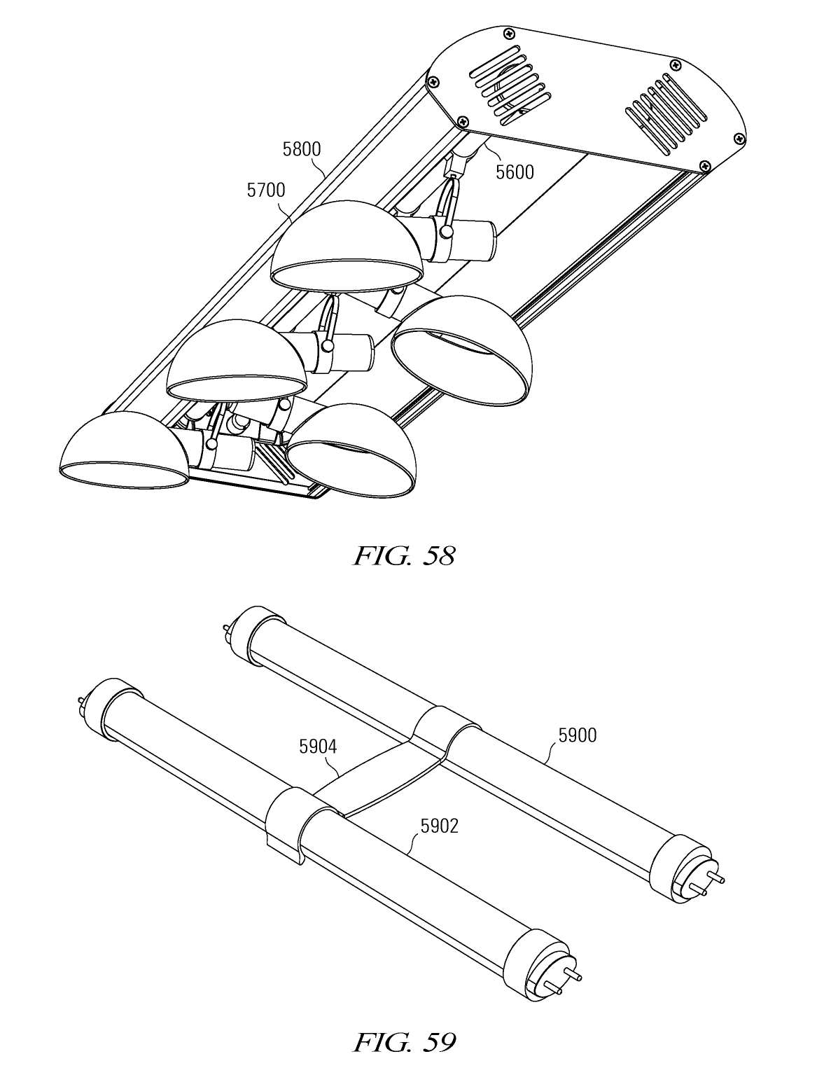

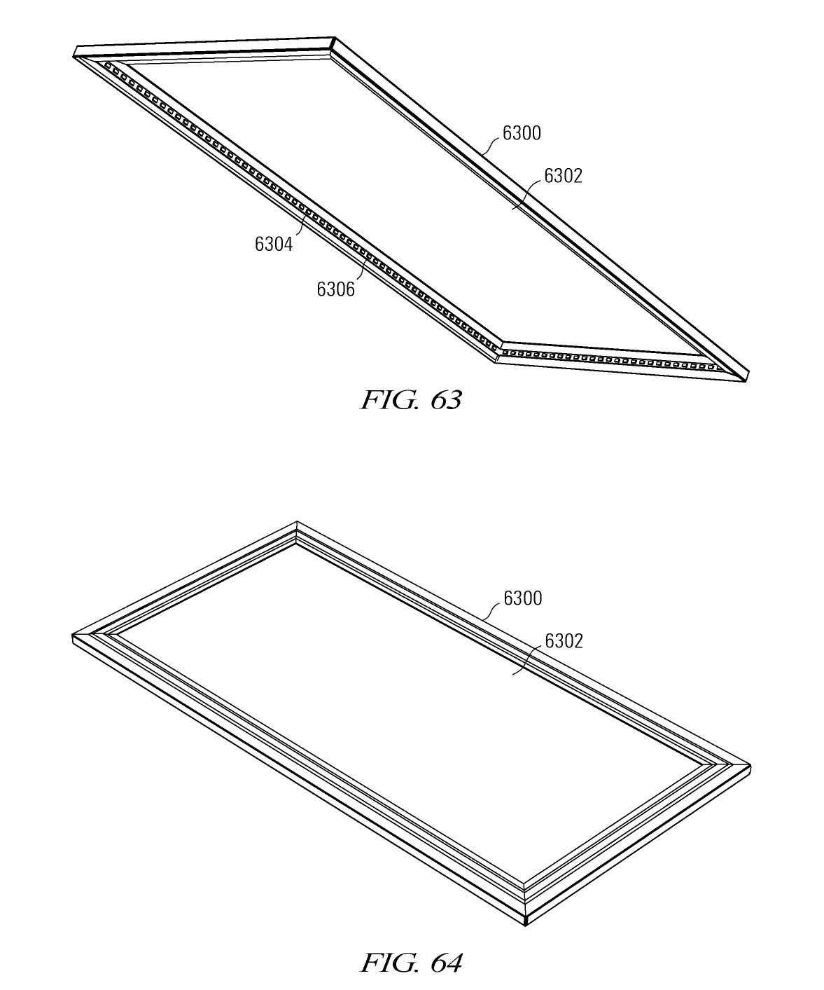

FIGS. 39-62 depict some example attachments and various configurations according to some embodiments of the present invention. The clamps/hooks depicted are used to support the lighting which can be bars of mostly any size and shape including but not limited to relatively long and thin ones such as nominally 1 ft.×4 ft. ½ ft. by 4 ft., 2 ft. by 4 ft., ½ ft.×3 ft., ½ ft.×3′h feet, etc. and which can consist of lighting of one or more colors including virtually any color such as but not limited to, white, red, blue, green, amber, cyan, orange, violet, yellow, etc., other colors, one or more color temperatures (CCTs) of white, combinations of these, etc. In some embodiments of the present invention, full spectrum ‘white’ light can be turned on to provide “Sun-like” illumination. In other embodiments of the present invention, the white light can be augmented with the color temperature set/controlled/modified by other colors including possible feedback with optical and/or spectral sensors/detectors/arrays/etc. In yet other embodiments of the present invention full spectrum Sun emulation can be accomplished by the proper selection of color light sources such as LEDs, OLEDs, quantum dots (QDs), PC-SSLs, etc., combinations of these, etc. Embodiments of the present invention allow for protection against too much power to the various light source elements and colors to avoid, for example, degradation, damage and potential harm, etc. as well as short circuit protection of one or more of the SSLs or accessories.

FIGS. 39-40 depict a solid state fluorescent lamp replacement with a light emitting panel 3900 and clips (e.g., 3902) for attaching to a lamp replacement bar mountable in a fluorescent fixture in accordance with some embodiments of the invention. Such example light emitting panels or other light sources or fixtures can be affixed to, for example, luminaires and fixtures, by a number of ways including, for example but not limited to, using the bi-pin sockets (or quad pin socket or other types of fluorescent lamp, energy efficient lamp, compact fluorescent lamp, etc. sockets, adapters, tombstones, etc.) for both power and mechanical support as depicted in the figures below where a plastic, metal, glass, other materials, combinations of other materials may be used to have a circular, square, rectangular, etc. shaped rod, bar, pole, etc. to act as both a conduit of electricity from the ballast and a support and/or enclosure for the power supply and lighting of the present invention. Power rails for example at 180 degrees from each other can be included in embodiments and implementations of the present invention to provide power to the present invention in a number of ways including clipping on light sources of bar, flat, tile, panel, PAR, track, down light, accent, string, round, square, rectangular, irregular shape(s), other types, including those discussed previously, etc., all and any type of existing light, light/lamp type, lighting source, lighting fixtures, form factor, size, shape, etc., combinations of these and others, etc. In addition, the fixture/luminaire can also be used for support as well as surrounding fixtures, supports, ceiling, wall, floor, grids, etc. The present invention allows for turnable/twistable connections to, for example, the bi-pins of the fluorescent light sockets/fixtures that may also contain switches for a number of functions and purposes including but not limited to safety, shock hazard, ballast phase, ballast polarity, power requirements, power usage and selection, etc. In some embodiments of the present invention these switches may be automated, remotely selected, remotely controlled and monitored, etc. In addition, other types of lighting as well as power consuming units appliances, cameras, including, but not limited to, security cameras, web cameras, personal cameras, DSLR cameras, phone cameras, CCD cameras, etc., IR cameras, night cameras, zooming cameras, combinations of these as well as battery chargers, cell phone chargers, tablet chargers, USB chargers, solar cell chargers, essentially chargers of any type and form, etc., other types of devices, things, internet of things (IOT), etc., including but not limited to those discussed herein may be used with the present invention.

The present invention applies to fluorescent lamps and fixtures and luminaires of all types and kinds—the present invention also applies in general to all types of high intensity discharge (HID) lighting including but not limited to mercury vapor lamps, metal-halide (MH) lamps, ceramic MH lamps, sodium-vapor lamps, xenon short-arc lamps, other types of arc lamps, sodium-based and other element-based lighting, gas discharge, etc., including but not limited to others discussed herein, etc.

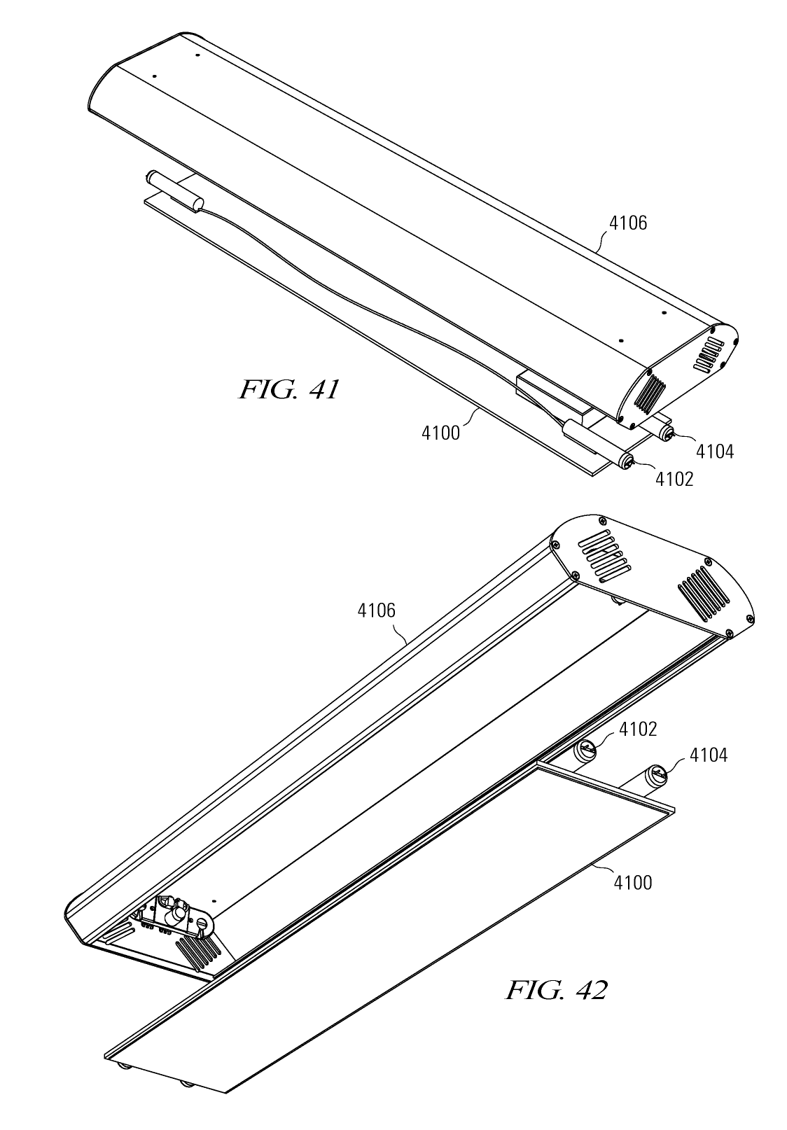

In some embodiments, as shown in FIGS. 41-46, a solid state light emitting panel 4100 is mounted and electrically connected to bi-pin connector mounts (e.g., 4102, 4104) which connect to tombstones in a fluorescent lamp fixture 4106. A power supply/supplies or converter 4108 can be included to convert/control etc. power for the solid state light emitting panel 4100 from the fluorescent lamp fixture 4106. The ballast provides power to the power supply/supplies or converter 4108 which, for example, takes as an input the high frequency AC from the ballast and converts to a DC constant current or constant voltage as output to for example power the light bars, panels, tiles, etc. (e.g., edge lit or direct lit or both, and/or other types, etc.) One or more electrical ballast connections can be provided on a panel 4100 and used with multiple lighting and other power consumers including but not limited to fans, heaters, coolers, other light types, televisions, battery chargers, for example, cell phones, tablets, portable devices of any type and form in general, etc. including but not limited to those discussed herein.

Appropriate connectors may be used on the external power supply versions such that the lamp electrical connections from the ballast outputs are securely and safely made so as to minimize, mitigate, eliminate, etc. the potential for human shock hazard to occur. In general there will be two or more connections/connectors from the ballast as an input to the present invention and two or more outputs from the present invention to the lighting of the present invention. As an example the two inputs from the output(s) of the ballast could consist of the aggregate of the N lamp output ballast that would be fed, for example, via capacitors to a single transformer or, as another example, it could consist of 2 times N outputs being fed to N transformers. In yet another example embodiment, it could consist of 2 times N outputs from the N lamp ballast being fed, for example, via capacitors to less than N transformers—for example, N/2 transformers. In any and all of these embodiments, male sockets can be used for the inputs from the ballasts as well as the female outputs from the external power supplies which can be designed and implemented to be safety connectors with the male side plugging into the female side such that no electrical contact is possible with the person or persons plugging the male input and output connectors into the female sockets. Any type of safety designed connector can be used with the present invention including but not limited to slotted connectors, twist connectors, spring loaded connectors, retractable connectors, etc., combinations of these, etc. Some embodiments of the present invention have the output connectors directly connected and incorporated into the lighting and other optional accessories such as charging ports, chargers, etc. and therefore either do not or may not require output connectors, etc. In still other embodiments the output is a relatively low, safe voltage (i.e., 12 V, 24 V, 30 V, 42 V, 48 V, etc.) and may not require as extensive safety measures to be used. In some embodiments of the present invention different sized input and output connectors can be used. In some embodiments of the present invention, AC voltages including but not limited to 120 VAC may be generated.

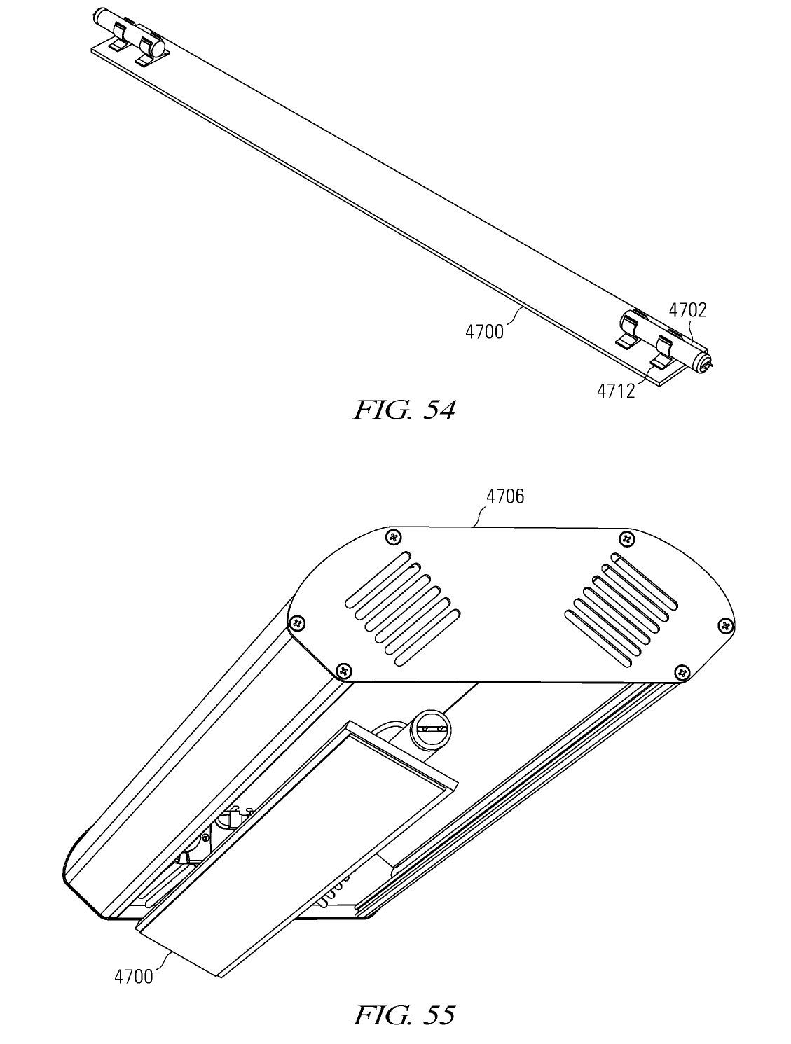

Turning to FIGS. 47-55, another solid state light emitting panel 4700 with bi-pin connector mounts (e.g., 4702) is adapted to mount to tombstones (e.g., 4710) in a fluorescent lamp fixture 4706 to replace one fluorescent lamp in accordance with some embodiments of the invention. In some embodiments, a power supply/supplies 4708 can be included to convert/control etc. power for the solid state light emitting panel 4700 from the fluorescent lamp fixture 4706. In some embodiments as in FIG. 52, bi-pin connector mounts (e.g., 4702) are mounted directly to the solid state light emitting panel 4700 or its substrate. In other embodiments as in FIG. 54, bi-pin connector mounts (e.g., 4702) are mounted to the solid state light emitting panel 4700 using clips (e.g., 4712). The lighting system is not limited to any method of attachments or to any type of mounting system for the lights. Light emitting panels can be mounted flat in fixtures or can be mounted at angles as shown in FIG. 55, and in some embodiments, can be turned, tilted, pivoted, etc., either manually or by motorized control.

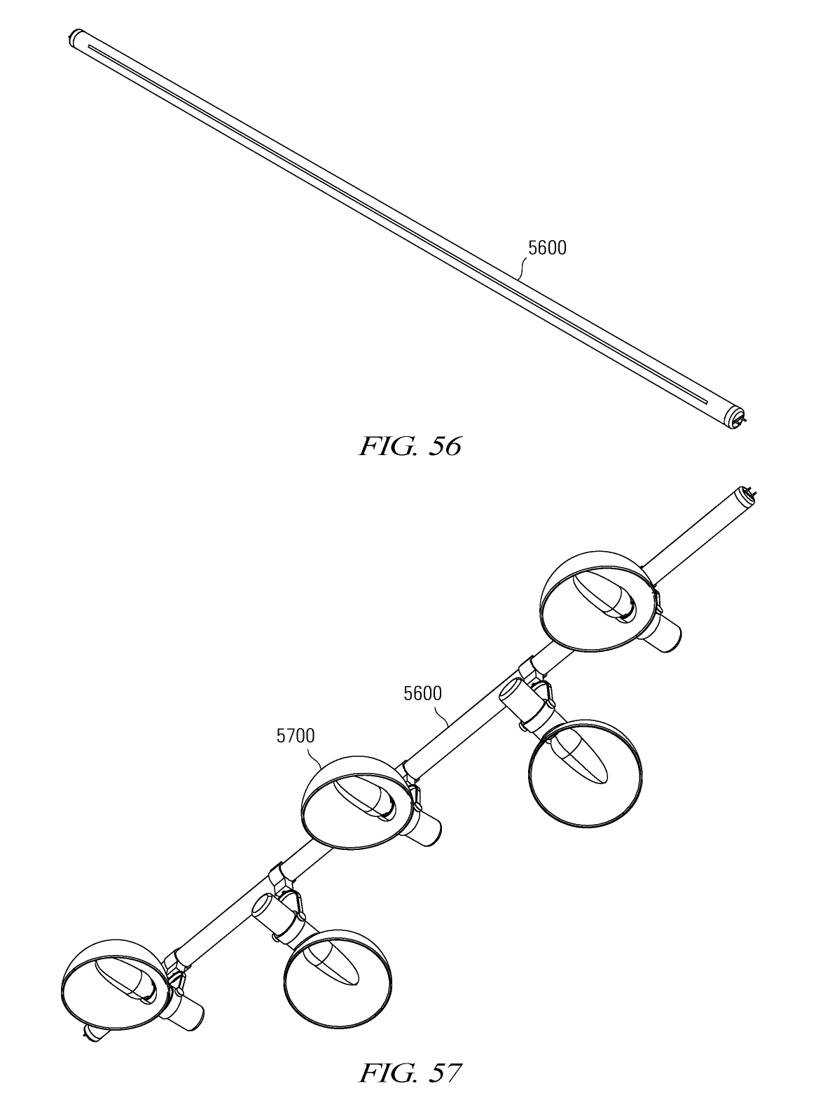

Turning to FIG. 56, a fluorescent replacement power buss 5600 is depicted in accordance with some embodiments of the invention. Again, the fluorescent replacement power buss 5600 can be fabricated using plastic, metal, glass, other materials, combinations of other materials with appropriate electrical conductors to have a circular, square, rectangular, etc. shaped rod, bar, pole, etc. to act as both a conduit of electricity from the ballast and a support and/or enclosure for the power supply and lighting of the present invention. Power rails or strip connectors can be provided that are diametrically opposed to each other for example at 180 degrees from each other. Note that the power conversion/supply unit can be inside the tubes/bars/etc., external to the tubes/bars/etc., inside the lighting, or combinations of these as well as having some of the parts/components distributed among these, etc. The arrangement(s) depicted in the Figures is/are merely for example purposes and is not intended to be limiting in any way or form.

Any type of solid state light can be connected to the fluorescent replacement power buss 5600, such as, but not limited to, the solid state spot lights (e.g., 5700) depicted in FIG. 57 in accordance with some embodiments of the invention. The solid state spot lights or lamps (e.g., 5700) are attached to the power buss 5600 which provides power to the lamps (e.g., 5700) via power converted from the ballast. The power buss 5600 with the lamps (e.g., 5700) can be inserted and retrofitted into a fixture 5800 that contains one or more ballasts and previously contained fluorescent tube lamps.

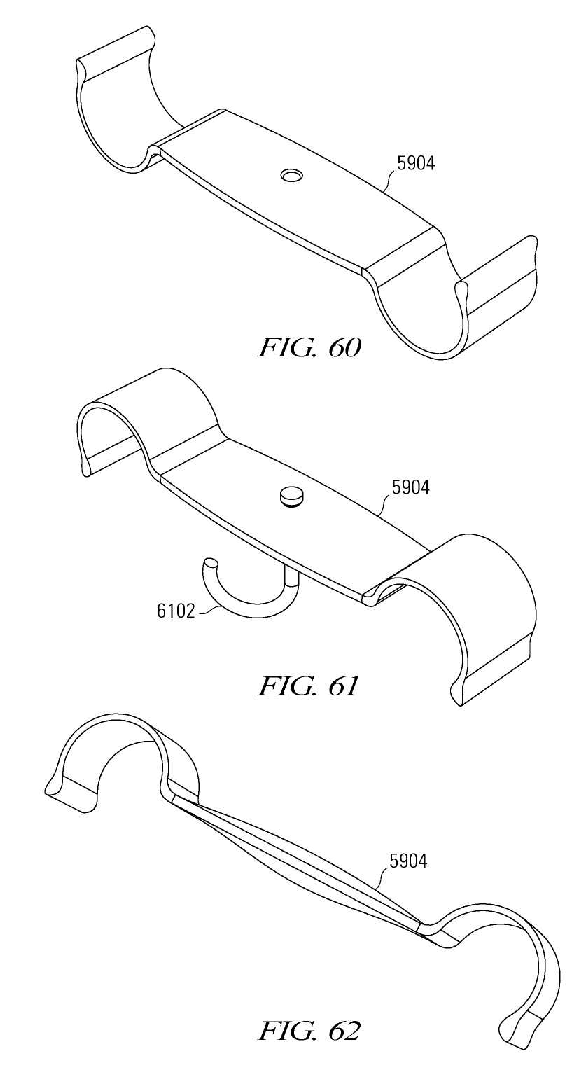

Turning to FIG. 59-62, multiple power busses 5900, 5902 can be combined or stabilized by a cross-bar 5904 which can also transmit electrical power, control signals, status and monitoring information, etc. between the power busses 5900, 5902. FIG. 59, multiple power busses 5900, 5902 can be combined or stabilized by a cross-bar 5904 which can also in some embodiments transmit electrical power, control signals, status and monitoring information, etc. between the power busses 5900, 5902. As shown in FIGS. 60-62, such a cross-bar 5904 can also be provided with one or more hooks 6102 which can be used to suspend and optionally connect lights or other devices. In some embodiments of the present invention, a low voltage AC or DC bus, for example, but not limited to 12 V, 24, V, etc. may be constructed from the embodiments and implementations of the present invention that either leave the ballast(s) intact and in use or remove the ballasts.

Turning now to FIGS. 63-64, an edge lit light bar/panel 6300, tile, etc. is depicted that can be of any color or can be of multiple colors including but not limited to white including multiple white color temperatures, red, green, blue, cyan, orange, yellow, amber, etc., combinations of these, etc. Such embodiments of the present invention can also include direct lit, back lit, etc., combinations of these, etc. and can consist of LEDs, OLEDs, quantum dots, etc. and can be remotely selected, controlled, monitored, log, etc. Thus, a light emitting panel 6302 can be combined with multiple point light sources (e.g., 6304, 6306), each of various and/or controllable colors.

The edge lit, direct lit, back lit, combinations of these, etc. can be powered by the ballast or ballasts (and/or AC line) and can be controlled, monitored, set, reset, etc. using wired, wireless, powerline, etc., combinations of these, etc. that have been discussed herein including but not limited to Bluetooth, WiFi, ISM, IEEE 801, DMX, DALI, RS 232, RS 485, SPC, SPI, U2C, USB, etc.

Embodiments of the present invention can also have lighting on the outside of, for example, the light bar, panel, tile, etc. including direct lit, edge lit, back lit, etc. Some example embodiments are shown below which can also include one or multiple LEDs, OLEDs, QDs including example configurations shown herein that can consist of one or more of white including more than one white color temperature, red, green, blue, amber, yellow, orange, etc. In addition, such lighting can be used to convey information about the status of a situation including flashing lights which may convey emergency situations, warning, greetings, alerts, alarms, attention, direction to move in/toward, location to go to, etc.

Some embodiments of the edge lit light bar/panel 6300 have light border that is lit red as an example; white as well as other colors such as blue, green, amber, orange, yellow, etc. can also be used and lit either individually or as combinations of, for example, discrete, blended or mixed, etc. colors or white color temperatures, etc. Note that the power conversion/supply unit can be inside the tubes/bars/etc., external to the tubes/bars/etc., inside the lighting, or combinations of these as well as having some of the parts/components distributed among these, etc. The arrangement(s) and colors depicted, shown and/or discussed in Figures is/are merely for example purposes and is not intended to be limiting in any way or form. Again, such embodiments can use/make/become/etc. a low voltage bus that can support a diverse variety of DC and/or AC powered devices including ones that require bucking down or boosting up using, for example, but not limited to, buck, boost, buck-boost, boost-buck, PWM, flyback, forward converters, Cuk, SEPIC, other switching and/or linear converters, inverters, etc., combinations of these, etc.

Some embodiments of the edge lit light bar/panel 6300 include a single or multiple (e.g., one or more) color edge lit system where the color green has been selected and powered. The edge lit light bar/panel 6300 can be back lit, edge lit, side lit, direct lit, etc. and can be white (including but not limited to one or more white color temperatures) or any color or combinations of colors or combinations of white(s) and colors, etc. including but not limited to white, red, blue, green, amber, orange, yellow, cyan, etc. In some embodiments of the present invention, blue and green may be used to provide health benefits including waking up to, light therapy for and to combat SAD, other diseases, ailments, illnesses, chronic conditions, etc. including blue light around 480 nm to stop melatonin whereas amber, red, certain shades, tones, etc. of orange, yellow, etc. may be used to promote melatonin and healthy sleep habits, behavior, circadian rhythm alignment, etc., combinations of these, etc.

In some embodiments and implementations of the present invention, one or more white(s) plus amber LEDs, OLED, and/or QDs are used; in other embodiments and implementations of the present invention, white(s) plus amber plus yellow LEDs, OLEDs, and/or QDs are used; in other embodiments and implementations of the present invention, white(s) plus blue plus amber plus yellow LEDs, OLEDs, and/or QDs are used; in other embodiments and implementations of the present invention, white(s) plus red plus green plus blue are used; in yet other embodiments and implementations of the present invention, one or more white plus red plus green plus blue plus amber, etc. are used. In such embodiments each color including each white color or color temperature as well as the other colors can in general be separately addressed and controlled, dimmed, monitored, logged, etc.

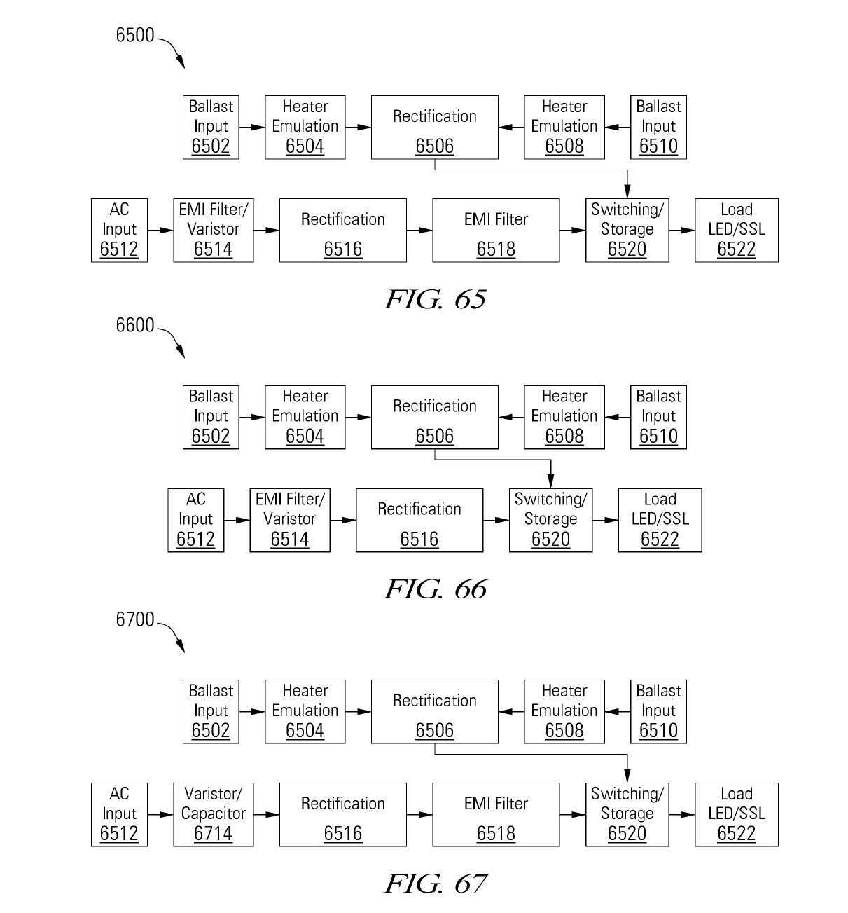

Turning to FIGS. 65-67, embodiments of a solid state fluorescent replacement lighting system are depicted in accordance with some embodiments of the invention. The block diagrams do not show optional elements such as a snubber, the feedback, set point, control, sense, other components, UVP, OVP, OTP, OCP, SCP, remote interfaces including but not limited to 0 to 10 V, 0 to 3V, microcontrollers, digital signal processors, Bluetooth controllers, radio chips, other digital and analog systems and accessories, etc., other wired, wireless and/or powerline communications, other control, monitoring, measuring, storage, memory, FLASH, EEPROM, etc., combinations of these, etc. In the embodiment of FIG. 65, a solid state fluorescent replacement lighting system 6500 derives power from ballast inputs 6502, 6510 through optional heater emulation circuits 6504, 6508 and rectifier 6506. Power can also or alternatively be derived from an AC input 6512 through rectifier 6516, with one or more optional EMI filters and varistor(s) 6514, 6518. Power is converted in switch/storage circuit 6520 to drive the solid state light(s) 6522.

The EMI components are for illustrative purposes only and are not limited in any way or form to what is shown and depicted herein and may contain, but are not limited to, inductors, chokes, beads, capacitors, resistors, other types of passive and active components, etc., combinations of these, etc.

In some embodiments of the present invention, the rectification can be shared and common to both the ballast and AC line powered modes of operation, etc. In some embodiments of the present invention, power can also be by DC voltage including lower voltage DC such as 12 volts DC or even ˜3 volts DC.

Turning to FIG. 66, a solid state fluorescent replacement lighting system 6600 derives power from ballast inputs 6502, 6510 through optional heater emulation circuits 6504, 6508 and rectifier 6506. Power can also or alternatively be derived from an AC input 6512 through rectifier 6516, with one or more optional EMI filters and varistor(s) 6514. Power is converted in switch/storage circuit 6520 to drive the solid state light(s) 6522.

Turning to FIG. 67, a solid state fluorescent replacement lighting system 6600 derives power from ballast inputs 6502, 6510 through optional heater emulation circuits 6504, 6508 and rectifier 6506. Power can also or alternatively be derived from an AC input 6512 through rectifier 6516, with one or more optional varistor(s)/capacitor 6714 and optional EMI filter 6518. Power is converted in switch/storage circuit 6520 to drive the solid state light(s) 6522.

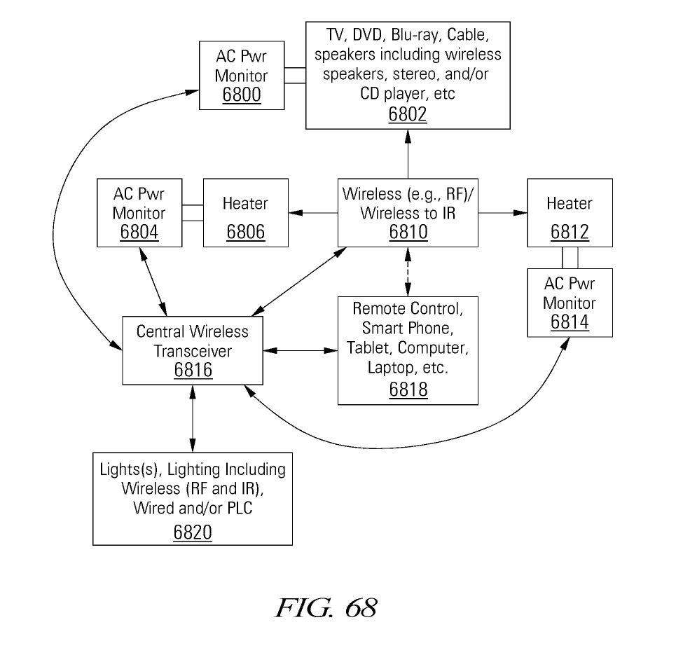

Turning now to FIG. 68, a block diagram depicts wireless/wired connections between components of a home automation system with mobile sensors incorporating a lighting system 6820 in accordance with some embodiments of the invention. One or more heaters (e.g., 6806, 6812), coolers or HVAC equipment can be controlled by wireless controllers or interpreters (e.g., 6810). Other elements (e.g., 6802) of the home automation system such as, but not limited to, lighting systems, entertainment systems or audiovisual equipment such as televisions, receivers, speakers, media players, audio players, communications systems, etc. can also be controlled by wireless controllers or interpreters (e.g., 6810). Power monitors 6810, 6804, 6814 can monitor and/or control the power provided to the heaters (e.g., 6806, 6812) and/or other elements (e.g., 6802) of the system, receiving control commands and/or sending status or other monitoring information to one or more central wireless transceivers 6816. One or more remote controls, smart phones, tablets, computers, laptops etc. (e.g., 6818) can also interface with the system, for example by wireless connection to central wireless transceivers 6816 and/or by optional wireless RF or IR links to interpreters (e.g., 6810). The lighting system 6820 can include wireless RF and/or IR links, and, in some embodiments, wired and/or PLC connections, and can be controlled by wireless controllers or interpreters (e.g., 6810) such as those disclosed in PCT/US15/12965 filed Jan. 26, 2015 for “Solid State Lighting Systems” which is incorporated herein by reference for all purposes.

An example of a wireless controlled SSL/LED FLR includes one or more of at least two different color temperature (e.g., cool and warm white) types such that with, for example, a diffuser the effective color can be varied from completely cool white to completely warm white with intermediate color blended combinations of cool and warm white in between. Note, other form factors, implementations, etc. including but not limited to having both cool and warm LEDs in the same wireless controlled FLR as well as novel form factors can be employed in implementations of the present invention. As also discussed herein, embodiments and implementations of the present invention can also include one or more SSLs/LEDs with different color temperatures as well as one or more colors or LEDs including but not limited to red, green, blue (RGB), red, green, blue, amber (RGBA), other colors, wavelengths, etc. of SSLs/LEDs, etc. An appropriately chosen capacitor value (which could consist of one or more capacitors) can be put across the two legs of the ballast through, for example, the tombstones that carry the current to drive the SSL (e.g., LED and/or OLED, QD) fluorescent lamp replacement to effectively reduce the maximum voltage that the ballast can put out.

Rapid start ballasts with heater connections may be made operable using resistors and/or capacitors. Certain implementations require less power and also evenly divide and resistance or reactive (e.g., capacitive and/or inductive) impedances so as to reduce or minimize power losses for the current supplied to the fluorescent lamp replacement(s). An example when having power supplied from an instant start or other ballast without heater(s) with only one electrical connection per ‘side’ of the fluorescent tube/lamp or fluorescent tube replacement (for a total of two connections) the resistors are effectively put into parallel thus reducing the resistance by a factor of four compared to being in serial for, for example, a heater emulation circuit or as part of a heater emulation circuit. Such heater circuits can contain resistors, capacitors, inductors, transformers, transistors, switches, diodes, silicon controlled rectifiers (SCR), triacs, other types of semiconductors and ICs including but not limited to op amps, comparators, timers, counters, microcontroller(s), microprocessors, DSPs, FPGAs, ASICs, CLDs, AND, NOR, Inverters and other types of Boolean logic digital components, combinations of the above, etc. In some embodiments of the present invention, a switch may be put (at an appropriate location) in between the ballast output and the fluorescent lamp/fluorescent lamp replacement such that there is no completion of current flow in the fluorescent lamp replacement to act as a protection including shock hazard protection for humans and other living creatures in the event of an improper installation or attempt at or during installation. The detection of a such a fault or improper installation can be done by any method including analog and/or digital circuits including, but not limited to, op amps, comparators, voltage reference, current references, current sensing, voltage sensing, mechanical sensing, etc., microcontrollers, microprocessors, FPGAs, CLDs, wireless transmission, wireless sensing, optical sensing, motion sensing, light/daylight/etc. sensing, gesturing, sonar, infrared, visible light sensing, etc. A microprocessor or other alternative including, but not limited to, those discussed herein may be used to enable or disable protection and may be combined with other functions, features, controls, monitoring, etc. to improve the safety and performance of the present invention including before, during, after dimming, etc. In embodiments of the present invention that include or involve buck, buck-boost, boost, boost-buck, etc. inductors, one or more tagalong inductors such as those disclosed in U.S. patent application Ser. No. 13/674,072, filed Nov. 11, 2012 by Sadwick et al. for a “Dimmable LED Driver with Multiple Power Sources”, which is incorporated herein for all purposes, may be used and incorporated into embodiments of the present invention. Such tagalong inductors can be used, among other things and for example, to provide power and increase and enhance the efficiency of certain embodiments of the present invention. In addition, other methods including charge pumps, floating diode pumps, level shifters, pulse and other transformers, bootstrapping including bootstrap diodes, capacitors and circuits, floating gate drives, carrier drives, etc. can also be used with the present invention. The present invention can work with programmable soft start ballasts including being able to also have a soft short at turn-on which then allows the input voltage to rise to its running and operational level can also be included in various implementations and embodiments of the present invention. Some embodiments of the present invention utilize high frequency diodes including high frequency diode bridges and current to voltage conversion to transform the ballast output into a suitable form so as to be able to work with existing AC line input PFC-LED circuits and drivers. Some other embodiments of the present invention utilize high-frequency diodes to transform the AC output of the electronic ballast (or the low frequency AC output of a magnetic ballast into a direct current (DC) format that can be used directly or with further current or voltage regulation to power and driver LEDs for a fluorescent lamp replacement. Embodiments of the present invention can be used to convert the low frequency (i.e., typically 50 or 60 Hz) magnetic ballast AC output to an appropriate current or voltage to drive and power LEDs using either or both shunt or series regulation. Some other embodiments of the present invention combine one or more of these. In some embodiments of the present invention, one or more switches can be used to clamp the output compliance current and/or voltage of the ballast.

Embodiments of the present invention include but are not limited to fluorescent and HID replacement lamps and lights of all types and all forms including but not limited to, for example, T5, T8 and T12 and PL13 to, for example PL42 lamps, compact fluorescents, energy saving fluorescent lamps, U-shaped fluorescent lamps, HID lamps of all types and kinds such that the SSL replacements including but not limited to LED, OLED, QD, micro LED, PC-SSL, etc., combinations of these, etc. can be dimmed/controlled by one or more of the following: AC line dimming (i.e., Triac, forward or reverse phase cut line dimming), wired analog or digital dimming (e.g., 0 to 10V, 0 to 3 V, other voltage range(s), PWM. DALI, DMX, RS485 and associated derivatives), SPI, I2C, serial, etc., others discussed herein, etc.), wireless (RF, IR, etc. including but not limited to, WiFi, Bluetooth, radios, including proprietary radios, other 2.4 GHz radios, ISM frequencies, etc.) as well as powerline communications. As an example embodiment, a T8 linear fluorescent lamp tube can be replaced with a SSL fluorescent lamp replacement (FLR) tube such that the T8 SSL FLR tube can be dimmed by wired and wireless methods, ways, techniques, etc. discussed herein when connected to a ballast including but not limited to an electronic ballast that is non-dimming and can be dimmed by wired and wireless methods, ways, techniques, etc. discussed herein as well as triac, forward and/or reverse phase dimming when connected to AC mains (i.e., typically 80 VAC to 480 VAC 50 or 60 Hz) or most magnetic ballasts as well as being powerline communications controllable and dimmable with the same universal AC input as well as DC from ˜3 VDC to over 500 VDC—all in the same T8 SSL FLR tube.

A light emitting diode (LED) system that can be used for, for example, photography and photographic applications to provide highly uniform lighting of a desired color temperature that can also be remotely dimmed, controlled, monitored, set, temporarily rapidly increased in intensity (flashed) etc. Such a lighting system can also include organic light emitting diodes (OLEDs), quantum dots (QDs). Such a lighting system can be dimmed, controlled, monitored, etc. wirelessly using, for example, WiFi, Bluetooth, industrial scientific and medical (ISM) frequencies, other protocols, technologies, techniques, interfaces, etc. The lighting system can be synchronized with other systems including but not limited to cameras including digital cameras via the wireless interface. The wireless communications, control, monitoring, etc. can be achieved with a dedicated device or devices, smart phones including iPhones, iPads, iPod touches, Android phones, tablets, etc., Windows smart phones, tablets, computers, laptops, desktops, other types of operating systems, computers, laptops, smart phones, tablets, etc. The lighting system can consist of edge lit, side lit, direct lit, back lit, etc. panels, tiles, portable lights, etc. made using, for example but not limited to, LEDs, OLED, QDs, fluorescent lighting, incandescent lighting, etc., combinations of these etc. The lighting can be, for example, white plus full spectrum natural lighting and can include any of the lighting discussed herein in any shape or form including without limitation the SSL discussed herein to among other things but not limited to create light boxes, screens, walls of light, specialized light enclosures, coordinated, special light effects including timed lighting effects, etc. with any combination of these and also with any combination of the present invention including but not limited to the lighting discussed herein. Light meters, color temperature meters, illumination meters, other types of meters and photographic equipment, instrumentation, metrics, meters, etc. can be used, interfaced, connected, synchronized, scheduled, etc. with the present invention in which for some embodiments and implementations can be controlled, monitored, sequenced, synchronized, activated, etc. from a smart phone, tablet, mini-tablet, laptop, computer, dedicated remote control, etc. In other embodiments various colors can be set including precisely color coordinate set. Embodiments of the present invention can be tiled together to create various two dimensional and three dimensional shapes with both white color and other color(s) control and maintenance. In addition to wireless, the present invention can also use wired interfaces, protocols, hardware, firmware, software, etc. including but not limited to, DMX, DALI, I2C, SPI, SPC, USB, other serial or parallel interfaces, etc. Embodiments and implementations of the present invention can also use both wireless including but not limited to WiFi, Bluetooth, Bluetooth low energy, Bluetooth or other similar radio with mesh capabilities, ZigBee, Zwave, IEEE 802, other wireless and wired approaches, methods, protocols, standards, interfaces, etc., combinations of these, etc. discussed herein and wired and combinations of these.

Embodiments of the present invention can provide both white plus full spectrum ‘natural’ light or a subset of the spectrum. In some embodiments and implementations of the present invention the white light can be white-tuned allowing the color temperature to be varied from low (i.e., 2000 or lower) Kelvin to high (i.e., 6500+) Kelvin and from warm to cool, etc. white temperature, etc. The present invention can work with all types of ballasts including magnetic and electronic ballasts and provide power for both lighting and all types of devices, electronics, circuits, devices, etc. including but not limited to cameras, microphones, speakers, bells, alarms, buzzers, security cameras, Web cameras, Internet cameras, infrared cameras, cameras with infrared lighting, infrared lighting in addition visible lighting, ultrasound, ultraviolet lighting, infrared, visible, and/or ultraviolet lasers including for security purposes with, for example photodetectors including modulated and detected laser/light and photodetector/sensor systems, combinations of these, etc., internet of things (IOT) devices, systems, components, etc., wireless transmitters, receivers, transceivers, repeaters, etc. for infrared, RF, millimeter wave, microwave, sub-millimeter wave, terahertz, gigahertz, WiFi, Bluetooth, ZigBee, Zwave, ISM, IrDA, etc. If the ballast should fail then, for example but not limited to, (1), the ballast can be replaced with a replacement ballast; (2), the ballast can be replaced with an AC to AC power supply that emulates the relevant behavior and performance of ballasts in general; (3) the external (or, in some cases, internal) power supply of the present invention can be bypassed and an AC to DC adapter can be used to provide power to, for example the lighting and potentially certain of the other elements, items, accessories, etc.; or (4) an universal AC voltage/ballast lamp output input embodiment/implementation of the present invention can be used such that when the ballast fails the present invention can be plugged into 50 or 60 Hz (or, for example 400 Hz) AC mains wall voltage. These examples are merely intended to be examples and should not be taken or construed as limiting in any way or form.

The example embodiments disclosed herein illustrate certain features of the present invention and not limiting in any way, form or function of present invention. The present invention is, likewise, not limited in materials choices including semiconductor materials such as, but not limited to, silicon (Si), silicon carbide (SiC), silicon on insulator (SOI), other silicon combination and alloys such as silicon germanium (SiGe), etc., diamond, graphene, gallium nitride (GaN) and GaN-based materials, gallium arsenide (GaAs) and GaAs-based materials, etc.

Remote dimming can be performed using a controller implementing motion detection, recognizing motion or proximity to a detector or sensor and setting a dimming level in response to the detected motion or proximity, or with audio detection, the power level of a wireless device, the absence or presence of a wireless device or one or more wireless devices, etc., for example detecting sounds or verbal commands to set the dimming level in response to detected sounds, volumes, or by interpreting the sounds, including voice recognition or, for example, by gesturing including hand or arm gesturing, etc. Some embodiments may be dual dimming, supporting the use of a 0-10 V dimming signal in addition to a Triac-based or other phase-cut or phase angle dimmer Some embodiments of the present invention may multiple dimming (i.e., accept dimming information, input(s), control from two or more sources). In addition, the resulting dimming, including current or voltage dimming, can be either PWM (digital) or analog dimming or both or selectable either manually, automatically, or by other methods and ways including software, remote control of any type including, but not limited to, wired, wireless, voice, voice recognition, gesturing including hand and/or arm gesturing, pattern and motion recognition, PLC, RS232, RS422, RS485, SPI, I2C, universal serial bus (USB), Firewire 1394, DALI, DMX, etc. Voice, voice recognition, gesturing, motion, motion recognition, etc. can also be transmitted via wireless, wired and/or powerline communications or other methods, etc. In some embodiments of the present invention speakers, earphones, headphones, microphones, etc. may be used with voice, voice recognition, sound, etc. and other methods, ways, approaches, algorithms, etc. discussed herein.

The present invention may use any type of circuit, integrated circuit (IC), microchip(s), microcontroller, microprocessor, digital signal processor (DSP), application specific IC (ASIC), field gate programmable array (FPGA), complex logic device (CLD), analog and/or digital circuit, system, component(s), filters, etc. including, but not limited to, any method to provide a switched signal such as a PWM drive signal to the switching devices. In addition, additional voltage and/or current detect circuits may be used in place of or to augment the control and feedback circuits.

Some embodiments of the present invention can accept the output of a fluorescent ballast replacement that is designed and intended for a LED or other type of SSL Fluorescent Lamp Replacement that is remote dimmable and can also be Triac, Triac-based, forward and reverse dimmer dimmable and incorporates all of the discussion above for the example embodiments. The remote fluorescent lamp replacement ballast can use or receive control signals/commands from, for example, but not limited to any or all of wired, wireless, optical, acoustic, voice, voice recognition, motion, light, sonar, gesturing, sound, mechanical, vibrational, and/or PLC, etc., combinations of these, etc. remote control, monitoring and dimming, motion detection/proximity detection/gesture detection, etc. In some embodiments, dimming or/other control can be performed using methods/techniques/approaches/algorithms/etc. that implement one or more of the following: motion detection, recognizing motion or proximity to a detector or sensor or a wireless signal or signal strength, etc. and setting a dimming level or control response/level including but not limited to turning on or off in response to the detected motion or proximity, or with audio detection, for example detecting sounds or verbal commands to set the dimming level in response to detected sounds, volumes, or by interpreting the sounds, including voice recognition or, for example, by gesturing including hand or arm gesturing, etc. sonar, light, mechanical, vibration, radar, ultrasonic, detection and sensing, etc. Some embodiments may be dual or multiple dimming and/or control, supporting the use of multiple sources, methods, algorithms, interfaces, sensors, detectors, protocols, etc. to control and/or monitor including data logging, data mining and analytics. Some embodiments of the present invention may be multiple dimming or control (i.e., accept dimming information, input(s), control from two or more sources).

Remote interfaces include, but are not limited to, 0 to 10 V, 0 to 2 V, 0 to 1 V, 0 to 3 V, etc., RS 232, RS485, DMX, WiFi, Bluetooth, ZigBee, IEEE 802, two wire, three wire, SPI, I2C, PLC, and others discussed in this document, etc. In various embodiments, the control signals can be received and used by the remote fluorescent lamp replacement ballast or by the LED, OLED and/or QD fluorescent lamp replacement or both. Such a Remote Controlled Florescent Ballast Replacement can also support color LED Fluorescent Lamp Replacements including single and multi-color including RGB, White plus red-green-blue (RGB) LEDs or OLEDs or other lighting sources, RGB plus one or more colors, red yellow blue (RYB), other variants, etc. Color-changing/tuning can include more than one color including RGB, WRGB, RGBW, WRGBA where A stands for amber, etc. 5 color, 6 color, N color, etc. Color-changing/tuning can include, but is not limited to, white color-tuning including the color temperature tuning/adjustments/settings/etc., color correction temperature (CCT), color rendering index (CRI), multiple color temperatures that can be mixed, dimmed, independently adjusted, set, selected, blended, etc. Color rendering, color monitoring, color feedback and control can be implemented using wired or wireless circuits, systems, interfaces, etc. that can be interactive using for example, but not limited to, smart phones, tablets, computers, laptops, servers, remote controls, personal digital assistants, etc. The present invention can use or, for example, make, create, produces, etc. any color of white including but not limited to soft, warm, bright, daylight, cool, etc. Color temperature monitoring, feedback, and adjustment can be performed in such embodiments of the present invention. The ability to change to different colors when using light sources capable of supporting such (i.e., LEDs, OLEDs and/or QDs including but not limited to red, green, blue, amber, one or more types, color temperatures, etc. of white LEDs or other SSLs and/or any other possible combination of LEDs, SSLs, and colors). Embodiments of the present invention has the ability to store color choices, selections, etc. and retrieve, restore, display, update, etc. these color choices and selections when using non-fluorescent light sources that can support color changing. Embodiments of the present invention also have the ability to change between various color choices, selections, and associated inputs to do as well as the ability to modulate the color choices and selections. A further feature and capability of embodiments of present invention is use of passive or active color filters and diffusers to produce enhanced lighting effects.

In addition, protection can be enabled (or disabled) by microcontroller(s), microprocessor(s), FPGAs, CLDs, PLDs, digital logic, etc. including remotely via wireless or wired connections, based on but not limited to, for example, a sequence of events and/or fault or no-fault conditions, sensor, monitoring, detection, safe operation, etc. An example of protection detection/sensing can include measuring/detecting/sensing lower current than expected due to, for example, a human person being in series with (e.g., in between) one leg of the LED, OLED and/or QD replacement fluorescent lamp and one side of the power being provided by the energized ballast. The present invention can use microcontroller(s), microprocessor(s), FPGA(s), other firmware and/or software means, digital state functions, etc. to accomplish protection, control, monitoring, operation, etc.

In addition to using a switching element, a linear regulation/regulator instead of switching regulation/regulator can be used or both linear and switching regulation and/or combinations of both can be used in embodiments of the present invention.

Rapid start ballasts with heater connections may be made operable using, for example but not limited to, resistors and/or capacitors as well as other types of components. Certain implementations may require less power and also evenly divide and resistance or reactive (e.g., capacitive and/or inductive) impedances so as to reduce or minimize power losses for the current supplied to the fluorescent lamp replacement(s). An example when having power supplied from an instant start or other ballast without heater(s) with only one electrical connection per ‘side’ of the fluorescent tube/lamp or fluorescent tube replacement (for a total of two connections) the resistors are effectively put into parallel thus reducing the resistance by, for example, a factor of two to four compared to being in serial for, for example, a heater emulation circuit or as part of a heater emulation circuit. Such heater circuits can contain resistors, capacitors, inductors, transformers, transistors, switches, diodes, silicon controlled rectifiers (SCR), triacs, other types of semiconductors and ICs including but not limited to op amps, comparators, timers, counters, microcontroller(s), microprocessors, DSPs, FPGAs, ASICs, CLDs, AND, NOR, Inverters and other types of Boolean logic digital components, combinations of the above, etc.

In some embodiments of the present invention, a switch may be put (at an appropriate location) in between the ballast output and the fluorescent lamp/fluorescent lamp replacement such that there is no completion of current flow in the fluorescent lamp replacement to act as a protection including shock hazard protection for humans and other living creatures in the event of an improper installation or attempt at or during installation. The detection of a such a fault or improper installation can be done by any method including analog and/or digital circuits including, but not limited to, op amps, comparators, voltage reference, current references, current sensing, voltage sensing, mechanical sensing, etc., microcontrollers, microprocessors, FPGAs, CLDs, wireless transmission, wireless sensing, optical sensing, motion sensing, light/daylight/etc. sensing, gesturing, sonar, infrared, visible light sensing, etc. A microprocessor or other alternative including, but not limited to, those discussed herein may be used to enable or disable protection and may be combined with other functions, features, controls, monitoring, etc. to improve the safety and performance of the present invention including before, during, after dimming, etc.

In embodiments of the present invention that include or involve buck, buck-boost, boost, boost-buck, etc. inductors, one or more tagalong inductors such as those disclosed in U.S. patent application Ser. No. 13/674,072, filed Nov. 11, 2012 by Sadwick et al. for a “Dimmable LED Driver with Multiple Power Sources”, which is incorporated herein for all purposes, may be used and incorporated into embodiments of the present invention. Such tagalong inductors can be used, among other things and for example, to provide power and increase and enhance the efficiency of certain embodiments of the present invention. In addition, other methods including charge pumps, floating diode pumps, level shifters, pulse and other transformers, bootstrapping including bootstrap diodes, capacitors and circuits, floating gate drives, carrier drives, etc. can also be used with the present invention.

The present invention can work with programmable soft start ballasts including being able to also have a soft short at turn-on which then allows the input voltage to rise to its running and operational level can also be included in various implementations and embodiments of the present invention.

Some embodiments of the present invention utilize high frequency diodes including high frequency diode bridges and current to voltage conversion to transform the ballast output into a suitable form so as to be able to work with existing AC line input PFC-LED circuits and drivers. Some other embodiments of the present invention utilize high-frequency diodes to transform the AC output of the electronic ballast (or the low frequency AC output of a magnetic ballast into a direct current (DC) format that can be used directly or with further current or voltage regulation to power and driver LEDs for a fluorescent lamp replacement. Embodiments of the present invention can be used to convert the low frequency (i.e., typically 50 or 60 Hz) magnetic ballast AC output to an appropriate current or voltage to drive and power LEDs using either or both shunt or series regulation. Some other embodiments of the present invention combine one or more of these. In some embodiments of the present invention, one or more switches can be used to clamp the output compliance current and/or voltage of the ballast. Various implementations of the present invention can involve voltage or current forward converters and/or inverters, square-wave, sine-wave, resonant-wave, etc. that include, but are not limited to, push pull, half-bridge, full-bridge, square wave, sine wave, fly-back, resonant, synchronous, etc.

For the present invention, in general, any type of transistor or vacuum tube or other similarly functioning device can be used including, but not limited to, MOSFETs, JFETs, GANFETs, depletion or enhancement FETs, N and/or P FETs, CMOS, PNP BJTs, triodes, etc. which can be made of any suitable material and configured to function and operate to provide the performance, for example, described above. In addition, other types of devices and components can be used including, but not limited to transformers, transformers of any suitable type and form, coils, level shifters, digital logic, analog circuits, analog and digital, mixed signals, microprocessors, microcontrollers, FPGAs, CLDs, PLDs, comparators, op amps, instrumentation amplifiers, and other analog and digital components, circuits, electronics, systems etc. For all of the example figures shown, the above analog and/or digital components, circuits, electronics, systems etc. are, in general, applicable and usable in and for the present invention.

Using digital and/or analog designs and/or microcontrollers and/or microprocessors any and all practical combinations of control, protection, sequencing, levels, etc., some examples of which are listed below for the present invention, can be realized.

In addition to these examples, a potentiometer or similar device such as a variable resistor may be used to control the dimming level. Such a potentiometer may be connected across a voltage such that the wiper of the potentiometer can swing from minimum voltage (i.e., full dimming) to maximum voltage (i.e., full light). Often the minimum voltage will be zero volts which may correspond to full off and, for the example embodiments shown here, the maximum will be equal to or approximately equal to the voltage on the negative input of, for example, a comparator.

Current sense methods including resistors, current transformers, current coils and windings, etc. can be used to measure and monitor the current of the present invention and provide both monitoring and protection.

In addition to dimming by adjusting, for example, a potentiometer, the present invention can also support all standards, ways, methods, approaches, techniques, etc. for interfacing, interacting with and supporting, for example, 0 to 10 V or 0 to 3 V or other range of dimming with a suitable reference voltage that can be remotely set or set via an analog or digital input such as illustrated in patent application 61/652,033 filed on May 25, 2012, for a “Dimmable LED Driver”, which is incorporated herein by reference for all purposes.

The present invention supports all standards and conventions for 0 to 10 V dimming or other dimming techniques. In addition the present invention can support, for example, overcurrent, overvoltage, short circuit, and over-temperature protection. The present invention can also measure and monitor electrical parameters including, but not limited to, input current, input voltage, power factor, apparent power, real power, inrush current, harmonic distortion, total harmonic distortion, power consumed, watthours (WH) or kilowatt hours (kWH), etc. of the load or loads connected to the present invention. In addition, in certain configurations and embodiments, some or all of the output electrical parameters may also be monitored and/or controlled directly for, for example, LED drivers and FL ballasts. Such output parameters can include, but are not limited to, output current, output voltage, output power, duty cycle, PWM, dimming level(s), provide data monitoring, data logging, analytics, analysis, etc. including, but not limited to, input and output current, voltage, power, phase angle, real power, light output (lumens, lux), dimming level if appropriate, kilowatt hours (kWH), efficiency, temperature including temperatures of components, driver, LED or OLED array or array or strings or other types of configurations and groupings, etc.

Embodiments of the present invention can also provide information on maintenance, malfunctions, errors, improper operation, too low of power, too high of power, efficiency performance, other issues, etc.

In place of the potentiometer, an encoder or decoder can be used. The use of such also permits digital signals to be used and allows digital signals to either or both locally or remotely control the dimming level and state. A potentiometer with an analog to digital converter (ADC) or converters (ADCs) could also be used in many of such implementations of the present invention.

The above examples and figures are merely meant to provide illustrations of the present and should not be construed as limiting in any way or form for the present invention.

In addition to the examples above and any combinations of the above examples, the present invention can have multiple dimming levels set by the dimmer in conjunction with the motion sensor and photosensor/photodetector and/or other control and monitoring inputs including, but not limited to, analog (e.g., 0 to 10 V, 0 to 3 V, etc.), digital (RS232, RS485, USB, DMX, SPI, SPC, UART, DALI, other serial interfaces, etc.), a combination of analog and digital, analog-to-digital converters and interfaces, digital-to-analog converters and interfaces, wired, wireless (i.e., RF, WiFi, ZigBee, Zwave, ISM bands, 2.4 GHz, Bluetooth, etc.), powerline (PLC) including X-10, Insteon, HomePlug, etc.), etc.

The present invention is highly configurable and words such as current, set, specified, etc. when referring to, for example, the dimming level or levels, may have similar meanings and intent or may refer to different conditions, situations, etc. For example, in a simple case, the current dimming level may refer to the dimming level set by, for example, a control voltage from a digital or analog source including, but not limited to digital signals, digital to analog converters (DACs), potentiometer(s), encoders, etc.

The present invention can have embodiments and implementations that include manual, automatic, monitored, controlled operations and combinations of these operations. The present invention can have switches, knobs, variable resistors, encoders, decoders, push buttons, scrolling displays, cursors, etc. The present invention can use analog and digital circuits, a combination of analog and digital circuits, microcontrollers and/or microprocessors including, for example, DSP versions, FPGAs, CLDs, ASICs, etc. and associated components including, but not limited to, static, dynamic and/or non-volatile memory, a combination and any combinations of analog and digital, microcontrollers, microprocessors, FPGAs, CLDs, etc. Items such as the motion sensor(s), photodetector(s)/photosensor(s), microcontrollers, microprocessors, controls, displays, knobs, etc. may be internally located and integrated/incorporated into the dimmer or externally located. The switches/switching elements can consist of any type of semiconductor and/or vacuum technology including but not limited to triacs, transistors, vacuum tubes, triodes, diodes or any type and configuration, pentodes, tetrodes, thyristors, silicon controlled rectifiers, diodes, etc. The transistors can be of any type(s) and any material(s)—examples of which are listed below and elsewhere in this document.

The dimming level(s) can be set by any method and combinations of methods including, but not limited to, motion, photodetection/light, sound, vibration, selector/push buttons, rotary switches, potentiometers, resistors, capacitive sensors, touch screens, wired, wireless, PLC interfaces, etc. In addition, both control and monitoring of some or all aspects of the dimming, motion sensing, light detection level, sound, etc. can be performed for and with the present invention.

Other embodiments can use other types of comparators and comparator configurations, other op amp configurations and circuits, including but not limited to error amplifiers, summing amplifiers, log amplifiers, integrating amplifiers, averaging amplifiers, differentiators and differentiating amplifiers, etc. and/or other digital and analog circuits, microcontrollers, microprocessors, complex logic devices (CLDs), field programmable gate arrays (FPGAs), etc.

The dimmer for dimmable drivers may use and be configured in continuous conduction mode (CCM), critical conduction mode (CRM), discontinuous conduction mode (DCM), resonant conduction modes, etc., with any type of circuit topology including but not limited to buck, boost, buck-boost, boost-buck, cuk, SEPIC, flyback, forward-converters, etc. The present invention works with both isolated and non-isolated designs including, but not limited to, buck, boost-buck, buck-boost, boost, cuk, SEPIC, flyback and forward-converters including but not limited to push-pull, single and double forward converters, current mode, voltage mode, current fed, voltage fed, etc. The present invention itself may also be non-isolated or isolated, for example using a tagalong inductor or transformer winding or other isolating techniques, including, but not limited to, transformers including signal, gate, isolation, etc. transformers, optoisolators, optocouplers, etc.

The present invention may include other implementations that contain various other control circuits including, but not limited to, linear, square, square-root, power-law, sine, cosine, other trigonometric functions, logarithmic, exponential, cubic, cube root, hyperbolic, etc. in addition to error, difference, summing, integrating, differentiators, etc. type of op amps. In addition, logic, including digital and Boolean logic such as AND, NOT (inverter), OR, Exclusive OR gates, etc., complex logic devices (CLDs), field programmable gate arrays (FPGAs), microcontrollers, microprocessors, application specific integrated circuits (ASICs), etc. can also be used either alone or in combinations including analog and digital combinations for the present invention. The present invention can be incorporated into an integrated circuit, be an integrated circuit, etc. It should be noted that the various blocks shown in the drawings and discussed herein may be implemented in integrated circuits along with other functionality. Such integrated circuits may include all of the functions of a given block, system or circuit, or a subset of the block, system or circuit. Further, elements of the blocks, systems or circuits may be implemented across multiple integrated circuits. Such integrated circuits may be any type of integrated circuit known in the art including, but are not limited to, a monolithic integrated circuit, a flip chip integrated circuit, a multichip module integrated circuit, and/or a mixed signal integrated circuit. It should also be noted that various functions of the blocks, systems or circuits discussed herein may be implemented in either software or firmware. In some such cases, the entire system, block or circuit may be implemented using its software or firmware equivalent. In other cases, the one part of a given system, block or circuit may be implemented in software or firmware, while other parts are implemented in hardware.

Embodiments of the present invention may also include short circuit protection (SCP) and other forms of protection including protection against damage due to other sources of power including but not limited to AC mains power lines and/or other types of devices, circuits, etc. Some embodiments of the present invention may use, for example, but are not limited to capacitors to limit the low frequency (examples include, but are not limited to, AC line mains at 50 Hz, 60 Hz, 400 Hz) voltage and/or current that can be applied to the load. In addition to capacitors, inductors and resistors may also be used in some embodiments of the present invention.

The present invention can also incorporate at an appropriate location or locations one or more thermistors (i.e., either of a negative temperature coefficient [NTC] or a positive temperature coefficient [PTC]) to provide temperature-based load current limiting.

Embodiments of the present invention can provide both white plus full spectrum ‘natural’ light or a subset of the spectrum. In some embodiments and implementations of the present invention the white light can be white-tuned allowing the color temperature to be varied from low (i.e., 2000 or lower) Kelvin to high (i.e., 6500 or higher) Kelvin and from warm to cool, etc. white temperature, etc. The present invention can work with all types of ballasts including magnetic and electronic ballasts and provide power for both lighting and all types of devices, electronics, circuits, devices, etc. including but not limited to cameras, microphones, speakers, bells, alarms, buzzers, security cameras, infrared cameras, cameras with infrared lighting, infrared lighting in addition visible lighting, ultrasound, ultraviolet lighting, infrared, visible, and/or ultraviolet lasers including for security purposes with, for example photodetectors including modulated and detected laser/light and photodetector/sensor systems, combinations of these, etc., internet of things (IOT) devices, systems, components, etc., wireless transmitters, receivers, transceivers, repeaters, etc. for infrared, RF, millimeter wave, microwave, sub-millimeter wave, terahertz, gigahertz, WiFi, Bluetooth, ZigBee, Zwave, ISM, IrDA, etc. If the ballast should fail then, for example but not limited to, (1), the ballast can be replaced with a replacement ballast; (2), the ballast can be replaced with an AC to AC power supply that emulates the relevant behavior and performance of ballasts in general; (3) the external (or, in some cases, internal) power supply of the present invention can be bypassed and an AC to DC adapter can be used to provide power to, for example the lighting and potentially certain of the other elements, items, accessories, etc.; or (4) an universal AC voltage/ballast lamp output input embodiment/implementation of the present invention can be used such that when the ballast fails the present invention can be plugged into 50 or 60 Hz (or, for example 400 Hz) AC mains wall voltage. These examples are merely intended to be examples and should not be taken or construed as limiting in any way or form.

As an example, when the temperature rises at the selected monitoring point(s), the phase dimming of the present invention can be designed and implemented to drop, for example, by a factor of, for example, two. The output power, no matter where the circuit was originally in the dimming cycle, will also drop/decrease by some factor. Values other than a factor of two (i.e., 50%) can also be used and are easily implemented in the present invention by, for example, changing components of the example circuits described here for the present invention. As an example, a resistor change would allow and result in a different phase/power decrease than a factor of two. The present invention can be made to have a rather instant more digital-like decrease in output power or a more gradual analog-like decrease, including, for example, a linear decrease in output phase or power once, for example, the temperature or other stimulus/signal(s) trigger/activate this thermal or other signal control.

In other embodiments, other temperature sensors may be used or connected to the circuit in other locations. The present invention also supports external dimming by, for example, an external analog and/or digital signal input. One or more of the embodiments discussed above may be used in practice either combined or separately including having and supporting both 0 to 10 V, 0 to 3 V, other analog dimming protocols, interfaces, approaches and digital dimming. The present invention can also have very high power factor. The present invention can also be used to support dimming of a number of circuits, drivers, etc. including in parallel configurations. For example, more than one driver can be put together, grouped together with the present invention. Groupings can be done such that, for example, half of the dimmers are forward dimmers and half of the dimmers are reverse dimmers. Again, the present invention allows easy selection between forward and reverse dimming that can be performed manually, automatically, dynamically, algorithmically, can employ smart and intelligent dimming decisions, artificial intelligence, remote control, remote dimming, etc.

The present invention may be used in conjunction with dimming to provide thermal control or other types of control to, for example, a dimming LED driver. For example, embodiments of the present invention or variations thereof may also be adapted to provide overvoltage or overcurrent protection, short circuit protection for, for example, a dimming LED or OLED driver, wall switch, separate wired, wireless, powerline control on/off, switch, etc., or to override and cut the phase and power to the dimming LED driver(s) based on any arbitrary external signal(s) and/or stimulus. The present invention can also be used for purposes and applications other than lighting—as an example, electrical heating where a heating element or elements are electrically controlled to, for example, maintain the temperature at a location at a certain value. The present invention can also include circuit breakers including solid state circuit breakers and other devices, circuits, systems, etc. that limit or trip in the event of an overload condition/situation. The present invention can also include, for example analog or digital controls including but not limited to wired (i.e., 0 to 10 V, RS 232, RS485, IEEE standards, SPI, I2C, other serial and parallel standards and interfaces, etc.), wireless including as discussed above, powerline, etc. and can be implemented in any part of the circuit for the present invention. The present invention can be used with a buck, a buck-boost, a boost-buck and/or a boost, flyback, or forward-converter design, topology, implementation, others discussed herein, etc.

A dimming voltage signal, VDIM, which represents a voltage from, for example but not limited to, a 0-10 V or 0 to 3 V or other range, etc Dimmer can be used with the present invention; when such a VDIM signal is connected, the output as a function time or phase angle (or phase cut) will correspond to the inputted VDIM.

Other embodiments can use comparators, other op amp configurations and circuits, including but not limited to error amplifiers, summing amplifiers, log amplifiers, integrating amplifiers, averaging amplifiers, differentiators and differentiating amplifiers, etc. and/or other digital and analog circuits, microcontrollers, microprocessors, complex logic devices, field programmable gate arrays, etc.

Some embodiments include a circuit that dynamically adjusts such that the output current to a load such as a LED and/or OLED array is essentially kept constant by, for example, in some embodiments of the present invention shorting or shunting current from the ballast as needed to maintain the output current to a load such as a LED array essentially constant. Some embodiments of the present invention may use time constants to as part of the circuit.

Some embodiments include a circuit to power a protection device/switch such that the switch is on unless commanded or controlled to be set off in the event/situation/condition of a fault hazard. Such a control can be implemented in various and diverse forms and types including, but not limited to, latching, hiccup mode, etc. In some embodiments of the present invention such a circuit may have a separate rectification stage. In and for various embodiments of the present invention, the device/switch may be of any type or form or function and includes but is not limited to, semiconductor switches, vacuum tube switches, mechanical switches, relays, etc.

Some embodiments include an over-voltage protection (OVP) circuit that shunts/shorts or limits the ballast output and/or the output to the load such as a LED array in the event that the output voltage exceeds a set value.

Some embodiments include an over temperature protection (OTP) circuit that shunts/shorts or limits the ballast output and/or the output to the load such as a LED array in the event that the temperature at one or more locations exceeds a set value or set values.

Embodiments of the present invention may also include short circuit protection (SCP) and other forms of protection including protection against damage due to other sources of power including but not limited to AC mains power lines and/or other types of devices, circuits, etc. Some embodiments of the present invention may use, for example, but are not limited to capacitors to limit the low frequency (examples include, but are not limited to, AC line mains at 50 Hz, 60 Hz, 400 Hz) voltage and/or current that can be applied to the load.

Embodiments of the present invention include, but are not limited to, having a rectification stage (such as, but not limited to) consisting of a single full wave rectification stage to provide power/current to the output load such as an LED output load and a rectification stage (such as, but not limited to) consisting of a single full wave rectification stage to provide power to, for example, the hazard protection circuit.

Remote dimming can be performed using a controller implementing motion detection, recognizing motion or proximity to a detector or sensor and setting a dimming level in response to the detected motion or proximity, or with audio detection, for example detecting sounds or verbal commands to set the dimming level in response to detected sounds, volumes, or by interpreting the sounds, including voice recognition or, for example, by gesturing including hand or arm gesturing, etc. Some embodiments may be dual dimming, supporting the use of a 0-10 V or 0 to 3 V, etc dimming signal in addition to a Triac-based or other phase-cut or phase angle dimmer. Some embodiments of the present invention may multiple dimming (i.e., accept dimming information, input(s), control from two or more sources). In addition, the resulting dimming, including current or voltage dimming, can be either PWM (digital) or analog dimming or both or selectable either manually, automatically, or by other methods and ways including software, remote control of any type including, but not limited to, wired, wireless, voice, voice recognition, gesturing including hand and/or arm gesturing, pattern and motion recognition, PLC, RS232, RS422, RS485, SPI, I2C, universal serial bus (USB), Firewire 1394, DALI, DMX, etc. Voice, voice recognition, gesturing, motion, motion recognition, signal strength including but not limited to wireless signal strength, etc. can also be transmitted via wireless, wired and/or powerline communications or other methods, etc. In some embodiments of the present invention speakers, earphones, microphones, etc. may be used with voice, voice recognition, sound, etc. and other methods, ways, approaches, algorithms, etc. discussed herein.

The present invention includes implementations that contain various other control circuits including, but not limited to, linear, square, square-root, power-law, sine, cosine, other trigonometric functions, logarithmic, exponential, cubic, cube root, hyperbolic, etc. in addition to error, difference, summing, integrating, differentiators, etc. type of op amps. In addition, logic, including digital and Boolean logic such as AND, NOT (inverter), OR, Exclusive OR gates, etc., complex logic devices (CLDs), field programmable gate arrays (FPGAs), microcontrollers, microprocessors, application specific integrated circuits (ASICs), etc. can also be used either alone or in combinations including analog and digital combinations for the present invention. The present invention can be incorporated into an integrated circuit, be an integrated circuit, etc.

The present invention, can include motion, noise, sound, and light/photodetection control and may also use other types of stimuli, input, detection, feedback, response, etc. including but not limited to sound, vibration, frequencies above and below the typical human hearing range, temperature, humidity, pressure, light including below the visible (i.e., infrared, IR) and above the visible (i.e., ultraviolet, UV), radio frequency signals, signal strength, sonar, radar, infrared in any form or way, ultrasonic, etc. combinations of these, etc.

For example, the motion sensor may be replaced or augmented with a sound sensor (including broad, narrow, notch, tuned, tank, etc. frequency response sound sensors) and the light sensor could consist of one or more of the following: visible, IR, UV, etc. sensors. In addition, the light sensor(s)/detector(s) can also be replaced or augmented by thermal detector(s)/sensor(s), etc.

The example embodiments disclosed herein illustrate certain features of the present invention and not limiting in any way, form or function of present invention. The present invention is, likewise, not limited in materials choices including semiconductor materials such as, but not limited to, silicon (Si), silicon carbide (SiC), silicon on insulator (SOI), other silicon combination and alloys such as silicon germanium (SiGe), etc., diamond, graphene, gallium nitride (GaN) and GaN-based materials, gallium arsenide (GaAs) and GaAs-based materials, etc. The present invention can include any type of switching elements including, but not limited to, field effect transistors (FETs) of any type such as metal oxide semiconductor field effect transistors (MOSFETs) including either p-channel or n-channel MOSFETs of any type, junction field effect transistors (JFETs) of any type, metal emitter semiconductor field effect transistors, etc. again, either p-channel or n-channel or both, bipolar junction transistors (BJTs) again, either NPN or PNP or both, heterojunction bipolar transistors (HBTs) of any type, high electron mobility transistors (HEMTs) of any type, unijunction transistors of any type, modulation doped field effect transistors (MODFETs) of any type, etc., again, in general, n-channel or p-channel or both, vacuum tubes including diodes, triodes, tetrodes, pentodes, etc. and any other type of switch, etc.

For the present invention, in general, any type of transistor or vacuum tube or other similarly functioning device can be used including, but not limited to, MOSFETs, JFETs, GANFETs, depletion or enhancement FETs, N and/or P FETs, CMOS, PNP BJTs, triodes, etc. which can be made of any suitable material and configured to function and operate to provide the performance, for example, described above. In addition, other types of devices and components can be used including, but not limited to transformers, transformers of any suitable type and form, coils, level shifters, digital logic, analog circuits, analog and digital, mixed signals, microprocessors, microcontrollers, FPGAs, CLDs, PLDs, comparators, op amps, instrumentation amplifiers, and other analog and digital components, circuits, electronics, systems etc. For all of the example figures shown, the above analog and/or digital components, circuits, electronics, systems etc. are, in general, applicable and usable in and for the present invention.

Implementations of the present invention are designed to be a cost-effective and complete solution that provides both forward and backward compatibility which is also ideal for retrofits and can use either wireless or wire (or both) communications.

Implementations of the present invention include comprehensive sensing and monitoring. Implementations of the present invention can be Web-based and/or WiFi-based (or other) and interface with smart phones, tablets, other mobile devices, laptops, computers, dedicated remote units, etc. and can support a number of wireless communications including, but not limited to, IEEE 802, ZigBee, Bluetooth, ISM, WiFi, proprietary radio, other radio frequencies, other frequencies in the electromagnetic spectrum, other protocols, standards, interfaces, etc.

Implementations of the present invention can include, but not limited to, dimmers, drivers, power supplies of all types, switches, motion sensors, light sensors, temperature sensors, daylight harvesting, other sensors, thermostats and more and can include monitoring, logging, analytics, etc.

Embodiments of the present invention support and can include color changing, color tuning, etc. lights with numerous ways to interact with the lights.

Embodiments of the present invention can be integrated with video, burglar, fire alarm, etc. components, systems.

Other features and functions include but are not limited to detecting the frequency using a microprocessor, microcontroller, FPGA, DSP, etc. Use a switch including, for example, a transistor such as a field effect transistor (FET) such as a MOSFET or JFET to, for example, either turn on or turn off a circuit that operates in either ballast mode or AC line mode depending on the amplitude of the signal or with the inclusion of a time constant, the average, RMS, etc. voltage level. Embodiments of the present invention removes the requirement that a reference level and a comparison to the reference level is required to detect the amplitude of the waveform

The present invention can also have sirens, microphones, speakers, earphones, headphones, emergency lights, flashing lights, fans, heaters, sensors including, but not limited to, temperature sensors, humidity sensors, moisture sensors, noise sensors, light sensors, spectra sensors, infrared sensors, ultraviolet sensors, speech sensors, voice sensors, motion sensors, acoustic sensors, ultrasound sensors, RF sensors, proximity sensors, sonar sensors, radar sensors, etc., combinations of these, etc.

The present invention can also provide two or more side (multi-side) lighting for example, for a FLR where one side contains SSL that, for example, consists of white color or white colors of one or more color temperatures and another side contains SSL or other lighting of one or more wavelengths such as red, green, blue, amber, white, yellow, etc., combinations of these, subsets of these, etc. The two or more sided lighting can perform different functions—for example, the side that is primarily white or all white light of one or more color temperatures can provide primary lighting whereas the side that has one or more color/wavelengths of light can provide indication of location, status, code level in, for example, a hospital (i.e., code red, code blue, code yellow, etc.), accent lighting, mood lighting, location indication, emergency information and direction, full spectrum lighting, etc.

The present invention can work with all types of communications devices including portable communications devices worn by individuals, walkie-talkie types of devices, etc.

The present device can use combinations of wireless and wired interfaces to control and monitor; for example for a linear or other fluorescent replacement for, for example, but not limited to, T4, T5, T8, T9, T10, T12, PL, PLC, HID of any type, form, power level, etc., other lamp types, etc. discussed herein, etc., one (or more) of the replacement lamps can be wireless with wired connections from the one (or more) replacement lamp(s) to the other replacement lamps such that the one or more wireless replacement lamps acts as a master receiving and/or transmitting information, data, commands, etc. wirelessly and passing along or receiving information, data, commands, etc. from the other remaining wired slaved units. In other embodiments one or more wired masters/leaders may transfer, transmit, or receive, etc. information, data, commands from other wireless and/or wired equipped fluorescent lamp replacements, etc. of combinations of these.

The present invention can also have one or more thermometers, thermostats, temperature controllers, temperature monitors, etc., combinations of these, etc. that can be wirelessly or wired interfaced controlled, monitored, etc. Such one or more thermometers, thermostats, temperature controllers, temperature monitors, etc., combinations of these, etc. can be connected/interfaced, for example, but not limited to, by Bluetooth, Bluetooth low energy, WiFi, IEEE 801, IEEE 802, ZigBee, Zwave, other 2.4 GHz and related/associated standards, protocols, interfaces, ISM, other frequencies including but not limited to, radio frequencies (RF), microwave frequencies, millimeter-wave frequencies, sub millimeter-wave frequencies, terahertz (THz), mobile cellular network connections, combinations of these. Wired connections, interfaces, protocols, etc. include but are not limited to, serial, parallel, UART, SPI, I2C, RS232, RS485, RS422, other RS standards and serial standards, interfaces, protocols, etc. powerline communications, interfaces, protocols, etc. including both ones that work on DC and/or AC, DMX, DALI, 0 to 10 Volt, other voltage ranges including but not limited to 0 to 3 Volt, 0 to 5 Volt, 1 to 8 Volt, etc.

In some embodiments of the present invention, the thermometer(s) and/or thermostats may be remotely located. In other embodiments of the present invention, such a temperature sensor or sensors or thermostat or thermostats can use wireless or wired units, interfaces. protocols, device, circuits, systems, etc. In some embodiments the thermometer(s) and/or thermostat(s) can communicate with each other and relay, share, augment, modify, interpret, add to, subtract from, and pass commands as well as provide information and data to one another.

In addition, embodiments of the present invention can use switches that are remotely controlled and monitored to detect the use of power or the absence of power usage, to open or close garage or other doors by locally and/or remotely sending signals to garage door openers including acting as a switch to complete detection circuits, remembering the status of garage door opening or closing, working with other motion sensors, photosensors, etc. horizontal/vertical detectors, inclinometers, etc., combinations of these, etc. Embodiments of the present invention can both control and monitor the status of the garage or other door and sound alarms, send alerts, flash lights including flashing white lights and/or one or more color/wavelength lights, turn on lights, turn off lights, activate cameras, record video, images, sounds, voices, respond to sounds, noise, movement, include and use microphones, speakers, earphones, headphones, cellular communications, etc., other communications, combinations of these, etc. Such embodiments and implementations can use Bluetooth, Bluetooth low energy, WiFi, IEEE 801, IEEE 802, ZigBee, Zwave, other 2.4 GHz and related/associated standards, protocols, interfaces, ISM, other frequencies including but not limited to, radio frequencies (RF), microwave frequencies, millimeter-wave frequencies, sub millimeter-wave frequencies, terahertz (THz), mobile cellular network connections, combinations of these. Wired connections, interfaces, protocols, etc. include but are not limited to, serial, parallel, SPI, I2C, RS232, RS485, RS422, other RS standards and serial standards, interfaces, protocols, etc. powerline communications, interfaces, protocols, etc. including both ones that work on DC and/or AC, DMX, DALI, 0 to 10 Volt, other voltage ranges including but not limited to 0 to 3 Volt, 0 to 5 Volt, 1 to 8 Volt, etc., relays, switches, transistors of any type and number, etc., combinations of these, etc.

The present invention also allows various types of radio frequency (RF) devices such as, but not limited to, window shades, drapes, diffusers, garage door openers, cable boxes, satellite boxes, etc. to be controlled and monitored by replacing and integrating these functions into implementations of the present invention including being able to synthesize and reproduce the RF signals which are typically in the range of less than 1 kHz to greater than 5 GHz using one or more RF synthesizers including ones based on phase lock loops and other such frequency tunable and adjustable circuits with may also employ frequency multiplication, amplification, modulation, etc., combinations of these, etc., amplitude modulation, phase modulation, pulses, pulse trains, combinations of these, etc.

A global positioning system (GPS) can be included in the present invention to track the location and, for example, to also make decisions as to where and when the present invention should do certain things including but not limited to turning on or off, dimming, turn on heat or cooling, control and monitor the lighting, etc., control, water, monitor the lawn and other plants, trees etc.

Embodiments of the present invention can use/incorporate/include/etc. thermal imagers including but not limited to IR imagers, IR imaging arrays, non-contact temperature measurements including point temperature and array temperature measurements including in lighting such as T8 replacements where the imagers are powered by, for example, but not limited to the ballast.

Embodiments of the present invention allow for dimming with both ballasts of any type including but not limited to electronic and magnetic ballasts and AC line voltage.

Implementations of the present invention can use, but are not limited to, Bluetooth, Bluetooth low energy, WiFi, IEEE 801, IEEE 802, ZigBee, Zwave, other 2.4 GHz and related/associated standards, protocols, interfaces, ISM, other frequencies including but not limited to, radio frequencies (RF), microwave frequencies, millimeter-wave frequencies, sub millimeter-wave frequencies, terahertz (THz), mobile cellular network connections, combinations of these. Wired connections, interfaces, protocols, etc. include but are not limited to, serial, parallel, SPI, I2C, RS232, RS485, RS422, other RS standards and serial standards, interfaces, protocols, etc. powerline communications, interfaces, protocols, etc. including both ones that work on DC and/or AC, DMX, DALI, 0 to 10 Volt, other voltage ranges including but not limited to 0 to 3 Volt, 0 to 5 Volt, 1 to 8 Volt, etc.

Embodiments of the present invention include SSL/LED Direct Fluorescent Tube Lamp Replacements that can be used, for example, but not limited to, for daylight harvesting/occupancy uses and applications.

Embodiments of the present invention uses wireless signals to both control (i.e., dim) the LED fluorescent lamp replacements (FLRs) and monitor the LED current, voltage and power. The present invention includes but is not limited to fluorescent lamp replacements that work directly with existing electronic ballasts and requires no re-wiring and can be installed in the same amount of time or less than changing a regular fluorescent lamp tube. These smart/intelligent LED FLRs are compatible with most daylight harvesting controls and protocols. Optional sensors allow for relative light output to be measured and wirelessly reported, monitored, and logged permitting analytics to be performed. Embodiments of the present invention come in a diversity of lengths including but are not limited to two foot and four foot T8 standard/nominal linear lengths as well as T12 as well as any other type of fluorescent and/or HID lamp including but not limited to those discussed herein. Additional optional input power measurements allow total power usage, power factor, input current, input voltage, input real and apparent power to also be measured thus allowing efficiency to be measured. The wireless signals can be radio signals in the industrial, scientific and medical (ISM) for lower cost and simplicity or ZigBee, ZWave, IEEE 802, or WiFi or Bluetooth or any type of form. In addition to occupancy/motion sensors, photo sensors and daylight harvesting controls, simple and low cost interfaces that allow existing other brands, makes, and models of daylight harvesting controls, photo sensors, occupancy/motion sensors to be connected to and control/dim embodiments of the wireless SSL/LED FLRs. The SSL FLR can be switched on and off millions of times without damage as well as be dimmed up and down without damage. The wireless communications can be encrypted and secure. Such embodiments of the present invention FLRs do not require or need a dimmable ballast and work with virtually any T8 electronic ballast from all major ballast manufacturers (optionally with most T12 electronic ballasts).

The present invention can have integrated motion sensor as part of the housing and can also use auxiliary motion sensors and can also have integrated light/photocell sensor as well as auxiliary. Such embodiments of the present invention can have the sensors discussed herein incorporated into the housing body or can have a cable or wireless connection to the sensors including having the one or more sensors mounted on the outside of the fixture, near the fixture or further away and more remote, etc. combinations of these, etc.

The present invention can also respond to proximity sensors including passive or active or both, as well as voice commands and can be used to turn on, turn off, dim, flash or change colors including doing so in response to an emergency situation. The present invention can use wireless, wired, powerline, combinations of these, etc., Bluetooth, RFID, WiFi, ZigBee, ZWave, IEEE 801, IEEE 802, ISM, any other type of sensor, detector, identifier, analog and/or digital ID, combinations of these including but not limited to those discussed herein, etc. In addition the present invention can be connected to fire alarms, fire alarm monitoring equipment, burglar and security protection company and services, health services, etc.

Embodiments of the present invention permits enhanced circadian rhythm alignment and maintenance using sources of light. Such sources of light include, but are not limited to, computer screens, monitors, panels, etc., tablet screens, smart phone screens, etc., televisions (TVs), LCD and CRT displays of any type or form, DVD and other entertainment lighting and displays containing LEDs, OLEDs, CCFLs, FLs, CRTs, etc., displays, monitors, TVs, OLED, LED, CCFL, FL, incandescent lighting, etc.

The present invention can use smart phones, tablets, computers, dedicated remote controls, to provide lighting appropriate for circadian rhythm alignment, correction, support, maintenance, etc. that can be, for example, coordinated wake-up and sleep times whether on a ‘natural’ or shifted (i.e., night workers, shift workers, etc.) to set and align their sleep patterns and circadian rhythm to appropriates phases including time shifts and time zone shifts due to work and other related matters.

The present invention can use external and internal information gathered from a number of sources including clocks, internal and external lighting, time of the year, individual, specific input, physiological signals, movements, monitoring of physiological signals, stimuli, including but not limited to, EEG, melatonin levels, urine, wearable device information, sleep information, temperature, body temperature, weather conditions, etc., combinations of these, etc.

The present invention can use TVs essentially of any type or form, including, but not limited to smart TVs, and related and similar items, products and technologies including, but not limited to, computer and other monitors and displays that can either be remotely or manually controlled and, in some embodiments, monitored. The present invention can use smart phones, tablets, PCs, remote controls including programmable remote controls, consoles, etc., combinations of these etc., to control and set the content of the lighting (e.g., white or blue-enriched, etc. combinations of these, etc. for wake-up; yellow, amber, orange, red, etc., combinations of these, etc. for sleep-time, etc.) automatically to assist in circadian rhythm, sleep, SAD mitigation, reduction, elimination, etc. In some embodiments of the present invention, music, sounds, white noise, sea shore sounds, sound effects, narratives, live audio, inspirational audio including previously recorded, generated, synthesized, etc., soothing sounds, familiar sounds and voices, etc. and combinations of these to go to sleep with. Jarring, buzzing, alarming, beeping, interrupting sounds, alarm clock sounds and noises, sleep disruptive sounds, noises and/or voices, etc. accompanied by white light, blue color/wavelength light including, but not limited to, slowing dimming up to a preset, optimum, and/or maximum brightness or setting, etc. for wake-up in the morning. Embodiments of the present invention can provide multiple wake-ups to the same location and/or different locations including other locations in homes, houses, hotels, hospitals, dormitories including school and military and other types of barracks, dormitories, etc., assisted living homes and facilities, chronic care facilities, rehabilitation facilities, etc., children's hospitals and care facilities, etc. group living, elder living, etc., children's rooms and other family members whether in the same physical location or in different physical locations, friends and family, clients, guests, travelers, jet lagged and sleep deprived people and personnel, etc.

The present invention can have integrated motion sensor(s) as part of the housing and can also use auxiliary motion sensors and can also have integrated light/photocell sensor as well as auxiliary. In some embodiments of the present invention, these can be stand-alone units that replace conventional fluorescent lamps including, but not limited to, T8, T12, T5, T10, T9, U-shaped, CFLs, etc. of any length, size and power as well as high intensity discharge lamps (HIDs) of any size, type, power, model, make, etc.

The present invention can also respond to proximity sensors including passive or active or both, as well as voice commands and can be used to turn on, turn off, dim, flash or change colors including doing so in response to an emergency situation. The present invention can use wireless, wired, powerline, combinations of these, etc., Bluetooth, RFID, WiFi, ZigBee, ZWave, IEEE 801, IEEE 802, ISM, etc. In addition the present invention can be connected to fire alarms, fire alarm monitoring equipment, home and/or business monitoring, protection services and companies, etc.

The present invention can use a BACNET to wireless converter box or BACNET to Bluetooth including Bluetooth low energy (BLE) converter. The present invention can also use infrared signals to control and dim the lighting and other systems as well as other types of devices including but not limited to heating and cooling, thermostats, on/off switches, other types of switches, etc.

The present invention can have the motion proximity sensor send signals back to the controller/monitor or other devices including but not limited to cell phones, smart phones, tablets, computers, laptops, servers, remote controls, etc. when motion or proximity is detected etc. Embodiments of the present invention can have on/off switches for the ballasts where the ballasts connect to the AC lines and/or also where the ballasts connect to the present invention, etc.

Embodiments and implementations of the present invention allow for optional add-ons including but not limited to field installable add-ons and/or upgrades including but not limited to hardware, firmware, software, etc., combinations of these, etc. including but not limited to wired, wireless or powerline control to be added later and interfaced to the present invention as well as allowing sensors such as daylight harvesting/photo/light/solar/etc. sensors as well as motion/PIR/proximity/other types of motion, distance, proximity, location, etc., sensors, detectors, technologies, etc., combinations of these, etc. to be used with the present invention.

The present invention provides a means to improve circadian rhythm by providing the appropriate wavelength and/or wavelengths of light at appropriate times.

Internal and external photosensors including wavelength specific or the ability to gather entire or partial spectrum, etc. and can use atomic clock(s) signals, other broadcast time signals, cellular phone, time, smart phone, tablet, computers, personal digital assistants, etc., remote control via dedicated units, smart phones, computers, laptops, tablets, etc.

The present invention can also have sirens, microphones, speakers, earphones, headphones, emergency lights, flashing lights, fans, heaters, sensors including, but not limited to, temperature sensors, humidity sensors, moisture sensors, noise sensors, light sensors, spectra sensors, infrared sensors, ultraviolet sensors, speech sensors, voice sensors, motion sensors, acoustic sensors, ultrasound sensors, RF sensors, proximity sensors, sonar sensors, radar sensors, cameras of any type and form including but not limited to one or more and more than one each of security cameras, infrared cameras, web cam (cameras), closed circuit cameras, etc., combinations of these, etc. The sound and/or noise sensors as well as other sensors, etc. can use one or more filters including one or more low pass, high pass, notch, bandpass including narrow bandpass filters, etc. Such filters can be realized by either or both analog and digital means, approaches, ways, functions, circuits, etc., combinations of these, etc. Such filter functions can be active or passive or both, can be manually and/or automatically set and adjustable, can be set, adjusted, programmed, etc. by an app, by other types and forms of software and hardware, by smart phone(s), tablet(s), laptops, servers, computers, other types of personal digital assistant(s), etc.

Embodiments of the present invention can have more than one wavelength or color of LEDs and/or SSLs and can include more than one array of LEDs, OLEDs, QDs, etc. that permit color selection, color blending, color tuning, color adjustment, etc. Embodiments of the present invention can include multiple arrays that can be switched on or off or in or out and/or dimmed with either power being supplied by a ballast or the AC line that can be remotely selected, controlled and monitored. Examples of the present invention include different wavelengths, combinations of colors and phosphors, etc. are used to obtain desired performance, effects, operation, use, etc. Embodiments can include one, two, three or more arrays of SSLs, including, but not limited to, side-by-side, 180 degrees from each other, on opposite sides, on multiple sides for example hexagon or octagon, etc. The SSLs including but not limited to LEDs, OLEDs, QDs, etc. may be put in series, parallel or combinations of series and parallel, parallel and series, etc. In other embodiments of the present invention, phosphors, quantum dots, and other types of light absorbing/changing materials that for example can effectively change wavelengths, colors, etc. for example by applying a voltage bias or electric field. The present invention can also take the form of linear fluorescent lamps from less than 1 foot to more than 8 feet in length and may typically be T4, T5, T8, T9, T10, T12, PL, PL-C, any and all other types of fluorescent and any and all types of HID lamps, etc., A-lamps, PAR 30, PAR 38, R20, R30, R40, BR30, other types of PAR, R, BR, halogen, low voltage, magnetic low voltage, transformer, etc., any known lamp, lamp type, lamp structure, including but not limited to those discussed, included, etc. herein, combinations of these, etc. Such embodiments of the present invention may use an insulating housing made from, for example but not limited to, glass or an appropriate type of plastic, which may or may not have a diffuser or be a diffuser in terms of the plastic. In some embodiments of the present invention plastic housings may be used that can include diffusers on the entire surface, diffusers on half the surface, diffusers on less than half the surface, diffusers on more than half of the surface, with the rest of the surface either being clear plastic, opaque plastic or a metal such as aluminum or an aluminum alloy.

Photon/wavelength conversion including down conversion can be used with the present invention including being able to adjust the photon/wavelength conversion electrically. Spectral/spectrum sensors can be used to detect the light spectral content and adjust the light spectrum by turning on or off certain wavelengths/colors of SSL. The spectral sensors could consist of color/wavelength sensitive detectors covering a range of colors/wavelengths of filters that only each only permit a certain, typically relatively narrow, range of wavelengths to be detected. As an example, red, orange, amber, yellow, green, blue, etc. color detectors could be included as part of the spectral/spectrum sensor or sensors. In some embodiments of the present invention, quantum dots can be used as part of and to implement the spectral/spectrum sensors, SSL including but not limited to LED, OLED, and/or QD lighting may use phosphor converted (PC) technologies, techniques, etc. and may be QC-based products, etc. In addition, microLEDs and related devices, technologies, techniques, approaches, etc. including PC-microLEDs may be used with and incorporated into embodiments and implementations of the present invention, etc.

Embodiments and implementations of the present invention can set user requirements, password priorities, permission levels, etc. for all or parts of the system including down to the individual lamp/bulb level which can/may be controlled, managed at a central or distributed level and can use mesh techniques to propagate information, commands, passwords, authentications, etc.

Implementations of the present invention can include and consist of any number and arrangement of smart dimmers (by wired, wireless, powerline communications, etc. combinations of these, etc.) including ones that connect directly to the AC power lines that can control, but are not limited to, one or more of, for example, but not limited to, as an example, FLRs, A-lamps, PAR 30, PAR 38, PLC lamps, R20, R30, MR16, track lighting, low voltage lighting including but not limited to legacy incandescent and halogen lighting as well as SSL/LED replacement lighting, dimmable compact florescent lamps, incandescent bulbs, halogen bulbs, etc. as well as smart dimmable (i.e., by wired, wireless, powerline communications, etc., combinations of these, etc.), infrared controlled devices including lighting of any type and form including dimmable and/or color-changing, color temperature (CCT) changeable/tunable lighting of any type and form, etc., heaters of any type or form, air conditioners of any type or form, color-changing, color-tunable, white color-changing, lighting of any type including but not limited to those discussed herein. Non-dimmable lamps and appliances and entertainment device can also be included in such implementations of the present invention and may be turned on and off by one or more of the smart on/off switches or a dimmer that is, for example, but not limited to, programmed to full on and full off only, etc. Such implementations of the present invention can also use one or more or all of the sensors, detectors, processes, approaches, etc. discussed herein and well as any other type or types of sensors, detectors, controls, etc. The smart lighting, dimmers, power supplies, sensors, controls, etc. can you any type or types of wired, wireless, and/or powerline communications. Any practical number of dimmers, lights, lighting, sensors, detectors, controls, monitoring, logging, analytics, heaters, air conditioners, fire, safety, burglar alarm(s), burglar protection, etc., appliances, entertainment devices, home safety, personal safety, thermometer(s), thermostat(s), humidifier(s), clock(s), including clock(s) of any type and form, timer(s), vents, registers, etc. for residential, home, and business HVAC, televisions, radios, stereos, printers, other office equipment and appliances, projectors including projectors for display video information, data, movies, word processing, presentations, including but not limited to power point presentations and PDF files, etc., other audio-visual equipment, accessories, components, including but not limited to screens, screens that can be lowered, raised, rolled up, etc. using electromechanical ways, methods, techniques, technologies, etc. including but not limited to motors, displays including computer monitors and smart TVs including ones with remote control capability such as an IR remote control, solar devices including but not limited to solar panels, inverters and converters for solar power generation, microgrids, minigrids, off-grid, grid power, back-up power, solar blankets, solar curtains, solar windows including but not limited to smart solar windows, solar drapes, solar blinds, etc. including but not limited to smart and intelligent solar systems, devices, components, etc.

The present invention provides for lighting that is highly configurable, controllable, customizable, sensor-rich, energy communication devices and includes, among other things, but not limited to, voice command, improved security and energy savings of up to 90% for starters.

Embodiments and implementations of the present invention can make buildings or all types, forms, uses, including but not limited to residential and commercial, smarter, more energy efficient with the sensors, SSL/LED lights, and controllers and other embodiments of the present invention that allow, for example, but are not limited to integrating the present invention into existing building energy management systems

Embodiments of the present invention can increase Lighting IQ, which allows the different kinds/types of smart, intelligent lighting to be incorporated including but not limited to: daylight harvesting to prevent needless use of over lighting of sunlit and other externally artificially lit rooms and extend bulb life coupled with simple, easy installation through, for example, but not limited to, plug-and-play, constant-lumens technology. In parking lots, the present invention will prevent needless over-lighting of these by using one or more of occupancy, ultrasonic, sonar, radar, noise, vision recognition, camera analysis, data mining, pattern recognition, etc., web cams, security cameras, inspection cameras, etc., motion sensors, etc. to ensure the parking lot or the path through the parking lot is well lit when and where it needs to be, and save energy by dimming or even turning off lights when they are not needed. Embodiments of the present invention will also help to create high IQ lighting environments with adaptive and color-changing, color tuning lights that help students from elementary through professional/graduate school learn, focus, stay attentive and awake or rest when and where needed. Other embodiments of the present invention include high IQ lighting for hospitals, laboratories and emergency applications and situations including but not limited to high quality health care, light therapy, light centric medical and health and healing applications, patient ability to adjust, control and be better with proper lighting, etc.

Embodiments of the present invention can improve security and performance while saving energy and money as well as the lighting having a dramatic positive effect in improving the appearance including but not limited to lights that can change color to suit mood, dim when no one is around and turn on when motion or noise is detected.

The present invention include but are not limited to intelligent lighting solutions related to the control, communication, analytics, sensing and monitoring technologies that can fundamentally change the power consumption and utility of lighting systems Embodiments of the present invention can use the lights to collect a wide variety of sensor information that can be used for, for example, but are not limited to, enhancing energy savings to improving security and efficiency.

Embodiments of the present invention allow for automatic and/or manual dimming coupled with monitoring ambient light and intelligently auto-dims in response. Dim level can also be adjusted manually or automatically including but not limited to timing, sequencing, synchronizing, etc.

Embodiments of the present invention allow for Plug-and-Play by for example but not limited to replacing fluorescent lamps (compact, PLC, and/or linear, etc.) with SSL/LED technology is as easy as plug-and-play—no re-wiring or ballast change required making your retrofit easy and cost effective with embodiments of the present invention that can also be directly powered by AC or DC. Embodiments of the present invention allow for the lighting to be accessed on the individual lamp level through, for example, but not limited to, Bluetooth and WiFi communication pathways

Implementations of the present invention allow for the SSL/LED power supply and driver to produce constant lumen SSL/LED output regardless and independent of type of ballast or lack of presence of ballast (i.e., can be wired directly to AC or DC power). Embodiments of the present invention allow for two way communication with the lighting using, for example, but not limited to, computer software, servers, tablets, smartphones, or local manual controls. Some embodiments of the present invention can include and/or work with cybersecure interfaces and protocol.

The operational lifetime of the SSL/LED lighting can be significantly extended with auto dimming Unlike incandescent or fluorescent lighting, the lifetime of LEDs is not shortened by frequent switching or thermal cycles.

Many implementations of the present invention can be configured to have autonomous control with each sensor or group of sensors interacting with the lighting autonomously, or other implementations of the present invention can be integrated into energy management systems to maximize energy savings and enhance the work environment, while providing detailed analytics and monitoring, including for marine and shipboard applications.

Embodiments of the present invention can be tuned to wavelengths that are important to the health of employees, patients or customers. Specific wavelengths can aid in Seasonal Affective Disorder (SAD) and help regulate circadian rhythms for better sleeping.

Embodiments of the present invention can be solar friendly and used with low-voltage DC, line-voltage AC or DC sockets, and ballasts without requiring power converters.

Embodiments of the present invention are backward (and forward) compatible and can be completely interoperable with existing energy management systems and can be used with different brands of equipment already installed.

While detailed descriptions of one or more embodiments of the invention have been given above, various alternatives, modifications, and equivalents will be apparent to those skilled in the art without varying from the spirit of the invention. Therefore, the above description should not be taken as limiting the scope of the invention, which is defined by the appended claims.

What is claimed is:

1. A lighting system comprising:
a plurality of light sources with multiple light colors configured to replace fluorescent lamps in a fluorescent lamp fixture; and
a power supply configured to power the plurality of light sources and to control an overall output color.

2. The lighting system of claim 1, wherein the plurality of light sources and the power supply are embodied in a fluorescent lamp replacement.

3. The lighting system of claim 2, wherein the power supply comprises an AC input and a ballast input configured to receive power from a fluorescent lamp ballast.

4. The lighting system of claim 3, wherein the power supply is configured to automatically select between providing power from the AC input or from the ballast input depending at least in part on whether the fluorescent lamp ballast is present in a fixture.

5. The lighting system of claim 3, wherein the power supply comprises a switch configured to select between providing power from the AC input or from the ballast input depending at least in part on whether the fluorescent lamp ballast is present in a fixture.

6. The lighting system of claim 3, wherein the power supply comprises a heater simulation circuit configured to simulate a fluorescent lamp heater circuit for the fluorescent lamp ballast.

7. The lighting system of claim 3, wherein the power supply comprises an overvoltage protection circuit connected to the ballast input.

8. The lighting system of claim 2, further comprising at least one sensor configured to monitor when a person is looking at the plurality of light sources.

9. The lighting system of claim 2, wherein the plurality of light sources comprises at least one blue OLED panel and at least one amber OLED panel.

10. The lighting system of claim 9, wherein the power supply is configured to control an output of the at least one blue OLED panel and the at least one amber OLED panels simultaneously to produce a white light output.

11. The lighting system of claim 1, further comprising a controller configured to control the overall output color based at least in part on time of day.

12. The lighting system of claim 1, further comprising a controller configured to turn on the plurality of light sources to wake a person as a light alarm.

13. The lighting system of claim 1, further comprising a microphone and a controller configured to control the plurality of light sources based at least in part on sound detected by the microphone.

14. The lighting system of claim 13, wherein the controller is configured to provide an indication of sound levels over a threshold by controlling the plurality of light sources.

15. The lighting system of claim 1, wherein the plurality of light sources comprise a combination of OLED and LED light sources.

16. The lighting system of claim 1, wherein the plurality of light sources comprise OLED panels placed in multi-sided arrangement.

17. The lighting system of claim 16, wherein each side of the multi-sided OLED panels is configured to output light of a different color selected to affect circadian rhythms differently.

18. The lighting system of claim 2, wherein the power supply is configured to receive power from multiple ballast outputs.

19. The lighting system of claim 18, wherein different power inputs to the power supply are isolated.

20. The lighting system of claim 18, wherein different power inputs to the power supply are non-isolated.
